SEQUENCE LISTING (205 sequences)

SEQ ID NO: 1 - RNA, 123 nt, synthetic construct
  misc_feature 1..123: note = SEQ ID NO: 1;
  source 1..123: mol_type = other RNA
ccaggtgtctttcaaagtaggttttagagctagaaatagcaagttaaaataaggctagtccgttatcaacttgaaaaagtggcaccgagtcggtgcagcctccgacttctttgaaagacacct

SEQ ID NO: 2 - RNA, 20 nt, synthetic construct
  misc_feature 1..20: note = SEQ ID NO: 2;
  source 1..20: mol_type = other RNA
ccaggtgtctttcaaagtag

SEQ ID NO: 3 - RNA, 76 nt, synthetic construct
  misc_feature 1..76: note = SEQ ID NO: 3;
  source 1..76: mol_type = other RNA
gttttagagctagaaatagcaagttaaaataaggctagtccgttatcaacttgaaaaagtggcaccgagtcggtgc

SEQ ID NO: 4 - RNA, 12 nt, synthetic construct
  misc_feature 1..12: note = SEQ ID NO: 4;
  source 1..12: mol_type = other RNA
agcctccgactt

SEQ ID NO: 5 - RNA, 15 nt, synthetic construct
  misc_feature 1..15: note = SEQ ID NO: 5;
  source 1..15: mol_type = other RNA
ctttgaaagacacct

SEQ ID NO: 6 - RNA, 132 nt, synthetic construct
  misc_feature 1..132: note = SEQ ID NO: 6;
  source 1..132: mol_type = other RNA
ccaggtgtctttcaaagtaggttttagagctagaaatagcaagttaaaataaggctagtccgttatcaacttgaaaaagtggcaccgagtcggtgcacatctcccaagcctccgttctttgaaagacacctg

SEQ ID NO: 7 - RNA, 20 nt, synthetic construct
  misc_feature 1..20: note = SEQ ID NO: 7;
  source 1..20: mol_type = other RNA
ccaggtgtctttcaaagtag

SEQ ID NO: 8 - RNA, 76 nt, synthetic construct; identical to SEQ ID NO: 3
  misc_feature 1..76: note = SEQ ID NO: 8;
  source 1..76: mol_type = other RNA

SEQ ID NO: 9 - RNA, 20 nt, synthetic construct
  misc_feature 1..20: note = SEQ ID NO: 9;
  source 1..20: mol_type = other RNA
acatctcccaagcctccgtt

SEQ ID NO: 10 - RNA, 15 nt, synthetic construct
  misc_feature 1..15: note = SEQ ID NO: 10;
  source 1..15: mol_type = other RNA
ctttgaaagacacct

SEQ ID NO: 11 - RNA, 131 nt, synthetic construct
  misc_feature 1..131: note = SEQ ID NO: 11;
  source 1..131: mol_type = other RNA
ccaggtgtctttcaaagtaggttttagagctagaaatagcaagttaaaataaggctagtccgttatcaacttgaaaaagtggcaccgagtcggtgctctcccaagcctccgacttctttgaaagacacctg

SEQ ID NO: 12 - RNA, 20 nt, synthetic construct
  misc_feature 1..20: note = SEQ ID NO: 12;
  source 1..20: mol_type = other RNA
ccaggtgtctttcaaagtag

SEQ ID NO: 13 - RNA, 76 nt, synthetic construct; identical to SEQ ID NO: 3
  misc_feature 1..76: note = SEQ ID NO: 13;
  source 1..76: mol_type = other RNA

SEQ ID NO: 14 - RNA, 19 nt, synthetic construct
  misc_feature 1..19: note = SEQ ID NO: 14;
  source 1..19: mol_type = other RNA
tctcccaagcctccgactt

SEQ ID NO: 15 - RNA, 15 nt, synthetic construct
  misc_feature 1..15: note = SEQ ID NO: 15;
  source 1..15: mol_type = other RNA
ctttgaaagacacct

SEQ ID NO: 16 - RNA, 129 nt, synthetic construct
  misc_feature 1..129: note = SEQ ID NO: 16;
  source 1..129: mol_type = other RNA
ccaggtgtctttcaaagtaggttttagagctagaaatagcaagttaaaataaggctagtccgttatcaacttgaaaaagtggcaccgagtcggtgctcccaagcctccgacttctttgaaagacacctg

SEQ ID NO: 17 - RNA, 20 nt, synthetic construct
  misc_feature 1..20: note = SEQ ID NO: 17;
  source 1..20: mol_type = other RNA
ccaggtgtctttcaaagtag

SEQ ID NO: 18 - RNA, 76 nt, synthetic construct; identical to SEQ ID NO: 3
  misc_feature 1..76: note = SEQ ID NO: 18;
  source 1..76: mol_type = other RNA

SEQ ID NO: 19 - RNA, 17 nt, synthetic construct
  misc_feature 1..17: note = SEQ ID NO: 19;
  source 1..17: mol_type = other RNA
tcccaagcctccgactt

SEQ ID NO: 20 - RNA, 15 nt, synthetic construct
  misc_feature 1..15: note = SEQ ID NO: 20;
  source 1..15: mol_type = other RNA
ctttgaaagacacct

SEQ ID NO: 21 - RNA, 131 nt, synthetic construct
  misc_feature 1..131: note = SEQ ID NO: 21;
  source 1..131: mol_type = other RNA
ccaggtgtctttcaaagtaggttttagagctagaaatagcaagttaaaataaggctagtccgttatcaacttgaaaaagtggcaccgagtcggtgcatctcccaagcctccgacttctttgaaagacacct

SEQ ID NO: 22 - RNA, 20 nt, synthetic construct
  misc_feature 1..20: note = SEQ ID NO: 22;
  source 1..20: mol_type = other RNA
ccaggtgtctttcaaagtag

SEQ ID NO: 23 - RNA, 76 nt, synthetic construct; identical to SEQ ID NO: 3
  misc_feature 1..76: note = SEQ ID NO: 23;
  source 1..76: mol_type = other RNA

SEQ ID NO: 24 - RNA, 20 nt, synthetic construct
  misc_feature 1..20: note = SEQ ID NO: 24;
  source 1..20: mol_type = other RNA
atctcccaagcctccgactt

SEQ ID NO: 25 - RNA, 15 nt, synthetic construct
  misc_feature 1..15: note = SEQ ID NO: 25;
  source 1..15: mol_type = other RNA
ctttgaaagacacct

SEQ ID NO: 26 - DNA, 123 bp, synthetic construct
  misc_feature 1..123: note = SEQ ID NO: 26;
  source 1..123: mol_type = other DNA
ccaggtgtctttcaaagtaggttttagagctagaaatagcaagttaaaataaggctagtccgttatcaacttgaaaaagtggcaccgagtcggtgcagcctccgacttctttgaaagacacct

SEQ ID NO: 27 - DNA, 20 bp, synthetic construct
  misc_feature 1..20: note = SEQ ID NO: 27;
  source 1..20: mol_type = other DNA
ccaggtgtctttcaaagtag

SEQ ID NO: 28 - DNA, 64 bp, synthetic construct
  misc_feature 1..64: note = SEQ ID NO: 28;
  source 1..64: mol_type = other DNA
gctagaaatagcaagttaaaataaggctagtccgttatcaacttgaaaaagtggcaccgagtcg

SEQ ID NO: 29 - DNA, 12 bp, synthetic construct
  misc_feature 1..12: note = SEQ ID NO: 29;
  source 1..12: mol_type = other DNA
agcctccgactt

SEQ ID NO: 30 - DNA, 15 bp, synthetic construct
  misc_feature 1..15: note = SEQ ID NO: 30;
  source 1..15: mol_type = other DNA
ctttgaaagacacct

SEQ ID NO: 31 - DNA, 20 bp, synthetic construct
  misc_feature 1..20: note = SEQ ID NO: 31;
  source 1..20: mol_type = other DNA
ctccctccaagttgtccacg

SEQ ID NO: 32 - protein, 485 aa, synthetic construct
  REGION 1..485: note = SEQ ID NO: 32;
MALPVALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1458 bp, synthetic construct
  misc_feature 1..1458: note = SEQ ID NO: 33;
  source 1..1458: mol_type = other DNA
atggccttaccagtggccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 34 - protein, 488 aa, synthetic construct
  REGION 1..488: note = SEQ ID NO: 34;
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1464 bp, synthetic construct
  misc_feature 1..1464: note = SEQ ID NO: 35;
  source 1..1464: mol_type = other DNA
atggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - protein, 495 aa, synthetic construct
  REGION 1..495: note = SEQ ID NO: 36;
MASPLTRFLSLNLLLLGESIILGSGEADIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSLQKVNSTTTKPVLRTPSPVHPTGTSQPQRPEDCRPRGSVKGTGLDFACDIYFWALVVVAGVLFCYGLLVTVALCVIWTNSRRNRLLQSDYMNMTPRRPGLTRKPYQPYAPARDFAAYRPKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 37 - DNA, 1485 bp, synthetic construct
  misc_feature 1..1485: note = SEQ ID NO: 37;
  source 1..1485: mol_type = other DNA
atggcctcacccctgaccagattcctgtcactgaacctgctgctgctgggcgagtcaatcatcctgggctcaggcgaggccgacatccagatgacccagagccctgccagcctgtctaccagcctgggcgagacagtgaccatccagtgtcaggccagcgaggacatctactctggcctggcttggtatcagcagaagcccggcaagagccctcagctgctgatctacggcgccagcgacctgcaggacggcgtgcctagcagattcagcggcagcggctccggaacccagtacagcctgaagatcaccagcatgcagaccgaggacgagggcgtgtacttctgccagcaaggcctgacctaccctagaaccttcggaggaggcaccaagctggaactgaagggcggaggcggaagtggaggcggaggatctggcggcggaggctctgaagtgcagctgcagcagtctggcgctgaactggtccggcctggcactagcgtgaagctgtcctgcaaggtgtccggcgacaccatcaccttctactacatgcacttcgtgaagcagaggccaggacagggcctggaatggatcggcagaatcgaccctgaggacgagagcaccaagtacagcgagaagttcaagaacaaggccaccctgaccgccgacaccagcagcaacaccgcctacctgaagctgtctagcctgacctccgaggacaccgccacctacttttgcatctacggcggctactacttcgactactggggccagggcgtgatggtcaccgtgtccagcctgcagaaggtgaactcaaccaccaccaagcccgtgctgagaaccccctcacccgtgcaccccaccggcacctcacagccccagagacccgaggactgcagacccagaggctcagtgaagggcaccggcctggacttcgcctgcgacatctacttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccaagttctcaagatcagccgagaccgccgccaacctgcaggaccccaaccagctgtacaacgagctgaacctgggcagaagagaggagtacgacgtgctggagaagaagagagccagagaccccgagatgggcggcaagcagcagagaagaagaaacccccaggagggcgtgtacaacgccctgcagaaggacaagatggccgaggcctactcagagatcggcaccaagggcgagagaagaagaggcaagggccacgacggcctgtaccagggcctgtcaaccgccaccaaggacacctacgacgccctgcacatgcagaccctggcccccaga

SEQ ID NO: 38 - protein, 501 aa, synthetic construct
  REGION 1..501: note = SEQ ID NO: 38;
MASPLTRFLSLNLLLLGESIILGSGEAAQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARFDYGDFYDAFDIWGQGTMVTVSSGGGGSGGGGSGGGGSEIVLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKRLQKVNSTTTKPVLRTPSPVHPTGTSQPQRPEDCRPRGSVKGTGLDFACDIYIWAPLAGICVALLLSLIITLIKWIRKKFPHIFKQPFKKTTGAAQEEDACSCRCPQEEEGGGGGYELKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 39 - DNA, 1503 bp, synthetic construct
  misc_feature 1..1503: note = SEQ ID NO: 39;
  source 1..1503: mol_type = other DNA
atggccagccccctgaccagattcctgagcctgaacctgctgctgctgggcgagagcatcatcctgggcagcggcgaggccgcccaggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtccaagaaccagttctccctgaagctgagctctgtgaccgctgcggacacggccgtgtattactgtgcgagatttgactacggtgacttctatgatgcttttgatatctggggccaagggacaatggtcaccgtctcttcaggtggtggtggtagcggcggcggcggctctggtggtggtggatccgaaattgtgttgacgcagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgtctgcagaaggtgaacagcaccaccaccaagcccgtgctgagaacccccagccccgtgcaccccaccggcaccagccagccccagagacccgaggactgcagacccagaggcagcgtgaagggcaccggcctggacttcgcctgcgacatctacatctgggcccccctggccggcatctgcgtggccctgctgctgagcctgatcatcaccctgatcaagtggatcagaaagaagttcccccacatcttcaagcagcccttcaagaagaccaccggcgccgcccaggaggaggacgcctgcagctgcagatgcccccaggaggaggagggcggcggcggcggctacgagctgaagttcagcagaagcgccgagaccgccgccaacctgcaggaccccaaccagctgtacaacgagctgaacctgggcagaagagaggagtacgacgtgctggagaagaagagagccagagaccccgagatgggcggcaagcagcagagaagaagaaacccccaggagggcgtgtacaacgccctgcagaaggacaagatggccgaggcctacagcgagatcggcaccaagggcgagagaagaagaggcaagggccacgacggcctgtaccagggcctgagcaccgccaccaaggacacctacgacgccctgcacatgcagaccctggcccccaga

SEQ ID NO: 40 - protein, 485 aa, synthetic construct
  REGION 1..485: note = SEQ ID NO: 40;
MALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 1455 bp, synthetic construct
  misc_feature 1..1455: note = SEQ ID NO: 41;
  source 1..1455: mol_type = other DNA
atggccctccctgtcaccgccctgctgcttccgctggctcttctgctccacgccgctcggccccaagtccaactcgttcaatcaggcgcagaagtcgaaaagcccggagcatcagtcaaagtctcttgcaaggcttccggctacaccttcacggactactacatgcactgggtgcgccaggctccaggccagggactggagtggatgggatggatcaacccgaattccgggggaactaactacgcccagaagtttcagggccgggtgactatgactcgcgatacctcgatctcgactgcgtacatggagctcagccgcctccggtcggacgataccgccgtgtactattgtgcgtcgggatgggacttcgactactgggggcagggcactctggtcactgtgtcaagcggaggaggtggatcaggtggaggtggaagcgggggaggaggttccggcggcggaggatcagatatcgtgatgacgcaatcgccttcctcgttgtccgcatccgtgggagacagggtgaccattacttgcagagcgtcccagtccattcggtactacctgtcgtggtaccagcagaagccggggaaagccccaaaactgcttatctatactgcctcgatcctccaaaacggcgtgccatcaagattcagcggttcgggcagcgggaccgactttaccctgactatcagcagcctgcagccggaagatttcgccacgtactactgcctgcaaacctacaccaccccggacttcggacctggaaccaaggtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 42 - DNA, 738 bp, synthetic construct
  misc_feature 1..738: note = SEQ ID NO: 42;
  source 1..738: mol_type = other DNA
gcccaggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtccaagaaccagttctccctgaagctgagctctgtgaccgctgcggacacggccgtgtattactgtgcgagatttgactacggtgacttctatgatgcttttgatatctggggccaagggacaatggtcaccgtctcttcaggtggtggtggtagcggcggcggcggctctggtggtggtggatccgaaattgtgttgacgcagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 43 - protein, 246 aa, synthetic construct
  REGION 1..246: note = SEQ ID NO: 43;
AQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARFDYGDFYDAFDIWGQGTMVTVSSGGGGSGGGGSGGGGSEIVLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKR

SEQ ID NO: 44 - DNA, 723 bp, synthetic construct
  misc_feature 1..723: note = SEQ ID NO: 44;
  source 1..723: mol_type = other DNA
caagtccaactcgttcaatcaggcgcagaagtcgaaaagcccggagcatcagtcaaagtctcttgcaaggcttccggctacaccttcacggactactacatgcactgggtgcgccaggctccaggccagggactggagtggatgggatggatcaacccgaattccgggggaactaactacgcccagaagtttcagggccgggtgactatgactcgcgatacctcgatctcgactgcgtacatggagctcagccgcctccggtcggacgataccgccgtgtactattgtgcgtcgggatgggacttcgactactgggggcagggcactctggtcactgtgtcaagcggaggaggtggatcaggtggaggtggaagcgggggaggaggttccggcggcggaggatcagatatcgtgatgacgcaatcgccttcctcgttgtccgcatccgtgggagacagggtgaccattacttgcagagcgtcccagtccattcggtactacctgtcgtggtaccagcagaagccggggaaagccccaaaactgcttatctatactgcctcgatcctccaaaacggcgtgccatcaagattcagcggttcgggcagcgggaccgactttaccctgactatcagcagcctgcagccggaagatttcgccacgtactactgcctgcaaacctacaccaccccggacttcggacctggaaccaaggtggagatcaag

SEQ ID NO: 45 - protein, 241 aa, synthetic construct
  REGION 1..241: note = SEQ ID NO: 45;
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 46 - DNA, 810 bp, synthetic construct
  misc_feature 1..810: note = SEQ ID NO: 46;
  source 1..810: mol_type = other DNA
atggccttaccagtgaccgccttgctcctgccgctggccttgctgctccacgccgccaggccgggatccgatgttgtgatgacccaaactccactctccctgcctgtcagtcttggagatcaagcctccatctcttgcagatctagtcagagtcttgtacaccgtaacggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacacgttcctccgctcacgttcggtgctgggaccaagctggagctgaaaggaggtggcgggtcagggggtggcggaagcggaggcggcggttcaggcggaggaggctcggaggtgcagcttctgcagtctggacctgagctggagaagccttccgcttcagtgatgatatcctgcaaggcttctggttcctccttcactggctacaacatgaactgggtgaggcagaatattggaaagagccttgaatggattggagctattgatccttactacggtggaactagctacaaccagaagttcaagggcagggccacattgactgtagacaaatcgtccagcacagcctacatgcacctcaagagcctgacatctgaggactctgtctattactgtgtaagcggaatggagtactggggtcaaggaacctcagtcaccgtctcctcatccgga

SEQ ID NO: 47 - protein, 270 aa, synthetic construct
  REGION 1..270: note = SEQ ID NO: 47;
MALPVTALLLPLALLLHAARPGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSGGGGSEVQLLQSGPELEKPSASVMISCKASGSSFTGYNMNWVRQNIGKSLEWIGAIDPYYGGTSYNQKFKGRATLTVDKSSSTAYMHLKSLTSEDSVYYCVSGMEYWGQGTSVTVSSSG

SEQ ID NO: 48 - DNA, 798 bp, synthetic construct
  misc_feature 1..798: note = SEQ ID NO: 48;
  source 1..798: mol_type = other DNA
atggattttcaggtgcagattttcagcttcctgctaatcagtgcctcagtcataatgtccagaggagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctttattctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaacgcactgggtctacatctggatctgggaagccgggttctggtgagggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcg

SEQ ID NO: 49 - protein, 266 aa, synthetic construct
  REGION 1..266: note = SEQ ID NO: 49;
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 50 - DNA, 798 bp, synthetic construct
  misc_feature 1..798: note = SEQ ID NO: 50;
  source 1..798: mol_type = other DNA
atggattttcaggtgcagattttcagcttcctgctaatcagtgcctcagtcataatgtccagaggagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttccttgagtctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaacgcactgggtctacatctggatctgggaagccgggttctggtgagggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttcgttgctatggacgtgtggggtcaaggaaccctggtcaccgtctcctcg

SEQ ID NO: 51 - protein, 266 aa, synthetic construct
  REGION 1..266: note = SEQ ID NO: 51;
MDFQVQIFSFLLISASVIMSRGDIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFVAMDVWGQGTLVTVSS

SEQ ID NO: 52 - DNA, 732 bp, synthetic construct
  misc_feature 1..732: note = SEQ ID NO: 52;
  source 1..732: mol_type = other DNA
caggtgcagctgcagcagtctgatgccgagctcgtgaagcctggcagcagcgtgaagatcagctgcaaggccagcggctacaccttcaccgaccacgccatccactgggtcaagcagaagcctgagcagggcctggagtggatcggccacttcagccccggcaacaccgacatcaagtacaacgacaagttcaagggcaaggccaccctgaccgtggacagaagcagcagcaccgcctacatgcagctgaacagcctgaccagcgaggacagcgccgtgtacttctgcaagaccagcaccttctttttcgactactggggccagggcacaaccctgacagtgtctagcggaggcggaggatctggcggcggaggaagtggcggagggggatctgaactcgtgatgacccagagccccagctctctgacagtgacagccggcgagaaagtgaccatgatctgcaagtcctcccagagcctgctgaactccggcgaccagaagaactacctgacctggtatcagcagaaacccggccagccccccaagctgctgatcttttgggccagcacccgggaaagcggcgtgcccgatagattcacaggcagcggctccggcaccgactttaccctgaccatcagctccgtgcaggccgaggacctggccgtgtattactgccagaacgactacagctaccccctgaccttcggagccggcaccaagctggaactgaag

SEQ ID NO: 53 - protein, 244 aa, synthetic construct
  REGION 1..244: note = SEQ ID NO: 53;
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELK

SEQ ID NO: 54 - DNA, 792 bp, synthetic construct
  misc_feature 1..792: note = SEQ ID NO: 54;
  source 1..792: mol_type = other DNA
atggccctgcctgtgacagccctgctgctgcctctggctctgctgctgcacgccgccagacctggatctgacattgtgatgacccagtctcacaaattcatgtccacatcagtaggagacagggtcagcatcatctgtaaggccagtcaagatgtgggtactgctgtagactggtatcaacagaaaccaggacaatctcctaaactactgatttattgggcatccactcggcacactggagtccctgatcgcttcacaggcagtggatctgggacagacttcactctcaccattactaacgttcagtctgaagacttggcagattatttctgtcagcaatataacagctatcctctcacgttcggtgctgggaccatgctggacctgaaaggaggcggaggatctggcggcggaggaagttctggcggaggcagcgaggtgcagctgcagcagagcggacccgagctcgtgaagcctggaacaagcgtgcggatcagctgcaagaccagcggctacaccttcaccgagtacaccatccactgggtcaagcagtcccacggcaagagcctggagtggatcggcaatatcaaccccaacaacggcggcaccacctacaaccagaagttcgaggacaaggccaccctgaccgtggacaagagcagcagcaccgcctacatggaactgcggagcctgaccagcgaggacagcgccgtgtactattgtgccgccggttggaacttcgactactggggccagggcacaaccctgacagtgtctagcgctagctccgga

SEQ ID NO: 55 - protein, 264 aa, synthetic construct
  REGION 1..264: note = SEQ ID NO: 55;
MALPVTALLLPLALLLHAARPGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKGGGGSGGGGSSGGGSEVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSASSG

SEQ ID NO: 56 - DNA, 801 bp, synthetic construct
  misc_feature 1..801: note = SEQ ID NO: 56;
  source 1..801: mol_type = other DNA
atggccctccctgtcaccgccctgctgcttccgctggctcttctgctccacgccgctcggcccgagattcagctcgtgcaatcgggagcggaagtcaagaagccaggagagtccttgcggatctcatgcaagggtagcggctttaacatcgaggattactacatccactgggtgaggcagatgccggggaagggactcgaatggatgggacggatcgacccagaaaacgacgaaactaagtacggtccgatcttccaaggccatgtgactattagcgccgatacttcaatcaataccgtgtatctgcaatggtcctcattgaaagcctcagataccgcgatgtactactgtgctttcagaggaggggtctactggggacagggaactaccgtgactgtctcgtccggcggaggcgggtcaggaggtggcggcagcggaggaggagggtccggcggaggtgggtccgacgtcgtgatgacccagagccctgacagcctggcagtgagcctgggcgaaagagctaccattaactgcaaatcgtcgcagagcctgctggactcggacggaaaaacgtacctcaattggctgcagcaaaagcctggccagccaccgaagcgccttatctcactggtgtcgaagctggattcgggagtgcccgatcgcttctccggctcgggatcgggtactgacttcaccctcactatctcctcgcttcaagcagaggacgtggccgtctactactgctggcagggaacccactttccgggaaccttcggcggagggacgaaagtggagatcaag

SEQ ID NO: 57 - protein, 267 aa, synthetic construct
  REGION 1..267: note = SEQ ID NO: 57;
MALPVTALLLPLALLLHAARPEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 58 - DNA, 729 bp, synthetic construct
  misc_feature 1..729: note = SEQ ID NO: 58;
  source 1..729: mol_type = other DNA
caaattgttctcacccagtctccagcgctcatgtctgcttctccaggggagaaggtcaccatgacctgcactgccagctcaagtgttagttacatgtactggtaccagcagaagccacgatcctcccccaaaccctggatttttctcacctccaacctggcttctggagtccctgctcgcttcagtggccgtgggtctgggacctctttctctctcacaatcagcagcatggaggctgaagatgctgccacttattactgccagcagtggagtggttacccacccatcacattcggctcggggacaaagttggaaataaaaggtggaggtggcagcggaggaggtgggtccggcggtggaggaagccaggtccaactgcagcagcctggggctgaactggtaaagcctggggcttcagtgaagttgtcctgcaaggcgtctggctacaccatcaccagctactctctgcactgggtgaagcagaggcctggacaaggccttgagtggattggagagattaatcctgccaatggtgatcataacttcagtgagaagttcgagatcaaggccacactgactgtagacagctcctccaacacagcattcatgcaactcagcaggctgacatctgaggactctgcggtctattactgtacaagattggacgatagtaggttccactggtacttcgatgtctggggcgcagggaccacggtcaccgtctcctca

SEQ ID NO: 59 - protein, 243 aa, synthetic construct
  REGION 1..243: note = SEQ ID NO: 59;
QIVLTQSPALMSASPGEKVTMTCTASSSVSYMYWYQQKPRSSPKPWIFLTSNLASGVPARFSGRGSGTSFSLTISSMEAEDAATYYCQQWSGYPPITFGSGTKLEIKGGGGSGGGGSGGGGSQVQLQQPGAELVKPGASVKLSCKASGYTITSYSLHWVKQRPGQGLEWIGEINPANGDHNFSEKFEIKATLTVDSSSNTAFMQLSRLTSEDSAVYYCTRLDDSRFHWYFDVWGAGTTVTVSS

SEQ ID NO: 60 - protein, 27 aa, synthetic construct
  REGION 1..27: note = SEQ ID NO: 60;
MASPLTRFLSLNLLLLGESIILGSGEA

SEQ ID NO: 61 - DNA, 81 bp, synthetic construct
  misc_feature 1..81: note = SEQ ID NO: 61;
  source 1..81: mol_type = other DNA
atggcctcacccctgaccagattcctgtcactgaacctgctgctgctgggcgagtcaatcatcctgggctcaggcgaggcc

SEQ ID NO: 62 - protein, 238 aa, synthetic construct
  REGION 1..238: note = SEQ ID NO: 62;
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

SEQ ID NO: 63 - DNA, 714 bp, synthetic construct
  misc_feature 1..714: note = SEQ ID NO: 63;
  source 1..714: mol_type = other DNA
gacatccagatgacccagagccctgccagcctgtctaccagcctgggcgagacagtgaccatccagtgtcaggccagcgaggacatctactctggcctggcttggtatcagcagaagcccggcaagagccctcagctgctgatctacggcgccagcgacctgcaggacggcgtgcctagcagattcagcggcagcggctccggaacccagtacagcctgaagatcaccagcatgcagaccgaggacgagggcgtgtacttctgccagcaaggcctgacctaccctagaaccttcggaggaggcaccaagctggaactgaagggcggaggcggaagtggaggcggaggatctggcggcggaggctctgaagtgcagctgcagcagtctggcgctgaactggtccggcctggcactagcgtgaagctgtcctgcaaggtgtccggcgacaccatcaccttctactacatgcacttcgtgaagcagaggccaggacagggcctggaatggatcggcagaatcgaccctgaggacgagagcaccaagtacagcgagaagttcaagaacaaggccaccctgaccgccgacaccagcagcaacaccgcctacctgaagctgtctagcctgacctccgaggacaccgccacctacttttgcatctacggcggctactacttcgactactggggccagggcgtgatggtcaccgtgtccagc

SEQ ID NO: 64 - protein, 51 aa, synthetic construct
  REGION 1..51: note = SEQ ID NO: 64;
LQKVNSTTTKPVLRTPSPVHPTGTSQPQRPEDCRPRGSVKGTGLDFACDIY

SEQ ID NO: 65 - DNA, 153 bp, synthetic construct
  misc_feature 1..153: note = SEQ ID NO: 65;
  source 1..153: mol_type = other DNA
ctgcagaaggtgaactcaaccaccaccaagcccgtgctgagaaccccctcacccgtgcaccccaccggcacctcacagccccagagacccgaggactgcagacccagaggctcagtgaagggcaccggcctggacttcgcctgcgacatctac

SEQ ID NO: 66 - protein, 27 aa, synthetic construct
  REGION 1..27: note = SEQ ID NO: 66;
FWALVVVAGVLFCYGLLVTVALCVIWT

SEQ ID NO: 67 - DNA, 81 bp, synthetic construct
  misc_feature 1..81: note = SEQ ID NO: 67;
  source 1..81: mol_type = other DNA
ttttgggcactggtcgtggttgctggagtcctgttttgttatggcttgctagtgacagtggctctttgtgttatctggaca

SEQ ID NO: 68 - protein, 41 aa, synthetic construct
  REGION 1..41: note = SEQ ID NO: 68;
NSRRNRLLQSDYMNMTPRRPGLTRKPYQPYAPARDFAAYRP

SEQ ID NO: 69 - DNA, 123 bp, synthetic construct
  misc_feature 1..123: note = SEQ ID NO: 69;
  source 1..123: mol_type = other DNA
aatagtagaaggaacagactccttcaaagtgactacatgaacatgactccccggaggcctgggctcactcgaaagccttaccagccctacgcccctgccagagactttgcagcgtaccgcccc

SEQ ID NO: 70 - protein, 111 aa, synthetic construct
  REGION 1..111: note = SEQ ID NO: 70;
KFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 71 - DNA, 333 bp, synthetic construct
  misc_feature 1..333: note = SEQ ID NO: 71;
  source 1..333: mol_type = other DNA
aagttctcaagatcagccgagaccgccgccaacctgcaggaccccaaccagctgtacaacgagctgaacctgggcagaagagaggagtacgacgtgctggagaagaagagagccagagaccccgagatgggcggcaagcagcagagaagaagaaacccccaggagggcgtgtacaacgccctgcagaaggacaagatggccgaggcctactcagagatcggcaccaagggcgagagaagaagaggcaagggccacgacggcctgtaccagggcctgtcaaccgccaccaaggacacctacgacgccctgcacatgcagaccctggcccccaga

SEQ ID NO: 72 - protein, 21 aa, synthetic construct
  REGION 1..21: note = SEQ ID NO: 72;
MALPVTALLLPLALLLHAARP

SEQ ID NO: 73 - DNA, 63 bp, synthetic construct
  misc_feature 1..63: note = SEQ ID NO: 73;
  source 1..63: mol_type = other DNA
atggccttaccagtgaccgccttgctcctgccgctggccttgctgctccacgccgccaggccg

SEQ ID NO: 74 - protein, 242 aa, synthetic construct
  REGION 1..242: note = SEQ ID NO: 74;
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 75 - DNA, 726 bp, synthetic construct
  misc_feature 1..726: note = SEQ ID NO: 75;
  source 1..726: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagc

SEQ ID NO: 76 - protein, 45 aa, synthetic construct
  REGION 1..45: note = SEQ ID NO: 76;
TTTPAPRPPTPAPTIASQPLSLRPEACRPAAGGAVHTRGLDFACD

SEQ ID NO: 77 - DNA, 135 bp, synthetic construct
  misc_feature 1..135: note = SEQ ID NO: 77;
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 78 - protein, 27 aa, synthetic construct
  REGION 1..27: note = SEQ ID NO: 78;
FWVLVVVGGVLACYSLLVTVAFIIFWV

SEQ ID NO: 79 - DNA, 81 bp, synthetic construct
  misc_feature 1..81: note = SEQ ID NO: 79;
  source 1..81: mol_type = other DNA
ttttgggtgctggtggtggttggtggagtcctggcttgctatagcttgctagtaacagtggcctttattattttctgggtg

SEQ ID NO: 80 - protein, 41 aa, synthetic construct
  REGION 1..41: note = SEQ ID NO: 80;
RSKRSRLLHSDYMNMTPRRPGPTRKHYQPYAPPRDFAAYRS

SEQ ID NO: 81 - DNA, 123 bp, synthetic construct
  misc_feature 1..123: note = SEQ ID NO: 81;
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 82 - protein, 112 aa, synthetic construct
  REGION 1..112: note = SEQ ID NO: 82;
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - DNA, 336 bp, synthetic construct
  misc_feature 1..336: note = SEQ ID NO: 83;
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 84 - protein, 27 aa, synthetic construct
  REGION 1..27: note = SEQ ID NO: 84;
MASPLTRFLSLNLLLLGESIILGSGEA

SEQ ID NO: 85 - DNA, 81 bp, synthetic construct
  misc_feature 1..81: note = SEQ ID NO: 85;
  source 1..81: mol_type = other DNA
atggccagccccctgaccagattcctgagcctgaacctgctgctgctgggcgagagcatcatcctgggcagcggcgaggcc

SEQ ID NO: 86 - protein, 246 aa, synthetic construct; identical to SEQ ID NO: 43
  REGION 1..246: note = SEQ ID NO: 86;

SEQ ID NO: 87 - DNA, 738 bp, synthetic construct; identical to SEQ ID NO: 42
  misc_feature 1..738: note = SEQ ID NO: 87;
  source 1..738: mol_type = other DNA

SEQ ID NO: 88 - protein, 51 aa, synthetic construct; identical to SEQ ID NO: 64
  REGION 1..51: note = SEQ ID NO: 88;

SEQ ID NO: 89 - DNA, 153 bp, synthetic construct
  misc_feature 1..153: note = SEQ ID NO: 89;
  source 1..153: mol_type = other DNA
ctgcagaaggtgaacagcaccaccaccaagcccgtgctgagaacccccagccccgtgcaccccaccggcaccagccagccccagagacccgaggactgcagacccagaggcagcgtgaagggcaccggcctggacttcgcctgcgacatctac

SEQ ID NO: 90 - protein, 21 aa, synthetic construct
  REGION 1..21: note = SEQ ID NO: 90;
IWAPLAGICVALLLSLIITLI

SEQ ID NO: 91 - DNA, 63 bp, synthetic construct
  misc_feature 1..63: note = SEQ ID NO: 91;
  source 1..63: mol_type = other DNA
atctgggcccccctggccggcatctgcgtggccctgctgctgagcctgatcatcaccctgatc

SEQ ID NO: 92 - protein, 45 aa, synthetic construct
  REGION 1..45: note = SEQ ID NO: 92;
KWIRKKFPHIFKQPFKKTTGAAQEEDACSCRCPQEEEGGGGGYEL

SEQ ID NO: 93 - DNA, 135 bp, synthetic construct
  misc_feature 1..135: note = SEQ ID NO: 93;
  source 1..135: mol_type = other DNA
aagtggatcagaaagaagttcccccacatcttcaagcagcccttcaagaagaccaccggcgccgcccaggaggaggacgcctgcagctgcagatgcccccaggaggaggagggcggcggcggcggctacgagctg

SEQ ID NO: 94 - protein, 111 aa, synthetic construct; identical to SEQ ID NO: 70
  REGION 1..111: note = SEQ ID NO: 94;

SEQ ID NO: 95 - DNA, 333 bp, synthetic construct
  misc_feature 1..333: note = SEQ ID NO: 95;
  source 1..333: mol_type = other DNA
aagttcagcagaagcgccgagaccgccgccaacctgcaggaccccaaccagctgtacaacgagctgaacctgggcagaagagaggagtacgacgtgctggagaagaagagagccagagaccccgagatgggcggcaagcagcagagaagaagaaacccccaggagggcgtgtacaacgccctgcagaaggacaagatggccgaggcctacagcgagatcggcaccaagggcgagagaagaagaggcaagggccacgacggcctgtaccagggcctgagcaccgccaccaaggacacctacgacgccctgcacatgcagaccctggcccccaga

SEQ ID NO: 96 - protein, 21 aa, synthetic construct
  REGION 1..21: note = SEQ ID NO: 96;
MALPVTALLLPLALLLHAARP

SEQ ID NO: 97 - DNA, 63 bp, synthetic construct
  misc_feature 1..63: note = SEQ ID NO: 97;
  source 1..63: mol_type = other DNA
atggccctccctgtcaccgccctgctgcttccgctggctcttctgctccacgccgctcggccc

SEQ ID NO: 98 - protein, 241 aa, synthetic construct; identical to SEQ ID NO: 45
  REGION 1..241: note = SEQ ID NO: 98;

SEQ ID NO: 99 - DNA, 723 bp, synthetic construct; identical to SEQ ID NO: 44
  misc_feature 1..723: note = SEQ ID NO: 99;
  source 1..723: mol_type = other DNA

SEQ ID NO: 100 - protein, 45 aa, synthetic construct; identical to SEQ ID NO: 76
  REGION 1..45: note = SEQ ID NO: 100;

SEQ ID NO: 101 - DNA, 135 bp, synthetic construct
  misc_feature 1..135: note = SEQ ID NO: 101;
  source 1..135: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 102 - protein, 24 aa, synthetic construct
  REGION 1..24: note = SEQ ID NO: 102;
IYIWAPLAGTCGVLLLSLVITLYC

SEQ ID NO: 103 - DNA, 72 bp, synthetic construct
  misc_feature 1..72: note = SEQ ID NO: 103;
  source 1..72: mol_type = other DNA
atctacatttgggcccctctggctggtacttgcggggtcctgctgctttcactcgtgatcactctttactgt

SEQ ID NO: 104 - protein, 42 aa, synthetic construct
  REGION 1..42: note = SEQ ID NO: 104;
KRGRKKLLYIFKQPFMRPVQTTQEEDGCSCRFPEEEEGGCEL

SEQ ID NO: 105 - DNA, 126 bp, synthetic construct
  misc_feature 1..126: note = SEQ ID NO: 105;
  source 1..126: mol_type = other DNA
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 106 - protein, 112 aa, synthetic construct
  REGION 1..112: note = SEQ ID NO: 106;
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - DNA, 336 bp, synthetic construct
  misc_feature 1..336: note = SEQ ID NO: 107;
  source 1..336: mol_type = other DNA
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 108-109 - skipped (2 entries)

SEQ ID NO: 110 - DNA, 155 bp, synthetic construct
  misc_feature 1..155: note = SEQ ID NO: 110;
  misc_feature 3: note = n is a, c, g, or t
  source 1..155: mol_type = other DNA
ttngtgtagttttatgacaaagaaaattttctgagttacttttgtatccccacccccttaaagaaaggaggaactcacagtaacctcaactcctgcaacaacagatggccttaccagtggccttgctcctgccgctggccttgctgctccacgcc

SEQ ID NO: 111 - DNA, 163 bp, synthetic construct
  misc_feature 1..163: note = SEQ ID NO: 111;
  misc_feature 3: note = n is a, c, g, or t
  source 1..163: mol_type = other DNA
gantgggaagacaatagcaggcatgctggggatgcggtgggctctatggtgtacaggatgcaactcctgtcttgcattgcaaaatttgcacagatgggactaatagcagctcatctgaggtaaagagtaactttaatttgtttttttgaaaacccaagtttga

SEQ ID NO: 112 - DNA, 78 bp, synthetic construct
  misc_feature 1..78: note = SEQ ID NO: 112;
  source 1..78: mol_type = other DNA
atcactactcacagtaacctcaactcctgccacaatgtacaggatgcaactcctgtcttgcattgcactaagtcttgc

SEQ ID NO: 113 - protein, 15 aa, synthetic construct
  REGION 1..15: note = SEQ ID NO: 113;
MYRMQLLSCIALSLA

SEQ ID NO: 114 - DNA, 20 bp, synthetic construct
  misc_feature 1..20: note = SEQ ID NO: 114;
  source 1..20: mol_type = other DNA
caatgtacaggatgcaactc

SEQ ID NO: 115 - DNA, 66 bp, synthetic construct
  misc_feature 1..66: note = SEQ ID NO: 115;
  source 1..66: mol_type = other DNA
atcactactcacagtaacctcaactcctgccacaattggaaatgatgcttgtccttgcctgaattg

SEQ ID NO: 116 - DNA, 66 bp, synthetic construct
  misc_feature 1..66: note = SEQ ID NO: 116;
  source 1..66: mol_type = other DNA
atcactactcacagtaacctcaactcctgccacaatgtacaggatgcaactcctgtcttgcattgc

SEQ ID NO: 117 - DNA, 20 bp, synthetic construct
  misc_feature 1..20: note = SEQ ID NO: 117;
  source 1..20: mol_type = other DNA
caatgtacaggatgcaactc

SEQ ID NO: 118 - DNA, 75 bp, synthetic construct
  misc_feature 1..75: note = SEQ ID NO: 118;
  misc_feature 26: note = n is a, c, g, or t
  source 1..75: mol_type = other DNA
acagtaacctcaactcctgccacaantgtacaggatgcaactcctgtcttgcattgcactaagtcttgcacttgt

SEQ ID NO: 119 - DNA, 49 bp, synthetic construct
  misc_feature 1..49: note = SEQ ID NO: 119;
  source 1..49: mol_type = other DNA
acagtaacctcaactcctgtcttgcattgcactaagtcttgcacttgtc

SEQ ID NO: 120 - DNA, 73 bp, synthetic construct
  misc_feature 1..73: note = SEQ ID NO: 120;
  source 1..73: mol_type = other DNA
acagtaacctcaactcctgccacaatacaggatgcaactcctgtcttgcattgcactaagtcttgcacttgtc

SEQ ID NO: 121 - DNA, 74 bp, synthetic construct
  misc_feature 1..74: note = SEQ ID NO: 121;
  source 1..74: mol_type = other DNA
acagtaacctcaactcctgccacaagtacaggatgcaactcctgtcttgcattgcactaagtcttgcacttgtc

SEQ ID NO: 122 - DNA, 71 bp, synthetic construct
  misc_feature 1..71: note = SEQ ID NO: 122;
  source 1..71: mol_type = other DNA
acagtaacctcaactcctgccacaacaggatgcaactcctgtcttgcattgcactaagtcttgcacttgtc

SEQ ID NO: 123 - DNA, 75 bp, synthetic construct
  misc_feature 1..75: note = SEQ ID NO: 123;
  misc_feature 26..27: note = n is a, c, g, or t
  source 1..75: mol_type = other DNA
acagtaacctcaactcctgccacaanntgtacaggatgcaactcctgtcttgcattgcactaagtcttgcacttg

SEQ ID NO: 124 - DNA, 61 bp, synthetic construct
  misc_feature 1..61: note = SEQ ID NO: 124;
  source 1..61: mol_type = other DNA
acagtaacctcaactcctgccacaactcctgtcttgcattgcactaagtcttgcacttgtc

SEQ ID NO: 125 - DNA, 72 bp, synthetic construct
  misc_feature 1..72: note = SEQ ID NO: 125;
  source 1..72: mol_type = other DNA
acagtaacctcaactcctgccacaaacaggatgcaactcctgtcttgcattgcactaagtcttgcacttgtc

SEQ ID NO: 126 - DNA, 75 bp, synthetic construct
  misc_feature 1..75: note = SEQ ID NO: 126;
  misc_feature 26..39: note = n is a, c, g, or t
  source 1..75: mol_type = other DNA
acagtaacctcaactcctgccacaannnnnnnnnnnnnntgtacaggatgcaactcctgtcttgcattgcactaa

SEQ ID NO: 127 - DNA, 75 bp, synthetic construct
  misc_feature 1..75: note = SEQ ID NO: 127;
  misc_feature 26..29: note = n is a, c, g, or t
  source 1..75: mol_type = other DNA
acagtaacctcaactcctgccacaannnntgtacaggatgcaactcctgtcttgcattgcactaagtcttgcact

SEQ ID NO: 128 - DNA, 48 bp, synthetic construct
  misc_feature 1..48: note = SEQ ID NO: 128;
  source 1..48: mol_type = other DNA
acagtaacctcaatcctgtcttgcattgcactaagtcttgcacttgtc

SEQ ID NO: 129 - DNA, 64 bp, synthetic construct
  misc_feature 1..64: note = SEQ ID NO: 129;
  source 1..64: mol_type = other DNA
acagtaacctcaactcctgccacaacaactcctgtcttgcattgcactaagtcttgcacttgtc

SEQ ID NO: 130 - DNA, 67 bp, synthetic construct
  misc_feature 1..67: note = SEQ ID NO: 130;
  source 1..67: mol_type = other DNA
acagtaacctcaactcctgccacaaatgcaactcctgtcttgcattgcactaagtcttgcacttgtc

SEQ ID NO: 131 - DNA, 70 bp, synthetic construct
  misc_feature 1..70: note = SEQ ID NO: 131;
  source 1..70: mol_type = other DNA
acagtaacctcaactcctgccacaaaggatgcaactcctgtcttgcattgcactaagtcttgcacttgtc

SEQ ID NO: 132 - DNA, 75 bp, synthetic construct
  misc_feature 1..75: note = SEQ ID NO: 132;
  source 1..75: mol_type = other DNA
acagtaacctcaactcctgccacaatgtacaggatgcaactcctgtcttgcattgcactaagtcttgcacttgtc

SEQ ID NO: 133 - DNA, 41 bp, synthetic construct
  misc_feature 1..41: note = SEQ ID NO: 133;
  source 1..41: mol_type = other DNA
cattttaaattaaaatagcgcctgccacaacgttacataac

SEQ ID NO: 134 - DNA, 35 bp, synthetic construct
  misc_feature 1..35: note = SEQ ID NO: 134;
  misc_feature 4..6: note = n is a, c, g, or t
  source 1..35: mol_type = other DNA
tttnnngtgggctccttcctgtacatccccagcat

SEQ ID NO: 135 - DNA, 22 bp, synthetic construct
  misc_feature 1..22: note = SEQ ID NO: 135;
  source 1..22: mol_type = other DNA
caagcctcccactactttgaaa

SEQ ID NO: 136 - DNA, 22 bp, synthetic construct
  misc_feature 1..22: note = SEQ ID NO: 136;
  source 1..22: mol_type = other DNA
caagcctcccacttctttgaaa

SEQ ID NO: 137 - DNA, 22 bp, synthetic construct
  misc_feature 1..22: note = SEQ ID NO: 137;
  source 1..22: mol_type = other DNA
caagcctcccactactttgaaa

SEQ ID NO: 138 - DNA, 21 bp, synthetic construct
  misc_feature 1..21: note = SEQ ID NO: 138;
  source 1..21: mol_type = other DNA
ttcaaagtagtgggaggcttg

SEQ ID NO: 139 - DNA, 21 bp, synthetic construct
  misc_feature 1..21: note = SEQ ID NO: 139;
  source 1..21: mol_type = other DNA
ttcaaagaagtgggaggcttg

SEQ ID NO: 140 - DNA, 21 bp, synthetic construct
  misc_feature 1..21: note = SEQ ID NO: 140;
  source 1..21: mol_type = other DNA
ttcaaagaagtgggaggcttg

SEQ ID NO: 141 - DNA, 22 bp, synthetic construct
  misc_feature 1..22: note = SEQ ID NO: 141;
  source 1..22: mol_type = other DNA
caagcctcccactactttgaaa

SEQ ID NO: 142 - DNA, 22 bp, synthetic construct
  misc_feature 1..22: note = SEQ ID NO: 142;
  source 1..22: mol_type = other DNA
caagcctcccactactttgaaa

SEQ ID NO: 143 - DNA, 22 bp, synthetic construct
  misc_feature 1..22: note = SEQ ID NO: 143;
  source 1..22: mol_type = other DNA
caagcctcccactactttgaaa

SEQ ID NO: 144 - DNA, 21 bp, synthetic construct
  misc_feature 1..21: note = SEQ ID NO: 144;
  source 1..21: mol_type = other DNA
ttcaaagaagtgggaggcttg

SEQ ID NO: 145 - DNA, 21 bp, synthetic construct
  misc_feature 1..21: note = SEQ ID NO: 145;
  source 1..21: mol_type = other DNA
ttcaaagaagtgggaggcttg

SEQ ID NO: 146 - DNA, 21 bp, synthetic construct
  misc_feature 1..21: note = SEQ ID NO: 146;
  source 1..21: mol_type = other DNA
ttcaaagaagtgggaggcttg

SEQ ID NO: 147 - DNA, 12 bp, synthetic construct
  misc_feature 1..12: note = SEQ ID NO: 147;
  source 1..12: mol_type = other DNA
tcccactacttt

SEQ ID NO: 148 - DNA, 12 bp, synthetic construct
  misc_feature 1..12: note = SEQ ID NO: 148;
  source 1..12: mol_type = other DNA
tcccactacttt

SEQ ID NO: 149 - DNA, 12 bp, synthetic construct
  misc_feature 1..12: note = SEQ ID NO: 149;
  source 1..12: mol_type = other DNA
tcccacttcttt

SEQ ID NO: 150 - DNA, 12 bp, synthetic construct
  misc_feature 1..12: note = SEQ ID NO: 150;
  source 1..12: mol_type = other DNA
tcccactacttt

SEQ ID NO: 151 - DNA, 20 bp, synthetic construct
  misc_feature 1..20: note = SEQ ID NO: 151;
  source 1..20: mol_type = other DNA
gcagagtggtgagtggtcag

SEQ ID NO: 152 - DNA, 21 bp, synthetic construct
  misc_feature 1..21: note = SEQ ID NO: 152;
  source 1..21: mol_type = other DNA
tctgaatctttccctggtgtc

SEQ ID NO: 153 - DNA, 20 bp, synthetic construct
  misc_feature 1..20: note = SEQ ID NO: 153;
  source 1..20: mol_type = other DNA
gtaggggaggtggtagcatg

SEQ ID NO: 154 - DNA, 20 bp, synthetic construct
  misc_feature 1..20: note = SEQ ID NO: 154;
  source 1..20: mol_type = other DNA
aaagggacaggacatggacc

SEQ ID NO: 155 - DNA, 20 bp, synthetic construct
  misc_feature 1..20: note = SEQ ID NO: 155;
  source 1..20: mol_type = other DNA
tttgtatccccaccccctta

SEQ ID NO: 156 - DNA, 24 bp, synthetic construct
  misc_feature 1..24: note = SEQ ID NO: 156;
  source 1..24: mol_type = other DNA
atgagctgctattagtcccatctg

SEQ ID NO: 157 - DNA, 20 bp, synthetic construct
  misc_feature 1..20: note = SEQ ID NO: 157;
  source 1..20: mol_type = other DNA
cagtcagtctttgggggttt

SEQ ID NO: 158 - DNA, 21 bp, synthetic construct
  misc_feature 1..21: note = SEQ ID NO: 158;
  source 1..21: mol_type = other DNA
ctggtgagtttgggattcttg

SEQ ID NO: 159 - DNA, 24 bp, synthetic construct
  misc_feature 1..24: note = SEQ ID NO: 159;
  source 1..24: mol_type = other DNA
tcttgttcaagagttccctatcac

SEQ ID NO: 160 - DNA, 21 bp, synthetic construct
  misc_feature 1..21: note = SEQ ID NO: 160;
  source 1..21: mol_type = other DNA
tgaagtaggtgcactgtttgt

SEQ ID NO: 161 - DNA, 26 bp, synthetic construct
  misc_feature 1..26: note = SEQ ID NO: 161;
  source 1..26: mol_type = other DNA
ccagaattaacagtataaattgcatc

SEQ ID NO: 162 - DNA, 23 bp, synthetic construct
  misc_feature 1..23: note = SEQ ID NO: 162;
  source 1..23: mol_type = other DNA
tgtagctgtgttttctttgtaga

SEQ ID NO: 163 - DNA, 26 bp, synthetic construct
  misc_feature 1..26: note = SEQ ID NO: 163;
  source 1..26: mol_type = other DNA
caggtaaagtctttgaaaatatgtgt

SEQ ID NO: 164 - DNA, 26 bp, synthetic construct
  misc_feature 1..26: note = SEQ ID NO: 164;
  source 1..26: mol_type = other DNA
tccattcaaaatcatctgtaaatcca

SEQ ID NO: 165 - DNA, 23 bp, synthetic construct
  misc_feature 1..23: note = SEQ ID NO: 165;
  source 1..23: mol_type = other DNA
gttaattgcatgaattagagcta

SEQ ID NO: 166 - DNA, 24 bp, synthetic construct
  misc_feature 1..24: note = SEQ ID NO: 166;
  source 1..24: mol_type = other DNA
aaaatattgtacttaccttcttgg

SEQ ID NO: 167 - DNA, 23 bp, synthetic construct
  misc_feature 1..23: note = SEQ ID NO: 167;
  source 1..23: mol_type = other DNA
ctgtttactcttgctcttgtcca

SEQ ID NO: 168 - DNA, 20 bp, synthetic construct
  misc_feature 1..20: note = SEQ ID NO: 168;
  source 1..20: mol_type = other DNA
ggcagacagggaggatgtag

SEQ ID NO: 169 - DNA, 20 bp, synthetic construct
  misc_feature 1..20: note = SEQ ID NO: 169;
  source 1..20: mol_type = other DNA
cactcccactgtcctttcct

SEQ ID NO: 170 - DNA, 23 bp, synthetic construct
  misc_feature 1..23: note = SEQ ID NO: 170;
  source 1..23: mol_type = other DNA
aggcttcattatcaaacttgggt

SEQ ID NO: 171 - DNA, 3143 bp, synthetic construct
  misc_feature 1..3143: note = SEQ ID NO: 171;
  source 1..3143: mol_type = other DNA
tttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgcaacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgccaccatggccttaccagtggccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacatgatgcaactcctgtcttgcattgcactaagtcttgcacttgtcacaaacagtgcacctacttcaagttctacaaagaaaacacagctacaactggagcatttactgctggatttacagatgattttgaatggaattaatgtaagtatatttcctttcttactaaaattattacatttagtaatctagctggagatcatttcttaataacaatgcattatactttcttagaattacaagaatcccaaactcaccaggatgctcacatttaagttttacatgcccaagaaggtaagtacaatattttatgttcaatttctgttttaataaaattcaaagtaatatgaaaatttgcacagatgggactaatagcagctcat

SEQ ID NO: 172 - protein, 485 aa, synthetic construct; identical to SEQ ID NO: 32
  REGION 1..485: note = SEQ ID NO: 172;

SEQ ID NO: 173 - protein, 20 aa, synthetic construct
  REGION 1..20: note = SEQ ID NO: 173;
GAGTTGCATCCTGTACATTG

SEQ ID NO: 174 - protein, 20 aa, synthetic construct
  REGION 1..20: note = SEQ ID NO: 174;
CAACTCCTGCCACAATGTAC

SEQ ID NO: 175 - protein, 5 aa, synthetic construct
  REGION 1..5: note = SEQ ID NO: 175;
GSGGS

SEQ ID NO: 176 - protein, 4 aa, synthetic construct
  REGION 1..4: note = SEQ ID NO: 176;
GGGS

SEQ ID NO: 177 - protein, 5 aa, synthetic construct
  REGION 1..5: note = SEQ ID NO: 177;
GGGGS

SEQ ID NO: 178 - protein, 4 aa, synthetic construct
  REGION 1..4: note = SEQ ID NO: 178;
GGSG

SEQ ID NO: 179 - protein, 5 aa, synthetic construct
  REGION 1..5: note = SEQ ID NO: 179;
GGSGG

SEQ ID NO: 180 - protein, 5 aa, synthetic construct
  REGION 1..5: note = SEQ ID NO: 180;
GSGSG

SEQ ID NO: 181 - protein, 5 aa, synthetic construct
  REGION 1..5: note = SEQ ID NO: 181;
GSGGG

SEQ ID NO: 182 - protein, 5 aa, synthetic construct
  REGION 1..5: note = SEQ ID NO: 182;
GGGSG

SEQ ID NO: 183 - protein, 5 aa, synthetic construct
  REGION 1..5: note = SEQ ID NO: 183;
GSSSG

SEQ ID NO: 184 - protein, 5 aa, synthetic construct
  REGION 1..5: note = SEQ ID NO: 184;
GGGGS

SEQ ID NO: 185 - protein, 20 aa, synthetic construct
  REGION 1..20: note = SEQ ID NO: 185;
GGGGSGGGGSGGGGSGGGGS

SEQ ID NO: 186 - protein, 5 aa, synthetic construct
  REGION 1..5: note = SEQ ID NO: 186;
GGGGS

SEQ ID NO: 187 - protein, 4 aa, synthetic construct
  REGION 1..4: note = SEQ ID NO: 187;
GGSG

SEQ ID NO: 188 - protein, 5 aa, synthetic construct
  REGION 1..5: note = SEQ ID NO: 188;
GGSGG

SEQ ID NO: 189 - protein, 5 aa, synthetic construct
  REGION 1..5: note = SEQ ID NO: 189;
GSGSG

SEQ ID NO: 190 - protein, 5 aa, synthetic construct
  REGION 1..5: note = SEQ ID NO: 190;
GSGGG

SEQ ID NO: 191 - protein, 5 aa, synthetic construct
  REGION 1..5: note = SEQ ID NO: 191;
GGGSG

SEQ ID NO: 192 - protein, 5 aa, synthetic construct
  REGION 1..5: note = SEQ ID NO: 192;
GSSSG

SEQ ID NO: 193 - protein, 5 aa, synthetic construct
  REGION 1..5: note = SEQ ID NO: 193;
GGGGS

SEQ ID NO: 194 - protein, 5 aa, synthetic construct
  REGION 1..5: note = SEQ ID NO: 194;
DKTHT

SEQ ID NO: 195 - protein, 4 aa, synthetic construct
  REGION 1..4: note = SEQ ID NO: 195;
CPPC

SEQ ID NO: 196 - protein, 15 aa, synthetic construct
  REGION 1..15: note = SEQ ID NO: 196;
CPEPKSCDTPPPCPR

SEQ ID NO: 197 - protein, 12 aa, synthetic construct
  REGION 1..12: note = SEQ ID NO: 197;
ELKTPLGDTTHT

SEQ ID NO: 198 - protein, 10 aa, synthetic construct
  REGION 1..10: note = SEQ ID NO: 198;
KSCDKTHTCP

SEQ ID NO: 199 - protein, 7 aa, synthetic construct
  REGION 1..7: note = SEQ ID NO: 199;
KCCVDCP

SEQ ID NO: 200 - protein, 7 aa, synthetic construct
  REGION 1..7: note = SEQ ID NO: 200;
KYGPPCP

SEQ ID NO: 201 - protein, 15 aa, synthetic construct
  REGION 1..15: note = SEQ ID NO: 201;
EPKSCDKTHTCPPCP

SEQ ID NO: 202 - protein, 12 aa, synthetic construct
  REGION 1..12: note = SEQ ID NO: 202;
ERKCCVECPPCP

SEQ ID NO: 203 - protein, 17 aa, synthetic construct
  REGION 1..17: note = SEQ ID NO: 203;
ELKTPLGDTTHTCPRCP

SEQ ID NO: 204 - protein, 12 aa, synthetic construct
  REGION 1..12: note = SEQ ID NO: 204;
SPNMVPHAHHAQ

SEQ ID NO: 205 - protein, 15 aa, synthetic construct
  REGION 1..15: note = SEQ ID NO: 205;
EPKSCDKTYTCPPCP

(19) United States
(12) Patent Application Publication
(10) Pub. No.: US 2023/0364238 A1
(43) Pub. Date: Nov. 16, 2023

(54) Methods and Compositions Comprising Orthogonal Cytokine Responsive Immune Cells

(71) Applicants: THE TRUSTEES OF THE UNIVERSITY OF PENNSYLVANIA, Philadelphia, PA (US); THE BOARD OF TRUSTEES OF THE LELAND STANFORD JUNIOR UNIVERSITY, Stanford, CA (US)

(72) Inventors: Michael C. Milone, Cherry Hill, NJ (US); Qian Zhang, Philadelphia, PA (US); Kenan Christopher Garcia, Menlo Park, CA (US)

(21) Appl. No.: 18/316,670

(22) Filed: May 12, 2023

Related U.S. Application Data
(60) Provisional application No. 63/341,277, filed on May 12, 2022.

(51) Int. Cl. A61K 39/00; C12N 9/22; C12N 15/11; C12N 15/90; A61P 35/00; A61K 38/20

(52) U.S. Cl. CPC A61K 39/4632; C12N 9/22; C12N 15/111; C12N 15/907; A61K 39/4611; A61K 39/4631; A61K 39/464494; A61K 39/464492; A61K 39/464435; A61K 39/464486; A61K 39/464488; A61K 39/464482; A61K 39/46447; A61K 39/464468; A61K 39/464453; A61K 39/464424; A61P 35/00; A61K 38/2013; C12N 2310/20; C12N 2800/80; A61K 2239/13; A61K 2239/22

(57) ABSTRACT

The present disclosure provides methods of producing a modified immune cell responsive to orthogonal cytokine signaling and a modified immune cell produced by said method. The present disclosure further provides a modified immune cell responsive to orthogonal cytokine signaling and methods for treating cancer comprising the modified immune cell.

### CROSS-REFERENCE TO RELATED APPLICATIONS

This application claims priority under 35 U.S.C. § 119(e) to U.S. Provisional Patent Application No. 63/341,277, filed May 12, 2022, which is incorporated herein by reference in its entirety.

### STATEMENT REGARDING FEDERALLY SPONSORED RESEARCH OR DEVELOPMENT

This invention was made with Government support under CA244711 awarded by the National Institutes of Health. The Government has certain rights in the invention.

### INCORPORATION-BY-REFERENCE OF SEQUENCE LISTING

The present application contains a Sequence Listing which has been submitted in XML format via Patent Center and is hereby incorporated by reference in its entirety. Said XML file, created on May 11, 2023, is named 046483-7370US1 Sequence Listing.xml and is 283.5 kilobytes in size.

### BACKGROUND OF THE INVENTION

The ability of tumor infiltrating lymphocytes (TILs) to induce complete regression of advanced cancers, most notably melanoma, provided some of the earliest evidence of the power of T cell-based immunotherapy. More recently, the clinical activity of two adoptive cell therapies (ACTs) based upon T cells genetically engineered to express a chimeric antigen receptor (CAR) against CD19 (namely, tisagenlecleucel (CTL019) and axicabtagene ciloleucel (KTE-C19)), illustrates the therapeutic potential of ACTs. At present, more than 350 clinical trials of T-cell based immunotherapies for both hematologic malignancies and solid tumors are underway globally, attempting to leverage engineered T cell technology.

The importance of CAR T cell expansion and persistence to the clinical efficacy of CAR T cell therapy has been well supported by correlative studies of CD19-specific CART cell kinetics following adoptive transfer. Clinical response to this therapy is highly correlated with the peak CAR T cell concentration in blood as well as the area under the concentration-time curve (AUC) for the first 28 days following infusion. Consistent with this kinetics, early loss of CTL019 persistence is also associated with relapse.

Conditioning chemotherapy enhances T cell engraftment and ACT efficacy, as initially recognized in the context of tumor infiltrating lymphocyte (TIL) therapy for melanoma where expanded TILs had minimal antitumor effect in the absence of prior conditioning chemotherapy. Despite the success, the conditioning chemotherapy, commonly cyclophosphamide and fludarabine, is not without toxicity. Both agents are associated with myelosuppression leading to neutropenia that, along with lymphopenia, increases the risk for infection. Nausea, vomiting and diarrhea are also common. Cyclophosphamide can also cause hemorrhagic cystitis requiring prophylaxis. Fludarabine also has well-described neurologic toxicity that may influence the neurotoxicity associated with CAR T cell therapy.

IL-2 plays important roles in regulating immune responses that are relevant to T cell immunotherapies. IL-2 through the IL2Rβ/γ_{c} dimer potently activates STAT5a/b, promoting T cell proliferation and the acquisition of effector molecules such as perforin in CD8+ T cells through STAT-mediated regulation of the transcription factor, Eomes. Although IL-2 potentiates effector T cells, IL-2 also supports the survival and function of regulatory T cells (Tregs), which constitutively express CD25, the high affinity receptor for IL-2. This important role of IL-2 in immunoregulation is illustrated by the paradoxical autoimmunity that arises in mice deficient in IL-2. Many tumors exhibit an expansion of Tregs within the tumor microenvironment, and they are postulated to represent a major immune checkpoint limiting adaptive immunity to cancer.

While effective at enhancing tumor immunity in melanoma and renal cell carcinoma, rhIL-2 induces significant and sometimes serious adverse effects including hypotension and renal dysfunction, a “cytokine storm” that resembles sepsis and a characteristic pulmonary vascular leak syndrome. Similar adverse effects have been observed in the first-in-human clinical trial of rhIL-15. In both cases, toxicity was dose dependent and dose limiting.

Thus, alternative approaches are needed to enhance the survival, proliferation and function of immune cells (e.g., T cells) following adoptive transfer. Such approaches would eliminate the use of toxic chemotherapeutic agents, would overcome the toxicity of rhIL-2, and would overcome the immunosuppression of IL-2 activated Tregs. The present invention addresses this need.

### SUMMARY OF THE INVENTION

In one aspect, the invention provides a method of producing a modified immune cell responsive to orthogonal cytokine signaling, the method comprising:
(a) genetically engineering an immune effector cell responsive to interleukin-2 (IL-2) and interleukin-15 (IL-15) to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell; and
(b) genetically engineering the immune effector cell to express an orthogonal IL-2 receptor beta (oIL2Rβ);
wherein step (a) and step (b) are performed in any order.

In some embodiments, step (b) comprises genetically engineering endogenous IL-2 receptor beta (IL2Rβ) gene of the immune effector cell to express the oIL2Rβ such that the modified immune cell is an endogenous IL2Rβ−/− immune cell and an oIL2Rβ+/+ immune cell.

In some embodiments, step (a) comprises a clustered regularly interspaced short palindromic repeats (CRISPR) associated nuclease (Cas nuclease) and a single-guide RNA (sgRNA) that targets the Cas nuclease to the locus within the endogenous IL-2 gene of the immune cell.

In some embodiments, the Cas nuclease is a Cas9 nuclease.

In some embodiments, step (a) comprises CRISPR/Cas-mediated homology directed repair (HDR).

In some embodiments, the genetic engineering of step (b) comprises prime editing.

In some embodiments, the prime editing comprises a Cas9 nickase-reverse transcriptase and a prime editing guide RNA (pegRNA).

In some embodiments, the immune cell is a human immune cell, the prime editing comprises introducing a first point mutation and a second point mutation into the endogenous IL2Rβ gene, and the first point mutation results in a H133D amino acid change and and the second point mutation results in a Y134F amino acid change.

In some embodiments, the first point mutation is C397G and the second point mutation is A401T.

In some embodiments, the immune cell is a human immune cell, and the oIL2Rβ comprises H133D and Y134F mutations relative to endogenous IL2Rβ.

In some embodiments, the modified immune cell is responsive to an orthogonal IL-2 (oIL2).

In some embodiments, the oIL2 binds to the oIL2Rβ.

In some embodiments, the immune cell is a T cell.

In some embodiments, the immune cell is a human T cell.

In some embodiments, step (a) comprises genetically engineering the immune cell to express a TCR, and the TCR targets a tumor antigen; or step (a) comprises genetically engineering the immune cell to express a CAR, and the CAR targets a tumor antigen.

In some embodiments, the tumor antigen is selected from the group consisting of CD19, CD20, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, gp100, Glycolipid F77, FAP, EGFRvIII, MAGE A3, 5T4, WT1, KG2D ligand, folate receptor alpha (FRa), and a Wnt1 antigen.

In some embodiments, the CAR comprises an extracellular antigen binding domain, a transmembrane domain, and an intracellular domain.

In some embodiments, the antigen binding domain is selected from the group consisting of a full-length antibody or antigen-binding fragment thereof, a Fab, a single-chain variable fragment (scFv), or a single-domain antibody.

In some embodiments, the antigen binding domain is an scFv.

In some embodiments, the antigen binding domain is an anti-CD19 scFv.

In some embodiments, the intracellular domain of the CAR comprises:
a costimulatory domain, or a variant thereof, of a protein selected from the group consisting of a protein in the TNFR superfamily, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS, NKG2C, B7-H3 (CD276), and any combination thereof; or
an intracellular domain derived from a killer immunoglobulin-like receptor (KIR).

In some embodiments, the intracellular domain of the CAR comprises or further comprises an intracellular signaling domain, or a variant thereof, of a protein selected from the group consisting of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d.

In some embodiments, the CAR comprises an anti-CD19 scFv, a transmembrane domain, and an intracellular domain comprising a 4-1BB costimulatory domain and a CD3 zeta signaling domain.

In one aspect, the invention provides a modified immune cell responsive to orthogonal cytokine signaling, wherein the modified immune cell is derived from an immune effector cell responsive to interleukin-2 (IL-2) and interleukin-15 (IL-15); and wherein the modified immune cell:
(a) expresses a T cell receptor (TCR) or a chimeric antigen receptor (CAR) from an exogenous nucleic acid inserted at a locus within endogenous IL-2 gene of the immune cell, wherein the exogenous nucleic acid comprises a polynucleotide sequence encoding the TCR or the CAR, such that the modified immune cell is an IL2−/− immune cell; and
(b) expresses an orthogonal IL-2 receptor beta (oIL2Rβ).

In some embodiments, the modified immune cell is an endogenous IL2Rβ−/− immune cell.

In some embodiments, the endogenous IL2Rβ gene is edited such that it encodes the oIL2Rβ.

In some embodiments, the immune effector cell is a human immune cell, the edited endogenous IL2Rβ gene comprises a first point mutation and a second point mutation, and the first point mutation results in a H133D amino acid change and and the second point mutation results in a Y134F amino acid change relative to endogenous IL2Rβ.

In some embodiments, the first point mutation is C397G and the second point mutation is A401T.

In some embodiments, the immune effector cell is a human immune cell, and the oIL2Rβ comprises H133D and Y134F mutations relative to IL2Rβ.

In some embodiments, the modified immune cell is responsive to an orthogonal IL-2 (oIL2).

In some embodiments, the oIL2 binds to the oIL2Rβ.

In some embodiments, the immune effector cell is a T cell.

In some embodiments, the immune effector cell is a human T cell.

In some embodiments, the TCR targets a tumor antigen, or the CAR targets a tumor antigen.

In some embodiments, the tumor antigen is selected from the group consisting of CD19, CD20, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, gp100, Glycolipid F77, FAP, EGFRvIII, MAGE A3, 5T4, WT1, KG2D ligand, folate receptor alpha (FRa), and a Wnt1 antigen.

In some embodiments, the CAR comprises an extracellular antigen binding domain, a transmembrane domain, and an intracellular domain.

In some embodiments, the antigen binding domain is selected from the group consisting of a full-length antibody or antigen-binding fragment thereof, a Fab, a single-chain variable fragment (scFv), or a single-domain antibody.

In some embodiments, the antigen binding domain is an scFv.

In some embodiments, the antigen binding domain is an anti-CD19 scFv.

In some embodiments, the intracellular domain of the CAR comprises:
a costimulatory domain, or a functional variant thereof, of a protein selected from the group consisting of a protein in the TNFR superfamily, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS, NKG2C, B7-H3 (CD276), and any combination thereof; or an intracellular domain derived from a killer immunoglobulin-like receptor (KIR).

In some embodiments, the intracellular domain of the CAR comprises or further comprises an intracellular signaling domain, or a functional variant thereof, of a protein selected from the group consisting of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d.

In some embodiments, the CAR comprises an anti-CD19 scFv, a transmembrane domain, and an intracellular domain comprising a 4-1BB costimulatory domain and a CD3 zeta signaling domain.

In some embodiments, the modified immune cell is produced by a method disclosed herein.

In one aspect, the invention provides a method of treating cancer in a subject, the method comprising:
(a) administering to the subject an effective amount of the modified immune cell responsive to orthogonal cytokine signaling of any one of embodiments 23-41; and
(b) administering to the subject an effective amount of an orthogonal interleukin-2 (oIL2) which binds to the oIL2Rβ, or a vector which expresses the oIL2.

In some embodiments, the vector which expresses oIL2 is a viral vector.

In some embodiments, the viral vector is selected from an adenoviral vector, an adeno-associated virus (AAV) vector, a lentiviral vector, and a retroviral vector.

In some embodiments, the administering comprises intravenous administration and/or intratumoral injection.

In some embodiments, the immune effector cell is a human cell and the subject is a human.

In some embodiments, the immune effector cell is a human T cell and the subject is a human.

In some embodiments, the method further comprises discontinuing administration of the oIL2 or the vector which expresses the oIL2.

In one aspect, the invention provides a method of producing a modified immune cell responsive to orthogonal cytokine signaling, the method comprising:
(a) genetically engineering an immune effector cell responsive to interleukin-2 (IL-2) and interleukin-15 (IL-15) to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell; and
(b) genetically engineering endogenous IL-2 receptor beta (IL2Rβ) gene of the immune effector cell to express the oIL2Rβ such that the modified immune cell is an endogenous IL2Rβ−/− immune cell and an oIL2Rβ+/+ immune cell;
wherein step (a) and step (b) are performed in any order; and

further wherein step (a) comprises CRISPR/Cas-mediated homology directed repair (HDR) and step (b) comprises prime editing.

In one aspect, the invention provides a modified immune cell responsive to orthogonal cytokine signaling, wherein the modified immune cell is derived from an immune effector cell responsive to interleukin-2 (IL-2) and interleukin-15 (IL-15); and wherein the modified immune cell:
(a) expresses a T cell receptor (TCR) or a chimeric antigen receptor (CAR) from an exogenous nucleic acid inserted at a locus within endogenous IL-2 gene of the immune cell, wherein the exogenous nucleic acid comprises a polynucleotide sequence encoding the TCR or the CAR, such that the modified immune cell is an IL2−/− immune cell; and
(b) expresses an orthogonal IL-2 receptor beta (oIL2Rβ);
wherein the endogenous IL2Rβ gene is edited such that it encodes the oIL2Rβ.

In one aspect, the invention provides a method of producing a modified immune cell responsive to orthogonal cytokine signaling, the method comprising genetically engineering at least one endogenous IL-2 receptor beta (IL2Rβ) gene of the immune effector cell to express an orthogonal IL-2 receptor beta (oIL2Rβ), wherein the modified immune cell is derived from an immune effector cell responsive to interleukin-2 (IL-2) and interleukin-15 (IL-15); further wherein the genetic engineering comprises prime editing, and wherein the prime editing comprises a prime editing guide RNA (pegRNA) comprising or consisting of SEQ ID NO: 1.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 is a table showing pegRNAs (SEQ ID NOs: 1, 6, 11, 16, and 21) used for prime editing of endogenous IL-2 receptor beta (IL2Rβ). The sgRNA regions (SEQ ID NOs: 2, 7, 12, 17, and 22), the scaffold regions (SEQ ID NOs: 3, 8, 13, 18, and 23), and the PBS regions (SEQ ID NOs: 5, 10, 15, 20, and 25) are highlighted. The RT template regions (SEQ ID NOs: 4, 9, 14, 19, and 24) are underlined.

FIG. 2 is a schematic illustrating the orthogonal IL-2 (oIL2)/IL-2 receptor (oIL2R) system used in the present study. Informed by a structural understanding of the interactions between IL-2 and IL-2Rβ, targeted mutations within the cytokine-receptor interface are introduced to generate mutated IL-2 (“orthoIL2” or “oIL2”) and mutated IL2Rβ chain (“orthoIL2Rβ” or CoIL2Rβ″) with binding that is orthogonal to natural endogenous IL-2 and IL2Rβ.

FIG. 3 is a schematic of the process used herein for producing an orthoIL2Rb+IL2−/− CAR T cell through gene editing.

FIG. 4 illustrates the results that prime editing is able to introduce two mutations in IL2Rb necessary to confer IL-2 orthogonality in human CAR T cells. SEQ ID NO: 108 and SEQ ID NO: 109 are shown.

FIG. 5 illustrates the results that CRISPR/Cas9 mediated HDR is able to introduce a CD19-specific CAR into the IL-2 locus. CAR expression in this design is driven by the endogenous IL-2 promoter. SEQ ID NO: 110 and SEQ ID NO: 111 are shown.

FIG. 6 illustrates the results that orthoIL2Rb+ CAR T cells generated by CRISPR/Cas9 mediated HDR (CAR19) show cytolytic activity that varies with E:T ratio when compared to mock or non-transduced T cells (NTD).

FIGS. 7A-7D relate to the evaluation of hIL2 sgRNA:Cas9-mediated mutagenesis of human IL2. FIG. 7A is a schematic diagram of sgRNA targeting at hIL2 Exon 1 locus. SEQ ID NO: 112 and SEQ ID NO: 113 are shown. The sgRNA targeting site (SEQ ID NO: 114) on the antisense strand is highlighted, the protospacer adjacent motif (PAM) sequence (CCA) is labeled, and the expected cleavage site within the translation initiation codon (ATG) is indicated by the vertical arrowhead. FIG. 7B shows detection of sgRNA:Cas9-mediated cleavage of hIL2 from cells by western blot analysis. FIG. 7C is a chart showing detection of circulating IL2 from supernatant via Elisa assay. FIG. 7D is an illustration of the data output from the ICE (Inference of CRISPR Edits) software for the guide targeting the human IL2 gene. The edited and control samples were sequence ed with Sanger sequencing (SEQ ID NO: 115 and SEQ ID NO: 116) and further analyzed with ICE. First panel: trace file segments spanning the cut site from the control and the edited sample are generated for every analysis. The guide target sequence (SEQ ID NO: 117) is underlined in black, and the PAM sequence (CCA) is denoted by a dotted red underline in the control sample. Vertical dotted lines denote the expected cut site. Second panel: discordance for the edited (green) and control (orange) trace files. The vertical dotted line marks the cut site. The alignment window marks the region of the traces with high Phred scores that is used to align the edited and control traces. The inference window marks the region of the traces around the cut site, which will be used to infer the change in sequence between the edited and control traces. Third panel: as calculated by ICE, insert, or deletion (indel) sizes and their relative prevalence for IL2 gene. Fourth panel: exact sequence calls and their relative prevalence (SEQ ID NOs: 118-132). Vertical dotted lines denote the targeted cut sites.

FIGS. 8A-8D relate to CRISPR/Cas9-targeted CAR19 gene integration into the IL2 locus with promoter-containing donor plasmid DNAs. FIG. 8A is a schematic representation of design to edit CAR into the human IL2 locus. Top, IL2 locus; middle, donor DNA containing a promoter and the 19BBz CAR flanked by homology arms; bottom, edited CAR locus. FIG. 8B is a schematic representation of Cas9:single-guide RNA ribonucleoprotein (Cas9 RNP) delivery to primary human T cells for genome editing, followed by genetic and phenotypic characterization. FIG. 8C is a series of representative CAR FACS flow plots 4 days after IL2 targeting. FACS plots show increasing percentages of CAR19 with higher concentrations of donor DNA compared with control-treated cells (Cas9 without sgRNA). FIG. 8D relates to validation of CRISPR/Cas9-mediated knock-in of CAR19 at IL2 locus. First panel shows a schematic indicating the position of two-pair primers flanking the knock-in sites and an agarose gel showing PCR amplification of knock-in region using the two-pairs primers. Second panel shows DNA sequencing analysis of the amplified DNA fragments, which revealed that the CAR donor DNA was correctly knocked-in at the IL2 gene locus. SEQ ID NO: 133 and SEQ ID NO: 134 are shown.

FIGS. 9A-9C relate to CAR19-engineered T cells recognize and kill antigen-expressing target cells. FIG. 9A shows evaluation of edited CAR-T cell cytotoxicity using image-based Agilent eSight assay. Killing of GFP-expressing K562-CD19 cells by edited CD19 CAR-T cells with indicated doses of donor DNA concentration at a specific E:T ratio (5:1). Untreated target cells and target cells treated with 0.1% Triton X-100 (100% lysis control) are used as control. FIG. 9B shows time-dependent fluorescent images for GFP+K562-CD19 cells treated with edited CAR-T, as well as the unedited T cells. FIG. 9C shows two bar graphs showing IFN-γ and TNF-α production, respectively, by CAR19 knock-in T cells stimulated with indicated concentrations of donor DNA. Bars represent median values with range (n=3).

FIGS. 10A-10G relate to prime editing to generate the human orthogonal IL2 receptor. FIG. 10A is a schematic diagram of prime editing using PE3 strategy which utilizes a pegRNA matching the target locus and a separate sgRNA that targets upstream of the edit site. The full-length pegRNA sequence (SEQ ID NO: 26) is shown including sgRNA in blue (SEQ ID NO: 27), scaffold in underlined (SEQ ID NO: 28), PBS in yellow (SEQ ID NO: 30), and RT in green (SEQ ID NO: 29) with edit sites (red) and PE3 nicking sgRNA sequence (SEQ ID NO: 31). FIG. 10B is a diagram showing optimization strategy of five pegRNAs targeting wt-IL2Rb exon 1 with various RT and PBS. FIG. 10C is a chart showing prime editing efficiency by Next-Gen Sequencing (NGS). FIG. 10D shows sanger sequencing chromatograms of the PCR fragments from control and prime-edited cells with five pegRNAs using IL2-dependant SeAx cells. Double peaks represent heterozygous or chimeric mutations, and circle indicates mutations induced by PE3. SEQ ID NOs: 135-146 are shown. FIG. 10E is a graph showing that oIL-2 expands oIL2Rβ edited human primary T cells (as well as oIL2Rβ edited SeAx cells (data not shown)). FIG. 10F shows that oIL2 induces the main signal pathways including phosphorylation of STAT5 and ERK through the edited oIL2Rβ with human primary T cells. FIG. 10G is sequencing data showing that oIL2 selectively expands the oIL2Rβ edited T cells. The oIL2Rb edited T cells increased in oIL2 culture and decreased in wt-IL2 culture. SEQ ID NOs: 147-150 are shown.

FIGS. 11A-11F relate to engineered orthoCAR19 T cells show anti-leukemic activity. FIG. 11A is a schematic of the experimental timeline for the in vivo evaluation of edited orthoCAR T cells anti-leukemic activity with ortho-hIL-2 support. NSG mice were engrafted with 1e6 CBG-labeled CD19+ Nalm6 leukemic cells on day 0. Mice received 1e6 CAR T cells (transduced, edited, and transduced with edited) were injected on day 5 following BLI on day 4. Tumor burden was assessed via bioluminescent imaging twice per week and CAR T cell expansion was examined weekly for 3 to 4 weeks. FIG. 11B is a graph of mouse body weight over time normalized to the body weight on day 0 for each mouse receiving PBS (solid lines) or 20K or 40K IU of oIL2 (dashed lines) once a day. Top: Transduced CAR with prime edited orthoIL2Rb with (dashed line) or without (solid line) 40K units of orthoIL2. Middle: Transduced CAR with transduced orthoIL2Rb with (dashed line) or without (solid line) 20K units of orthoIL2. Bottom: Overlay of the top and middle data. FIG. 11C is a graph of mouse body weight over time normalized to the body weight on day 0 for each mouse receiving PBS (solid lines) or 20K or 40K IU of oIL2 (dashed lines) once a day. Top: CAR knock-in at IL2 locus with prime edited orthoIL2Rb with (dashed line) or without (solid line) 40K units of orthoIL2. Middle: Transduced CAR with transduced orthoIL2Rb with (dashed line) or without (solid line) 20K units of orthoIL2. Bottom: Overlay of the top and middle data. FIG. 11D is a graph of individual and average BLI intensity of Nalm6-LUC determined for mice infused with T cells having CAR knock-in at IL2 locus with prime edited orthoIL2Rb with (dashed line) or without (solid line) 40K units of orthoIL2. FIG. 11E is a graph of individual and average BLI intensity of Nalm6-LUC determined for mice infused with T cells having transduced CAR with prime edited orthoIL2Rb with (dashed line) or without (solid line) 40K units of orthoIL2. FIG. 11F is a graph of individual and average BLI intensity of Nalm6-LUC determined for mice infused with T cells having transduced CAR with transduced orthoIL2Rb with (dashed line) or without (solid line) 20K units of orthoIL2.

FIGS. 12A-12G relate to engineered orthoCAR19 T cells show anti-leukemic activity. FIG. 12A is a schematic of the experimental timeline for the in vivo evaluation of edited orthoCAR T cells anti-leukemic activity with ortho-hIL-2 support. NSG mice were engrafted with 1e6 CBG-labeled CD19+ Nalm6 leukemic cells on day 0. Mice received 1e6 CAR T cells (transduced, edited, and control) were injected on day 5 following BLI on day 4. Tumor burden was assessed via bioluminescent imaging twice per week and CAR T cell expansion was examined weekly for 3 weeks. FIG. 12B is a graph of average BLI intensity of Nalm6-LUC for mice infused with the indicated treatment. FIG. 12C is a chart of the cell expansion data at Week 1. FIG. 12D is a chart of the cell expansion data at Week 2. FIG. 12E is a chart of the cell expansion data at Week 3. FIG. 12F is a series of representative CAR FACS flow plots showing CAR T cell expansion for edited CAR19 T cells and transduced CAR19 T cells in the presence of PBS or oIL2. FIG. 12G is a graph of mouse body weight over time normalized to the body weight on day 0 for each mouse receiving PBS or 20K IU of oIL2 as indicated once a day.

### DETAILED DESCRIPTION

In one aspect, the present invention provides modified immune cells (e.g., modified T cells) responsive to orthogonal cytokine signaling and methods of producing the modified immune cells. Also provided are methods of using the modified immune cells to treat diseases such as cancer.

It is to be understood that the methods described in this disclosure are not limited to particular methods and experimental conditions disclosed herein as such methods and conditions may vary. It is also to be understood that the terminology used herein is for the purpose of describing particular embodiments only and is not intended to be limiting.

Furthermore, the experiments described herein, unless otherwise indicated, use conventional molecular and cellular biological and immunological techniques within the skill of the art. Such techniques are well known to the skilled worker and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA or mRNA, which comprises a nucleotide sequence es or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequence es of more than one gene and that these nucleotide sequence es are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to, an MEW class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequence es of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes, i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequence es operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequence es have the same residues at the same positions, e.g., if a position in each of two polypeptide molecules is occupied by an arginine, then they are identical at that position. The identity or extent to which two amino acid sequence es have the same residues at the same positions in an alignment is often expressed as a percentage. The identity between two amino acid sequence es is a direct function of the number of matching or identical positions, e.g., if half (e.g., five positions in a polymer ten amino acids in length) of the positions in two sequence es are identical, the two sequence es are 50% identical; if 90% of the positions (e.g., 9 of 10), are matched or identical, the two amino acids sequence es are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence e” includes all nucleotide sequence es that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequence es which are obtained by any means available in the art, including, without limitation, recombinant means, i.e., the cloning of nucleic acid sequence es from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used herein, may be a human or non-human mammal. Non-human mammals include, for example, livestock and pets, such as ovine, bovine, porcine, canine, feline and murine mammals, as well as simian and non-human primate mammals. Preferably, the subject is human.

A “target site” or “target sequence e” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

An “ortholog”, or “orthogonal cytokine/receptor pair” refers to a genetically engineered pair of proteins that are modified by amino acid changes to (a) exhibit significantly reduced affinity to the native cytokine or cognate receptor; and (b) to specifically bind to the counterpart engineered (orthogonal) cytokine or receptor. Upon binding of the orthogonal cytokine, the orthogonal cytokine receptor activates signaling that is transduced through native cellular elements to provide for a biological activity that mimics that native response, but which is specific to an engineered cell expressing the orthogonal receptor. Enginereerd orthogonal cytokine/receptor pairs are described, e.g., in WO 2017/044464 and WO 2019/173773, which are incorporated herein by reference. Orthogonal cytokine/receptor pairs are used to direct the activity of a promiscuous cytokine to an immune cell subset of interest, thereby enabling precise control over immune cell function though genetic engineering.

An “orthogonal chimeric cytokine receptor” refers to a cytokine receptor comprising an extracellular domain of an orthogonal cytokine receptor and an intracellular signaling domain of a cytokine receptor which is distinct from the cytokine receptor from which the orthogonal cytokine receptor is derived (e.g., an oIL2-IL9 receptor). Upon binding of the orthogonal cytokine, the orthogonal chimeric cytokine receptor activates signaling that is transduced through native cellular elements to provide for a biological activity that mimics the native response of the receptor from which the intracellular signaling domain is derived, but which is specific to an engineered cell expressing the orthogonal chimeric cytokine receptor. Orthogonal chimeric cytokine receptors are described in WO 2021/050752, which is incorporated herein by reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Method of Producing a Modified Immune Cell Responsive to Orthogonal Cytokine Signaling

In one aspect, the present invention provides a method of producing a modified immune cell that is responsive to orthogonal cytokine signaling. The method comprises (a) genetically engineering an immune effector cell responsive to interleukin-2 (IL-2) and interleukin-15 (IL-15) to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell; and (b) genetically engineering the immune effector cell to express an orthogonal IL-2 receptor beta (oIL2Rβ); wherein step (a) and step (b) are performed in any order.

Orthogonal cytokine receptor/cytokine pairs are described, e.g., in WO 2017/044464, WO 2019/173773, and WO 2021/050752, which are each incorporated herein by reference. Upon binding the orthogonal IL2 cytokine (oIL2) by the orthogonal IL-2 cytokine receptor (oIL2R) extracellular domain, the orthogonal cytokine receptor activates signaling of the intracellular signaling domain that is transduced through native cellular elements to provide for a biological activity that mimics the native response of the receptor from which the orthogonal cytokine receptor is derived, but which is specific to the engineered cell expressing the orthogonal cytokine receptor. The orthogonal cytokine receptor does not bind to the endogenous counterpart cytokine, including the native counterpart of the orthogonal cytokine (e.g., IL-2), while the orthogonal cytokine (e.g., oIL2) does not bind to any endogenous receptors, including the native counterpart of the orthogonal receptor from which the orthogonal cytokine receptor is derived (e.g., IL2Rβ). The affinity of the orthogonal cytokine for the orthogonal cytokine receptor is comparable to the affinity of the native cytokine for the native receptor from which the orthogonal extracellular domain is derived. In certain embodiments of the present invention, the orthogonal cytokine receptor is oIL2Rβ. In certain embodiments of the present invention, the orthogonal cytokine receptor is an orthogonal chimeric cytokine receptor (e.g., an oIL2-IL9 receptor).

In certain embodiments of the method, genetically engineering the immune effector cell to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell (i.e., method step (a)) comprises a clustered regularly interspaced short palindromic repeats (CRISPR) associated nuclease (Cas nuclease) and a single-guide RNA (sgRNA) that targets the Cas nuclease to the locus within the endogenous IL-2 gene of the immune cell. A person of skill in the art will understand that numerous examples of CRISPR associated nucleases are known in the art. The invention includes any CRISPR associated nuclease which may be targeted using one or more guide RNAs to produce a double strand break at the endogenous IL-2 gene locus. In some embodiments, the Cas nuclease is a Cas9 nuclease. In some embodiments, the Cas nuclease is a Cas9 nickase mutant used together with paired guide RNAs to introduce targeted double-strand breaks. In certain embodiments, method step (a) comprises CRISPR/Cas-mediated homology directed repair (HDR) to insert a gene which encodes the TCR or a gene which encodes the CAR at the endogenous IL-2 gene locus.

In certain embodiments of the method, genetically engineering the immune effector cell to express an orthogonal IL-2 receptor beta (oIL2Rβ) (i.e., method step (b)) comprises genetically engineering an endogenous IL-2 receptor beta (IL2Rβ) gene of the immune effector cell to express the oIL2Rβ such that the modified immune cell is an endogenous IL2Rβ−/− immune cell and an oIL2Rβ+/+ immune cell. In some embodiments of the method, genetically engineering the immune effector cell to express an orthogonal IL-2 receptor beta (oIL2Rβ) (i.e., method step (b)) comprises genetically engineering an endogenous IL-2 receptor beta (IL2Rβ) gene of the immune effector cell to express the oIL2Rβ such that the modified immune cell is an endogenous IL2Rβ+/− immune cell and an oIL2Rβ−/+ immune cell. In certain embodiments, method step (b) comprises prime editing. In some embodiments, the prime editing comprises a Cas9 nickase-reverse transcriptase and a prime editing guide RNA (pegRNA). A person of skill in the art is familiar with prime editing techniques which are described, for example, in Anzalone, of al., (2019) Nature, 576(7785):149-157; Liu, et al., (2021) Nature Communications, 12(1):2121; Adikusuma; et al., (2021) Nucleic Acids Research. 49 (18): 10785-10795; Nelson; James (2021) Nature Biotechnology, 40 (432): 402-410.

In certain embodiments of the method, the immune cell is a human immune cell, and the prime editing comprises introducing a first point mutation and a second point mutation into the endogenous IL2Rβ gene, wherein the first point mutation results in a H133D amino acid change and and the second point mutation results in a Y134F amino acid change. In some embodiments, the first point mutation is C397G and the second point mutation is A401T. In some embodiments, the immune cell is a human immune cell, and the oIL2Rβ comprises H133D and Y134F mutations relative to endogenous IL2Rβ.

In certain embodiments of the method, the modified immune cell is responsive to an orthogonal IL-2 (oIL2). In some embodiments, the oIL2 binds to the oIL2Rβ.

In certain embodiments of the method, the immune effector cell is a T cell. In certain embodiments of the method, the immune effector cell is a human T cell.

In certain embodiments of the method, method step (a) comprises genetically engineering the immune cell to express a TCR, and the TCR targets a tumor antigen, or method step (a) comprises genetically engineering the immune cell to express a CAR, and the CAR targets a tumor antigen. Non-limiting examples of tumor antigens include CD19, CD20, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, gp100, Glycolipid F77, FAP, EGFRvIII, MAGE A3, 5T4, WT1, KG2D ligand, folate receptor alpha (FRa), and a Wnt1 antigen.

A person of skill in the art will understand that the method is not limited to producing a modified immune cell which expresses a specific TCR or CAR and that the immune cell may be engineered to express any TCR or CAR, such as a TCR or CAR useful in the treatment of a subject for a disease or condition such as cancer, an autoimmune disease, or a viral disease (e.g., AIDS). In certain embodiments, the method comprises engineering the immune cell to express any one of the TCRs or any one of the CARs disclosed herein.

In certain embodiments of the method, the CAR comprises an extracellular antigen binding domain, a transmembrane domain, and an intracellular domain. In some embodiments, the antigen binding domain is selected from the group consisting of a full-length antibody or antigen-binding fragment thereof, a Fab, a single-chain variable fragment (scFv), or a single-domain antibody. In some embodiments, the antigen binding domain is an scFv. In some embodiments, the antigen binding domain is an anti-CD19 scFv. In some embodiments, the CAR comprises an anti-CD19 scFv, a transmembrane domain, and an intracellular domain comprising a 4-1BB costimulatory domain and a CD3 zeta signaling domain.

In one aspect, the invention provides a method of producing a modified immune cell responsive to orthogonal cytokine signaling, the method comprising: (a) genetically engineering an immune effector cell responsive to interleukin-2 (IL-2) and interleukin-15 (IL-15) to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell; and (b) genetically engineering endogenous IL-2 receptor beta (IL2Rβ) gene of the immune effector cell to express the oIL2Rβ such that the modified immune cell is an endogenous IL2Rβ−/− immune cell and an oIL2Rβ+/+ immune cell; wherein step (a) and step (b) are performed in any order; and further wherein step (a) comprises CRISPR/Cas-mediated homology directed repair (HDR) and step (b) comprises prime editing.

In one aspect, the invention provides a method of producing a modified immune cell responsive to orthogonal cytokine signaling, the method comprising: (a) genetically engineering an immune effector cell responsive to interleukin-2 (IL-2) and interleukin-15 (IL-15) to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell; and (b) genetically engineering endogenous IL-2 receptor beta (IL2Rβ) gene of the immune effector cell to express the oIL2Rβ such that the modified immune cell is an endogenous IL2Rβ+/− immune cell and an oIL2Rβ−/+ immune cell; wherein step (a) and step (b) are performed in any order; and further wherein step (a) comprises CRISPR/Cas-mediated homology directed repair (HDR) and step (b) comprises prime editing.

In another aspect, the invention provides a method of producing a modified immune cell responsive to orthogonal cytokine signaling, the method comprising genetically engineering at least one endogenous IL-2 receptor beta (IL2Rβ) gene of the immune effector cell to express an orthogonal IL-2 receptor beta (oIL2Rβ), wherein the modified immune cell is derived from an immune effector cell responsive to interleukin-2 (IL-2) and interleukin-15 (IL-15); further wherein the genetic engineering comprises prime editing, and wherein the prime editing comprises a prime editing guide RNA (pegRNA) comprising or consisting of SEQ ID NO: 1 (CCAGGUGUCUUUCAAAGUAGGUUUUAGAGCUAGAAAUAGCAAGUUAAAAUAAGG CUAGUCCGUUAUCAACUUGAAAAAGUGGCACCGAGUCGGUGCAGCCUCCGACUUC UUUGAAAGACACCU).

### C. Modified Immune Cells Responsive to Orthogonal Cytokine Signaling

In one aspect, the present invention provides a modified immune cell responsive to orthogonal cytokine signaling. The modified immune cell is derived from an immune effector cell responsive to interleukin-2 (IL-2) and interleukin-15 (IL-15). The modified immune cell expresses a T cell receptor (TCR) or a chimeric antigen receptor (CAR) from an exogenous nucleic acid inserted at a locus within endogenous IL-2 gene of the immune cell. The exogenous nucleic acid comprises a polynucleotide sequence encoding the TCR or the CAR, such that the modified immune cell is an IL2−/− immune cell. The modified immune cell further expresses an orthogonal IL-2 receptor beta (oIL2Rβ).

In certain embodiments, the modified immune cell is an endogenous IL2Rβ−/− immune cell. In certain embodiments, the modified immune cell is an endogenous IL2Rβ+/− immune cell. In some embodiments, the endogenous IL2Rβ gene of the modified immune cell is edited such that it encodes the oIL2Rβ. In some embodiments, the immune effector cell is a human immune cell, and the edited endogenous IL2Rβ gene comprises a first point mutation and a second point mutation, wherein the first point mutation results in a H133D amino acid change and and the second point mutation results in a Y134F amino acid change relative to endogenous IL2Rβ. In some embodiments, the first point mutation is C397G and the second point mutation is A401T. In some embodiments, the immune effector cell is a human immune cell, and the oIL2Rβ comprises H133D and Y134F mutations relative to IL2Rβ.

In other embodiments, the modified immune cell is transduced with a viral vector, such as a lentiviral vector, encoding the oIL2Rβ. In some embodiments, the immune effector cell is a human immune cell, and the oIL2Rβ comprises H133D and Y134F mutations relative to IL2Rβ.

In certain embodiments, the modified immune cell is responsive to an orthogonal IL-2 (oIL2). In some embodiments, the oIL2 binds to the oIL2Rβ.

In certain embodiments, the immune effector cell is a T cell. In certain embodiments, the immune effector cell is a human T cell.

In certain embodiments, the modified immune cell expresses a TCR and the TCR targets a tumor antigen. In some embodiments, the modified immune cell expresses a CAR and the CAR targets a tumor antigen. Non-limiting examples of tumor antigens include CD19, CD20, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, gp100, Glycolipid F77, FAP, EGFRvIII, MAGE A3, 5T4, WT1, KG2D ligand, folate receptor alpha (FRa), and a Wnt1 antigen.

A person of skill in the art will understand that the invention is not limited to a modified immune cell which expresses a specific TCR or CAR and that the modified immune cell may express any TCR or CAR, such as a TCR or CAR useful in the treatment of a subject for a disease or condition such as cancer, an autoimmune disease, or a viral disease (e.g., AIDS). In certain embodiments, the modified immune cell expresses any one of the TCRs or any one of the CARs disclosed herein.

In certain embodiments of the modified immune cell, the CAR comprises an extracellular antigen binding domain, a transmembrane domain, and an intracellular domain. In some embodiments, the antigen binding domain is selected from the group consisting of a full length antibody or antigen-binding fragment thereof, a Fab, a single-chain variable fragment (scFv), or a single-domain antibody. In some embodiments, the antigen binding domain is an scFv. In some embodiments, the antigen binding domain is an anti-CD19 scFv. In some embodiments, the CAR comprises an anti-CD19 scFv, a transmembrane domain, and an intracellular domain comprising a 4-1BB costimulatory domain and a CD3 zeta signaling domain.

In one aspect, the invention provides a modified immune cell responsive to orthogonal cytokine signaling, wherein the modified immune cell is derived from an immune effector cell responsive to interleukin-2 (IL-2) and interleukin-15 (IL-15); and wherein the modified immune cell: (a) expresses a T cell receptor (TCR) or a chimeric antigen receptor (CAR) from an exogenous nucleic acid inserted at a locus within endogenous IL-2 gene of the immune cell, wherein the exogenous nucleic acid comprises a polynucleotide sequence encoding the TCR or the CAR, such that the modified immune cell is an IL2−/− immune cell; and (b) expresses an orthogonal IL-2 receptor beta (oIL2Rβ; wherein the endogenous IL2Rβ gene is edited such that it encodes the oIL2Rβ.

### D. Chimeric Antigen Receptors (CARs)

In some aspects, the invention provides a method of producing a modified immune cell (e.g., T cell) responsive to orthogonal cytokine signaling, in which the modified immune cell is an immune effector cell which has been modified to express a chimeric antigen receptor (CAR). In some aspects, the invention provides a modified immune cell (e.g., T cell) responsive to orthogonal cytokine signaling, in which the modified immune cell is an immune effector cell which has been modified to express a chimeric antigen receptor (CAR). In certain embodiments, the CAR comprises an extracellular antigen binding domain, a transmembrane domain, and an intracellular domain as described herein.

### Antigen Binding Domains

The antigen binding domain of a CAR is an extracellular region of the CAR for binding to a specific target antigen including proteins, carbohydrates, and glycolipids. The antigen binding domain can include any domain that binds one or more antigen(s) and may include, but is not limited to, a monoclonal antibody (mAb), a polyclonal antibody, a synthetic antibody, a bispecific antibody, a human antibody, a humanized antibody, a non-human antibody, a single-domain antibody, a full-length antibody or any antigen-binding fragment thereof, a Fab, and a single-chain variable fragment (scFv). In some embodiments, the antigen binding domain comprises an aglycosylated antibody or a fragment thereof or scFv thereof.

In some embodiments, the target antigen recognized by the antigen binding comprises a tumor antigen. Examples of tumor antigens that may be targeted by the antigen binding domain of the CAR include one or more antigens selected from the group including, but not limited to, the CD19, CD20, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, Glycolipid F77, FAP, EGFRvIII, MAGE A3, 5T4, WT1, KG2D ligand, a folate receptor (FRa), and Wnt1 antigens.

As used herein, the term “single-chain variable fragment” or “scFv” is a fusion protein of the variable regions of the heavy (VH) and light (VL) chains of an immunoglobulin (e.g., mouse or human) covalently linked to form a VH::VL heterodimer. The variable heavy (VH) and light (VL) chains are either joined directly or joined by a peptide linker, which connects the N-terminus of the VH with the C-terminus of the VL, or the C-terminus of the VH with the N-terminus of the VL. In some embodiments, the antigen binding domain comprises an scFv having the configuration from N-terminus to C-terminus, VH—linker—VL. In some embodiments, the antigen binding domain comprises an scFv having the configuration from N-terminus to C-terminus, VL—linker—VH. Those of skill in the art would be able to select the appropriate configuration for use in the present invention.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequence es are known in the art, including, without limitation, glycine serine (GS) linkers such as (GS)ₙ, (SG)ₙ, (GSGGS)ₙ (SEQ ID NO: 175), (GGGS)ₙ (SEQ ID NO: 176), and (GGGGS)ₙ (SEQ ID NO: 177), where n represents an integer of at least 1. Exemplary linker sequence es can comprise amino acid sequence es including, without limitation, GGSG (SEQ ID NO:178), GGSGG (SEQ ID NO: 179), GSGSG (SEQ ID NO: 180), GSGGG (SEQ ID NO: 181), GGGSG (SEQ ID NO: 182), GSSSG (SEQ ID NO: 183), GGGGS (SEQ ID NO: 184), GGGGSGGGGSGGGGSGGGGS (SEQ ID NO: 185) and the like. Those of skill in the art would be able to select the appropriate linker sequence for use in the present invention. In one embodiment, an antigen binding domain of the present invention comprises a heavy chain variable region (VH) and a light chain variable region (VL), wherein the VH and VL is separated by the linker sequence e.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequence es as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife et a., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof, or a humanized murine antibody or a fragment thereof.

In certain embodiments, the antigen binding domain comprises a heavy chain variable region that comprises three heavy chain complementarity determining regions (HCDR1, HCDR2, and HCDR3) and a light chain variable region that comprises three light chain complementarity determining regions (LCDR1, LCDR2, and LCDR3).

### Transmembrane Domain

CARs of the present invention may comprise a transmembrane domain that connects the antigen binding domain of the CAR to the intracellular domain of the CAR. The transmembrane domain of the CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence Examples of the transmembrane domain of particular use in this invention include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), ICOS, CD278, Toll-like receptor 1 (TLR1), TLR2, TLR3, TLR4, TLR5, TLR6, TLR7, TLR8, TLR9 or a transmembrane domain derived from a killer immunoglobulin-like receptor (KIR).

In certain embodiments, the transmembrane domain comprises a transmembrane domain of CD8. In certain embodiments, the transmembrane domain of CD8 is a transmembrane domain of CD8α.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in the CAR.

In some embodiments, the transmembrane domain further comprises a hinge region. The CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequence es or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a CAR includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region). In certain embodiments, the hinge region is a CD8a hinge.

The hinge region can have a length of from about 4 amino acids to about 50 amino acids, e.g., from about 4 aa to about 10 aa, from about 10 aa to about 15 aa, from about 15 aa to about aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than amino acids (e.g., 30, 40, 50, 60 or more amino acids).

For example, hinge regions include glycine polymers (G)ₙ, glycine-serine polymers (including, for example, (GS)ₙ, (GSGGS)ₙ and (GGGS)ₙ, where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequence es including, but not limited to, (GGGGS)ₙ (SEQ ID NO: 186), GGSG (SEQ ID NO: 187), GGSGG (SEQ ID NO: 188), GSGSG (SEQ ID NO: 189), GSGGG (SEQ ID NO: 190), GGGSG (SEQ ID NO: 191), GSSSG (SEQ ID NO: 192), GGGGS (SEQ ID NO: 193) and the like.

In some embodiments, the hinge region is an immunoglobulin heavy chain hinge region. Immunoglobulin hinge region amino acid sequence es are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequence es: DKTHT (SEQ ID NO: 194); CPPC (SEQ ID NO: 195); CPEPKSCDTPPPCPR (SEQ ID NO: 196) (see, e.g., Glaser et al., J. Biol. Chem. (2005) 280:41494-41503); ELKTPLGDTTHT (SEQ ID NO: 197); KSCDKTHTCP (SEQ ID NO: 198); KCCVDCP (SEQ ID NO: 199); KYGPPCP (SEQ ID NO: 200); EPKSCDKTHTCPPCP (SEQ ID NO: 201) (human IgG1 hinge); ERKCCVECPPCP (SEQ ID NO: 202) (human IgG2 hinge); ELKTPLGDTTHTCPRCP (SEQ ID NO: 203) (human IgG3 hinge); SPNMVPHAHHAQ (SEQ ID NO: 204) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 205); see, e.g., Yan et al., J. Biol. Chem. (2012) 287: 5891-5897. In one embodiment, the hinge region can comprise an amino acid sequence derived from human CD8, or a variant thereof.

### Intracellular Signaling Domain

A CAR of the present invention also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CDlib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of the CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in a CAR of the present disclosure includes a DAP10/CD28 type signaling chain. In one embodiment, an intracellular signaling domain suitable for use in a CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular domains described herein can be combined with any of the antigen binding domains described herein, any of the transmembrane domains described herein, or any of the other domains described herein that may be included in a CAR.

In certain embodiments, the intracellular domain comprises a costimulatory domain of 4-1BB. In certain embodiments, the intracellular domain comprises an intracellular domain of CD3ζ or a variant thereof. In certain embodiments, the intracellular domain comprises 4-1BB and CD3ζ domians.

Amino acid and nucleotide sequence es of exemplary CARs and CAR domains (e.g., extracellular antigen binding domains which target a tumor antigen) include, but are not limited to, the following:

Human anti-CD19 CAR AA sequence
(SEQ ID NO: 32)
MALPVALLLPLALLLHAARPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITG
GGGSGGGGSGGGGSEVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYYYGGSYAMDYWGQGT
SVTVSSTTTPAPRPPTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGGCELRVKFSRSADAP
AYKQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
Human anti-CD19 CAR NT sequence
(SEQ ID NO: 33)
ATGGCCTTACCAGTGGCCTTGCTCCTGCCGCTGGCCTTGCTGCTCCACGCCGCCAGGCCGGACA
TCCAGATGACACAGACTACATCCTCCCTGTCTGCCTCTCTGGGAGACAGAGTCACCATCAGTTG
CAGGGCAAGTCAGGACATTAGTAAATATTTAAATTGGTATCAGCAGAAACCAGATGGAACTGTT
AAACTCCTGATCTACCATACATCAAGATTACACTCAGGAGTCCCATCAAGGTTCAGTGGCAGTG
GGTCTGGAACAGATTATTCTCTCACCATTAGCAACCTGGAGCAAGAAGATATTGCCACTTACTT
TTGCCAACAGGGTAATACGCTTCCGTACACGTTCGGAGGGGGGACCAAGCTGGAGATCACAGGT
GGCGGTGGCTCGGGCGGTGGTGGGTCGGGTGGCGGCGGATCTGAGGTGAAACTGCAGGAGTCAG
GACCTGGCCTGGTGGCGCCCTCACAGAGCCTGTCCGTCACATGCACTGTCTCAGGGGTCTCATT
ACCCGACTATGGTGTAAGCTGGATTCGCCAGCCTCCACGAAAGGGTCTGGAGTGGCTGGGAGTA
ATATGGGGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAGCCATTTA
CTACTGTGCCAAACATTATTACTACGGTGGTAGCTATGCTATGGACTACTGGGGCCAAGGAACC
TCAGTCACCGTCTCCTCAACCACGACGCCAGCGCCGCGACCACCAACACCGGCGCCCACCATCG
CGTCGCAGCCCCTGTCCCTGCGCCCAGAGGCGTGCCGGCCAGCGGCGGGGGGCGCAGTGCACAC
GAGGGGGCTGGACTTCGCCTGTGATATCTACATCTGGGCGCCCTTGGCCGGGACTTGTGGGGTC
CTTCTCCTGTCACTGGTTATCACCCTTTACTGCAAACGGGGCAGAAAGAAACTCCTGTATATAT
TCAAACAACCATTTATGAGACCAGTACAAACTACTCAAGAGGAAGATGGCTGTAGCTGCCGATT
TCCAGAAGAAGAAGAAGGAGGATGTGAACTGAGAGTGAAGTTCAGCAGGAGCGCAGACGCCCCC
GCGTACAAGCAGGGCCAGAACCAGCTCTATAACGAGCTCAATCTAGGACGAAGAGAGGAGTACG
ATGTTTTGGACAAGAGACGTGGCCGGGACCCTGAGATGGGGGGAAAGCCGAGAAGGAAGAACCC
TCAGGAAGGCCTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTCAGTACAGCCA
CCAAGGACACCTACGACGCCCTTCACATGCAGGCCCTGCCCCCTCGCTAA
Human anti-CD19 CAR
(SEQ ID NO: 34)
MALPVTALLLPLALLLHAARPEIVMTQSPATLSLSPGERATLSCRASQDISKYLNWYQQKPGQA
PRLLIYHTSRLHSGIPARFSGSGSGTDYTLTISSLQPEDFAVYFCQQGNTLPYTFGQGTKLEIK
GGGGSGGGGSGGGGSQVQLQESGPGLVKPSETLSLTCTVSGVSLPDYGVSWIRQPPGKGLEWIG
VIWGSETTYYQSSLKSRVTISKDNSKNQVSLKLSSVTAADTAVYYCAKHYYYGGSYAMDYWGQG
TLVTVSSTTTPAPRPPTPAPTIASQPLSLRPEACRPAAGGAVHTRGLDFACDFWVLVVVGGVLA
CYSLLVTVAFIIFWVRSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
Human anti-CD19 CAR
(SEQ ID NO: 35)
ATGGCCTTACCAGTGACCGCCTTGCTCCTGCCGCTGGCCTTGCTGCTCCACGCCGCCAGGCCGG
AAATTGTGATGACCCAGTCACCCGCCACTCTTAGCCTTTCACCCGGTGAGCGCGCAACCCTGTC
TTGCAGAGCCTCCCAAGACATCTCAAAATACCTTAATTGGTATCAACAGAAGCCCGGACAGGCT
CCTCGCCTTCTGATCTACCACACCAGCCGGCTCCATTCTGGAATCCCTGCCAGGTTCAGCGGTA
GCGGATCTGGGACCGACTACACCCTCACTATCAGCTCACTGCAGCCAGAGGACTTCGCTGTCTA
TTTCTGTCAGCAAGGGAACACCCTGCCCTACACCTTTGGACAGGGCACCAAGCTCGAGATTAAA
GGTGGAGGTGGCAGCGGAGGAGGTGGGTCCGGCGGTGGAGGAAGCCAGGTCCAACTCCAAGAAA
GCGGACCGGGTCTTGTGAAGCCATCAGAAACTCTTTCACTGACTTGTACTGTGAGCGGAGTGTC
TCTCCCCGATTACGGGGTGTCTTGGATCAGACAGCCACCGGGGAAGGGTCTGGAATGGATTGGA
GTGATTTGGGGCTCTGAGACTACTTACTACCAATCATCCCTCAAGTCACGCGTCACCATCTCAA
AGGACAACTCTAAGAATCAGGTGTCACTGAAACTGTCATCTGTGACCGCAGCCGACACCGCCGT
GTACTATTGCGCTAAGCATTACTATTATGGCGGGAGCTACGCAATGGATTACTGGGGACAGGGT
ACTCTGGTCACCGTGTCCAGCACCACGACGCCAGCGCCGCGACCACCAACACCGGCGCCCACCA
TCGCGTCGCAGCCCCTGTCCCTGCGCCCAGAGGCGTGCCGGCCAGCGGCGGGGGGCGCAGTGCA
CACGAGGGGGCTGGACTTCGCCTGTGATTTTTGGGTGCTGGTGGTGGTTGGTGGAGTCCTGGCT
TGCTATAGCTTGCTAGTAACAGTGGCCTTTATTATTTTCTGGGTGAGGAGTAAGAGGAGCAGGC
TCCTGCACAGTGACTACATGAACATGACTCCCCGCCGCCCCGGGCCCACCCGCAAGCATTACCA
GCCCTATGCCCCACCACGCGACTTCGCAGCCTATCGCTCCAGAGTGAAGTTCAGCAGGAGCGCA
GACGCCCCCGCGTACCAGCAGGGCCAGAACCAGCTCTATAACGAGCTCAATCTAGGACGAAGAG
AGGAGTACGATGTTTTGGACAAGAGACGTGGCCGGGACCCTGAGATGGGGGGAAAGCCGAGAAG
GAAGAACCCTCAGGAAGGCCTGTACAATGAACTGCAGAAAGATAAGATGGCGGAGGCCTACAGT
GAGATTGGGATGAAAGGCGAGCGCCGGAGGGGCAAGGGGCACGATGGCCTTTACCAGGGTCTCA
GTACAGCCACCAAGGACACCTACGACGCCCTTCACATGCAGGCCCTGCCCCCTCGC
Murine anti-CD19 CAR AA sequence
(SEQ ID NO: 36)
MASPLTRFLSLNLLLLGESIILGSGEADIQMTQSPASLSTSLGETVTIQCQASEDIYSGLAWYQ
QKPGKSPQLLIYGASDLQDGVPSRFSGSGSGTQYSLKITSMQTEDEGVYFCQQGLTY+NLPRTF
GGGTKLELKGGGGSGGGGSGGGGSEVQLQQSGAELVRPGTSVKLSCKVSGDTITFYYMHFVKQR
PGQGLEWIGRIDPEDESTKYSEKFKNKATLTADTSSNTAYLKLSSLTSEDTATYFCIYGGYYFD
YWGQGVMVTVSSLQKVNSTTTKPVLRTPSPVHPTGTSQPQRPEDCRPRGSVKGTGLDFACDIYF
WALVVVAGVLFCYGLLVTVALCVIWTNSRRNRLLQSDYMNMTPRRPGLTRKPYQPYAPARDFAA
YRPKFSRSAETAANLQDPNQLYNELNLGRREEYDVLEKKRARDPEMGGKQQRRRNPQEGVYNAL
QKDKMAEAYSEIGTKGERRRGKGHDGLYQGLSTATKDTYDALHMQTLAPR
Murine anti-CD19 CAR NT sequence
(SEQ ID NO: 37)
ATGGCCTCACCCCTGACCAGATTCCTGTCACTGAACCTGCTGCTGCTGGGCGAGTCAATCATCC
TGGGCTCAGGCGAGGCCGACATCCAGATGACCCAGAGCCCTGCCAGCCTGTCTACCAGCCTGGG
CGAGACAGTGACCATCCAGTGTCAGGCCAGCGAGGACATCTACTCTGGCCTGGCTTGGTATCAG
CAGAAGCCCGGCAAGAGCCCTCAGCTGCTGATCTACGGCGCCAGCGACCTGCAGGACGGCGTGC
CTAGCAGATTCAGCGGCAGCGGCTCCGGAACCCAGTACAGCCTGAAGATCACCAGCATGCAGAC
CGAGGACGAGGGCGTGTACTTCTGCCAGCAAGGCCTGACCTACCCTAGAACCTTCGGAGGAGGC
ACCAAGCTGGAACTGAAGGGCGGAGGCGGAAGTGGAGGCGGAGGATCTGGCGGCGGAGGCTCTG
AAGTGCAGCTGCAGCAGTCTGGCGCTGAACTGGTCCGGCCTGGCACTAGCGTGAAGCTGTCCTG
CAAGGTGTCCGGCGACACCATCACCTTCTACTACATGCACTTCGTGAAGCAGAGGCCAGGACAG
GGCCTGGAATGGATCGGCAGAATCGACCCTGAGGACGAGAGCACCAAGTACAGCGAGAAGTTCA
AGAACAAGGCCACCCTGACCGCCGACACCAGCAGCAACACCGCCTACCTGAAGCTGTCTAGCCT
GACCTCCGAGGACACCGCCACCTACTTTTGCATCTACGGCGGCTACTACTTCGACTACTGGGGC
CAGGGCGTGATGGTCACCGTGTCCAGCCTGCAGAAGGTGAACTCAACCACCACCAAGCCCGTGC
TGAGAACCCCCTCACCCGTGCACCCCACCGGCACCTCACAGCCCCAGAGACCCGAGGACTGCAG
ACCCAGAGGCTCAGTGAAGGGCACCGGCCTGGACTTCGCCTGCGACATCTACTTTTGGGCACTG
GTCGTGGTTGCTGGAGTCCTGTTTTGTTATGGCTTGCTAGTGACAGTGGCTCTTTGTGTTATCT
GGACAAATAGTAGAAGGAACAGACTCCTTCAAAGTGACTACATGAACATGACTCCCCGGAGGCC
TGGGCTCACTCGAAAGCCTTACCAGCCCTACGCCCCTGCCAGAGACTTTGCAGCGTACCGCCCC
AAGTTCTCAAGATCAGCCGAGACCGCCGCCAACCTGCAGGACCCCAACCAGCTGTACAACGAGC
TGAACCTGGGCAGAAGAGAGGAGTACGACGTGCTGGAGAAGAAGAGAGCCAGAGACCCCGAGAT
GGGCGGCAAGCAGCAGAGAAGAAGAAACCCCCAGGAGGGCGTGTACAACGCCCTGCAGAAGGAC
AAGATGGCCGAGGCCTACTCAGAGATCGGCACCAAGGGCGAGAGAAGAAGAGGCAAGGGCCACG
ACGGCCTGTACCAGGGCCTGTCAACCGCCACCAAGGACACCTACGACGCCCTGCACATGCAGAC
CCTGGCCCCCAGA
Murine anti-MSLN CAR AA sequence
(SEQ ID NO: 38)
MASPLTRFLSLNLLLLGESIILGSGEAAQVQLQESGPGLVKPSQTLSLTCTVSGGSISSGGYYW
SWIRQHPGKGLEWIGYIYYSGSTYYNPSLKSRVTISVDTSKNQFSLKLSSVTAADTAVYYCARF
DYGDFYDAFDIWGQGTMVTVSSGGGGSGGGGSGGGGSEIVLTQSPSSLSASVGDRVTITCRASQ
GISSALAWYQQKPGKAPKLLIYDASSLESGVPSRFSGSGSGTDFTLTISSLQPEDFATYYCQQF
NSYPITFGQGTRLEIKRLQKVNSTTTKPVLRTPSPVHPTGTSQPQRPEDCRPRGSVKGTGLDFA
CDIYIWAPLAGICVALLLSLIITLIKWIRKKFPHIFKQPFKKTTGAAQEEDACSCRCPQEEEGG
GGGYELKFSRSAETAANLQDPNQLYNELNLGRREEYDVLEKKRARDPEMGGKQQRRRNPQEGVY
NALQKDKMAEAYSEIGTKGERRRGKGHDGLYQGLSTATKDTYDALHMQTLAPR
Murine anti-MSLN CAR NT sequence
(SEQ ID NO: 39)
ATGGCCAGCCCCCTGACCAGATTCCTGAGCCTGAACCTGCTGCTGCTGGGCGAGAGCATCATCC
TGGGCAGCGGCGAGGCCGCCCAGGTGCAGCTGCAGGAGTCGGGCCCAGGACTGGTGAAGCCTTC
ACAGACCCTGTCCCTCACCTGCACTGTCTCTGGTGGCTCCATCAGCAGTGGTGGTTACTACTGG
AGCTGGATCCGCCAGCACCCAGGGAAGGGCCTGGAGTGGATTGGGTACATCTATTACAGTGGGA
GCACCTACTACAACCCGTCCCTCAAGAGTCGAGTTACCATATCAGTAGACACGTCCAAGAACCA
GTTCTCCCTGAAGCTGAGCTCTGTGACCGCTGCGGACACGGCCGTGTATTACTGTGCGAGATTT
GACTACGGTGACTTCTATGATGCTTTTGATATCTGGGGCCAAGGGACAATGGTCACCGTCTCTT
CAGGTGGTGGTGGTAGCGGCGGCGGCGGCTCTGGTGGTGGTGGATCCGAAATTGTGTTGACGCA
GTCTCCATCCTCCCTGTCTGCATCTGTAGGAGACAGAGTCACCATCACTTGCCGGGCAAGTCAG
GGCATTAGCAGTGCTTTAGCCTGGTATCAGCAGAAACCAGGGAAAGCTCCTAAGCTCCTGATCT
ATGATGCCTCCAGTTTGGAAAGTGGGGTCCCATCAAGGTTCAGCGGCAGTGGATCTGGGACAGA
TTTCACTCTCACCATCAGCAGCCTGCAGCCTGAAGATTTTGCAACTTATTACTGTCAACAGTTT
AATAGTTACCCGATCACCTTCGGCCAAGGGACACGACTGGAGATTAAACGTCTGCAGAAGGTGA
ACAGCACCACCACCAAGCCCGTGCTGAGAACCCCCAGCCCCGTGCACCCCACCGGCACCAGCCA
GCCCCAGAGACCCGAGGACTGCAGACCCAGAGGCAGCGTGAAGGGCACCGGCCTGGACTTCGCC
TGCGACATCTACATCTGGGCCCCCCTGGCCGGCATCTGCGTGGCCCTGCTGCTGAGCCTGATCA
TCACCCTGATCAAGTGGATCAGAAAGAAGTTCCCCCACATCTTCAAGCAGCCCTTCAAGAAGAC
CACCGGCGCCGCCCAGGAGGAGGACGCCTGCAGCTGCAGATGCCCCCAGGAGGAGGAGGGCGGC
GGCGGCGGCTACGAGCTGAAGTTCAGCAGAAGCGCCGAGACCGCCGCCAACCTGCAGGACCCCA
ACCAGCTGTACAACGAGCTGAACCTGGGCAGAAGAGAGGAGTACGACGTGCTGGAGAAGAAGAG
AGCCAGAGACCCCGAGATGGGCGGCAAGCAGCAGAGAAGAAGAAACCCCCAGGAGGGCGTGTAC
AACGCCCTGCAGAAGGACAAGATGGCCGAGGCCTACAGCGAGATCGGCACCAAGGGCGAGAGAA
GAAGAGGCAAGGGCCACGACGGCCTGTACCAGGGCCTGAGCACCGCCACCAAGGACACCTACGA
CGCCCTGCACATGCAGACCCTGGCCCCCAGA
Human anti-MSLN CAR AA sequence
(SEQ ID NO: 40)
MALPVTALLLPLALLLHAARPQVQLVQSGAEVEKPGASVKVSCKASGYTFTDYYMHWVRQAPGQ
GLEWMGWINPNSGGTNYAQKFQGRVTMTRDTSISTAYMELSRLRSDDTAVYYCASGWDFDYWGQ
GTLVTVSSGGGGSGGGGSGGGGSGGGGSDIVMTQSPSSLSASVGDRVTITCRASQSIRYYLSWY
QQKPGKAPKLLIYTASILQNGVPSRFSGSGSGTDFTLTISSLQPEDFATYYCLQTYTTPDFGPG
TKVEIKTTTPAPRPPTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGGCELRVKFSRSADAP
AYKQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
Human anti-MSLN CAR NT sequence
(SEQ ID NO: 41)
ATGGCCCTCCCTGTCACCGCCCTGCTGCTTCCGCTGGCTCTTCTGCTCCACGCCGCTCGGCCCC
AAGTCCAACTCGTTCAATCAGGCGCAGAAGTCGAAAAGCCCGGAGCATCAGTCAAAGTCTCTTG
CAAGGCTTCCGGCTACACCTTCACGGACTACTACATGCACTGGGTGCGCCAGGCTCCAGGCCAG
GGACTGGAGTGGATGGGATGGATCAACCCGAATTCCGGGGGAACTAACTACGCCCAGAAGTTTC
AGGGCCGGGTGACTATGACTCGCGATACCTCGATCTCGACTGCGTACATGGAGCTCAGCCGCCT
CCGGTCGGACGATACCGCCGTGTACTATTGTGCGTCGGGATGGGACTTCGACTACTGGGGGCAG
GGCACTCTGGTCACTGTGTCAAGCGGAGGAGGTGGATCAGGTGGAGGTGGAAGCGGGGGAGGAG
GTTCCGGCGGCGGAGGATCAGATATCGTGATGACGCAATCGCCTTCCTCGTTGTCCGCATCCGT
GGGAGACAGGGTGACCATTACTTGCAGAGCGTCCCAGTCCATTCGGTACTACCTGTCGTGGTAC
CAGCAGAAGCCGGGGAAAGCCCCAAAACTGCTTATCTATACTGCCTCGATCCTCCAAAACGGCG
TGCCATCAAGATTCAGCGGTTCGGGCAGCGGGACCGACTTTACCCTGACTATCAGCAGCCTGCA
GCCGGAAGATTTCGCCACGTACTACTGCCTGCAAACCTACACCACCCCGGACTTCGGACCTGGA
ACCAAGGTGGAGATCAAGACCACTACCCCAGCACCGAGGCCACCCACCCCGGCTCCTACCATCG
CCTCCCAGCCTCTGTCCCTGCGTCCGGAGGCATGTAGACCCGCAGCTGGTGGGGCCGTGCATAC
CCGGGGTCTTGACTTCGCCTGCGATATCTACATTTGGGCCCCTCTGGCTGGTACTTGCGGGGTC
CTGCTGCTTTCACTCGTGATCACTCTTTACTGTAAGCGCGGTCGGAAGAAGCTGCTGTACATCT
TTAAGCAACCCTTCATGAGGCCTGTGCAGACTACTCAAGAGGAGGACGGCTGTTCATGCCGGTT
CCCAGAGGAGGAGGAAGGCGGCTGCGAACTGCGCGTGAAATTCAGCCGCAGCGCAGATGCTCCA
GCCTACAAGCAGGGGCAGAACCAGCTCTACAACGAACTCAATCTTGGTCGGAGAGAGGAGTACG
ACGTGCTGGACAAGCGGAGAGGACGGGACCCAGAAATGGGCGGGAAGCCGCGCAGAAAGAATCC
CCAAGAGGGCCTGTACAACGAGCTCCAAAAGGATAAGATGGCAGAAGCCTATAGCGAGATTGGT
ATGAAAGGGGAACGCAGAAGAGGCAAAGGCCACGACGGACTGTACCAGGGACTCAGCACCGCCA
CCAAGGACACCTATGACGCTCTTCACATGCAGGCCCTGCCGCCTCGG
A03 Murine anti-MSLN scFv nucleotide sequence
(SEQ ID NO: 42)
GCCCAGGTGCAGCTGCAGGAGTCGGGCCCAGGACTGGTGAAGCCTTCACAGACCCTGTCCCTCA
CCTGCACTGTCTCTGGTGGCTCCATCAGCAGTGGTGGTTACTACTGGAGCTGGATCCGCCAGCA
CCCAGGGAAGGGCCTGGAGTGGATTGGGTACATCTATTACAGTGGGAGCACCTACTACAACCCG
TCCCTCAAGAGTCGAGTTACCATATCAGTAGACACGTCCAAGAACCAGTTCTCCCTGAAGCTGA
GCTCTGTGACCGCTGCGGACACGGCCGTGTATTACTGTGCGAGATTTGACTACGGTGACTTCTA
TGATGCTTTTGATATCTGGGGCCAAGGGACAATGGTCACCGTCTCTTCAGGTGGTGGTGGTAGC
GGCGGCGGCGGCTCTGGTGGTGGTGGATCCGAAATTGTGTTGACGCAGTCTCCATCCTCCCTGT
CTGCATCTGTAGGAGACAGAGTCACCATCACTTGCCGGGCAAGTCAGGGCATTAGCAGTGCTTT
AGCCTGGTATCAGCAGAAACCAGGGAAAGCTCCTAAGCTCCTGATCTATGATGCCTCCAGTTTG
GAAAGTGGGGTCCCATCAAGGTTCAGCGGCAGTGGATCTGGGACAGATTTCACTCTCACCATCA
GCAGCCTGCAGCCTGAAGATTTTGCAACTTATTACTGTCAACAGTTTAATAGTTACCCGATCAC
CTTCGGCCAAGGGACACGACTGGAGATTAAACGT
A03 Murine anti-MSLN scFv amino acid sequence
(SEQ ID NO: 43)
AQVQLQESGPGLVKPSQTLSLTCTVSGGSISSGGYYWSWIRQHPGKGLEWIGYIYYSGSTYYNP
SLKSRVTISVDTSKNQFSLKLSSVTAADTAVYYCARFDYGDFYDAFDIWGQGTMVTVSSGGGGS
GGGGSGGGGSEIVLTQSPSSLSASVGDRVTITCRASQGISSALAWYQQKPGKAPKLLIYDASSL
ESGVPSRFSGSGSGTDFTLTISSLQPEDFATYYCQQFNSYPITFGQGTRLEIKR
M5 Human anti-MSLN scFv nucleotide sequence
(SEQ ID NO: 44)
CAAGTCCAACTCGTTCAATCAGGCGCAGAAGTCGAAAAGCCCGGAGCATCAGTCAAAGTCTCTT
GCAAGGCTTCCGGCTACACCTTCACGGACTACTACATGCACTGGGTGCGCCAGGCTCCAGGCCA
GGGACTGGAGTGGATGGGATGGATCAACCCGAATTCCGGGGGAACTAACTACGCCCAGAAGTTT
CAGGGCCGGGTGACTATGACTCGCGATACCTCGATCTCGACTGCGTACATGGAGCTCAGCCGCC
TCCGGTCGGACGATACCGCCGTGTACTATTGTGCGTCGGGATGGGACTTCGACTACTGGGGGCA
GGGCACTCTGGTCACTGTGTCAAGCGGAGGAGGTGGATCAGGTGGAGGTGGAAGCGGGGGAGGA
GGTTCCGGCGGCGGAGGATCAGATATCGTGATGACGCAATCGCCTTCCTCGTTGTCCGCATCCG
TGGGAGACAGGGTGACCATTACTTGCAGAGCGTCCCAGTCCATTCGGTACTACCTGTCGTGGTA
CCAGCAGAAGCCGGGGAAAGCCCCAAAACTGCTTATCTATACTGCCTCGATCCTCCAAAACGGC
GTGCCATCAAGATTCAGCGGTTCGGGCAGCGGGACCGACTTTACCCTGACTATCAGCAGCCTGC
AGCCGGAAGATTTCGCCACGTACTACTGCCTGCAAACCTACACCACCCCGGACTTCGGACCTGG
AACCAAGGTGGAGATCAAG
M5 Human anti-MSLN scFv amino acid sequence
(SEQ ID NO: 45)
QVQLVQSGAEVEKPGASVKVSCKASGYTFTDYYMHWVRQAPGQGLEWMGWINPNSGGTNYAQKF
QGRVTMTRDTSISTAYMELSRLRSDDTAVYYCASGWDFDYWGQGTLVTVSSGGGGSGGGGSGGG
GSGGGGSDIVMTQSPSSLSASVGDRVTITCRASQSIRYYLSWYQQKPGKAPKLLIYTASILQNG
VPSRFSGSGSGTDFTLTISSLQPEDFATYYCLQTYTTPDFGPGTKVEIK
Anti-GD2 scFv nucleotide sequence
(SEQ ID NO: 46)
ATGGCCTTACCAGTGACCGCCTTGCTCCTGCCGCTGGCCTTGCTGCTCCACGCCGCCAGGCCGG
GATCCGATGTTGTGATGACCCAAACTCCACTCTCCCTGCCTGTCAGTCTTGGAGATCAAGCCTC
CATCTCTTGCAGATCTAGTCAGAGTCTTGTACACCGTAACGGAAACACCTATTTACATTGGTAC
CTGCAGAAGCCAGGCCAGTCTCCAAAGCTCCTGATTCACAAAGTTTCCAACCGATTTTCTGGGG
TCCCAGACAGGTTCAGTGGCAGTGGATCAGGGACAGATTTCACACTCAAGATCAGCAGAGTGGA
GGCTGAGGATCTGGGAGTTTATTTCTGTTCTCAAAGTACACACGTTCCTCCGCTCACGTTCGGT
GCTGGGACCAAGCTGGAGCTGAAAGGAGGTGGCGGGTCAGGGGGTGGCGGAAGCGGAGGCGGCG
GTTCAGGCGGAGGAGGCTCGGAGGTGCAGCTTCTGCAGTCTGGACCTGAGCTGGAGAAGCCTTC
CGCTTCAGTGATGATATCCTGCAAGGCTTCTGGTTCCTCCTTCACTGGCTACAACATGAACTGG
GTGAGGCAGAATATTGGAAAGAGCCTTGAATGGATTGGAGCTATTGATCCTTACTACGGTGGAA
CTAGCTACAACCAGAAGTTCAAGGGCAGGGCCACATTGACTGTAGACAAATCGTCCAGCACAGC
CTACATGCACCTCAAGAGCCTGACATCTGAGGACTCTGTCTATTACTGTGTAAGCGGAATGGAG
TACTGGGGTCAAGGAACCTCAGTCACCGTCTCCTCATCCGGA
Anti-GD2 scFv amino acid sequence
(SEQ ID NO: 47)
MALPVTALLLPLALLLHAARPGSDVVMTQTPLSLPVSLGDQASISCRSSQSLVHRNGNTYLHWY
LQKPGQSPKLLIHKVSNRFSGVPDRFSGSGSGTDFTLKISRVEAEDLGVYFCSQSTHVPPLTFG
AGTKLELKGGGGSGGGGSGGGGSGGGGSEVQLLQSGPELEKPSASVMISCKASGSSFTGYNMNW
VRONIGKSLEWIGAIDPYYGGTSYNQKFKGRATLTVDKSSSTAYMHLKSLTSEDSVYYCVSGME
YWGQGTSVTVSSSG
Anti-HER2 scFv (high affinity) nucleotide sequence
(SEQ ID NO: 48)
ATGGATTTTCAGGTGCAGATTTTCAGCTTCCTGCTAATCAGTGCCTCAGTCATAATGTCCAGAG
GAGATATCCAGATGACCCAGTCCCCGAGCTCCCTGTCCGCCTCTGTGGGCGATAGGGTCACCAT
CACCTGCCGTGCCAGTCAGGATGTGAATACTGCTGTAGCCTGGTATCAACAGAAACCAGGAAAA
GCTCCGAAACTACTGATTTACTCGGCATCCTTCCTTTATTCTGGAGTCCCTTCTCGCTTCTCTG
GATCTAGATCTGGGACGGATTTCACTCTGACCATCAGCAGTCTGCAGCCGGAAGACTTCGCAAC
TTATTACTGTCAGCAACATTATACTACTCCTCCCACGTTCGGACAGGGTACCAAGGTGGAGATC
AAACGCACTGGGTCTACATCTGGATCTGGGAAGCCGGGTTCTGGTGAGGGTTCTGAGGTTCAGC
TGGTGGAGTCTGGCGGTGGCCTGGTGCAGCCAGGGGGCTCACTCCGTTTGTCCTGTGCAGCTTC
TGGCTTCAACATTAAAGACACCTATATACACTGGGTGCGTCAGGCCCCGGGTAAGGGCCTGGAA
TGGGTTGCAAGGATTTATCCTACGAATGGTTATACTAGATATGCCGATAGCGTCAAGGGCCGTT
TCACTATAAGCGCAGACACATCCAAAAACACAGCCTACCTGCAGATGAACAGCCTGCGTGCTGA
GGACACTGCCGTCTATTATTGTTCTAGATGGGGAGGGGACGGCTTCTATGCTATGGACGTGTGG
GGTCAAGGAACCCTGGTCACCGTCTCCTCG
Anti-HER2 scFv (high affinity) amino acid sequence
(SEQ ID NO: 49)
MDFQVQIFSFLLISASVIMSRGDIQMTQSPSSLSASVGDRVTITCRASQDVNTAVAWYQQKPGK
APKLLIYSASFLYSGVPSRFSGSRSGTDFTLTISSLQPEDFATYYCQQHYTTPPTFGQGTKVEI
KRTGSTSGSGKPGSGEGSEVOLVESGGGLVQPGGSLRLSCAASGFNIKDTYIHWVRQAPGKGLE
WVARIYPTNGYTRYADSVKGRFTISADTSKNTAYLQMNSLRAEDTAVYYCSRWGGDGFYAMDVW
GOGTLVTVSS
Anti-HER2 scFv (low affinity) nucleotide sequence
(SEQ ID NO: 50)
ATGGATTTTCAGGTGCAGATTTTCAGCTTCCTGCTAATCAGTGCCTCAGTCATAATGTCCAGAG
GAGATATCCAGATGACCCAGTCCCCGAGCTCCCTGTCCGCCTCTGTGGGCGATAGGGTCACCAT
CACCTGCCGTGCCAGTCAGGATGTGAATACTGCTGTAGCCTGGTATCAACAGAAACCAGGAAAA
GCTCCGAAACTACTGATTTACTCGGCATCCTTCCTTGAGTCTGGAGTCCCTTCTCGCTTCTCTG
GATCTAGATCTGGGACGGATTTCACTCTGACCATCAGCAGTCTGCAGCCGGAAGACTTCGCAAC
TTATTACTGTCAGCAACATTATACTACTCCTCCCACGTTCGGACAGGGTACCAAGGTGGAGATC
AAACGCACTGGGTCTACATCTGGATCTGGGAAGCCGGGTTCTGGTGAGGGTTCTGAGGTTCAGC
TGGTGGAGTCTGGCGGTGGCCTGGTGCAGCCAGGGGGCTCACTCCGTTTGTCCTGTGCAGCTTC
TGGCTTCAACATTAAAGACACCTATATACACTGGGTGCGTCAGGCCCCGGGTAAGGGCCTGGAA
TGGGTTGCAAGGATTTATCCTACGAATGGTTATACTAGATATGCCGATAGCGTCAAGGGCCGTT
TCACTATAAGCGCAGACACATCCAAAAACACAGCCTACCTGCAGATGAACAGCCTGCGTGCTGA
GGACACTGCCGTCTATTATTGTTCTAGATGGGGAGGGGACGGCTTCGTTGCTATGGACGTGTGG
GGTCAAGGAACCCTGGTCACCGTCTCCTCG
Anti-HER2 scFv (low affinity) amino acid sequence
(SEQ ID NO: 51)
MDFQVQIFSFLLISASVIMSRGDIQMTQSPSSLSASVGDRVTITCRASQDVNTAVAWYQQKPGK
APKLLIYSASFLESGVPSRFSGSRSGTDFTLTISSLQPEDFATYYCQQHYTTPPTFGQGTKVEI
KRTGSTSGSGKPGSGEGSEVOLVESGGGLVQPGGSLRLSCAASGFNIKDTYIHWVRQAPGKGLE
WVARIYPTNGYTRYADSVKGRFTISADTSKNTAYLQMNSLRAEDTAVYYCSRWGGDGFVAMDVW
GOGTLVTVSS
Anti-TnMuc1 scFv nucleotide sequence
(SEQ ID NO: 52)
CAGGTGCAGCTGCAGCAGTCTGATGCCGAGCTCGTGAAGCCTGGCAGCAGCGTGAAGATCAGCT
GCAAGGCCAGCGGCTACACCTTCACCGACCACGCCATCCACTGGGTCAAGCAGAAGCCTGAGCA
GGGCCTGGAGTGGATCGGCCACTTCAGCCCCGGCAACACCGACATCAAGTACAACGACAAGTTC
AAGGGCAAGGCCACCCTGACCGTGGACAGAAGCAGCAGCACCGCCTACATGCAGCTGAACAGCC
TGACCAGCGAGGACAGCGCCGTGTACTTCTGCAAGACCAGCACCTTCTTTTTCGACTACTGGGG
CCAGGGCACAACCCTGACAGTGTCTAGCGGAGGCGGAGGATCTGGCGGCGGAGGAAGTGGCGGA
GGGGGATCTGAACTCGTGATGACCCAGAGCCCCAGCTCTCTGACAGTGACAGCCGGCGAGAAAG
TGACCATGATCTGCAAGTCCTCCCAGAGCCTGCTGAACTCCGGCGACCAGAAGAACTACCTGAC
CTGGTATCAGCAGAAACCCGGCCAGCCCCCCAAGCTGCTGATCTTTTGGGCCAGCACCCGGGAA
AGCGGCGTGCCCGATAGATTCACAGGCAGCGGCTCCGGCACCGACTTTACCCTGACCATCAGCT
CCGTGCAGGCCGAGGACCTGGCCGTGTATTACTGCCAGAACGACTACAGCTACCCCCTGACCTT
CGGAGCCGGCACCAAGCTGGAACTGAAG
Anti-TnMuc1 scFv amino acid sequence
(SEQ ID NO: 53)
QVQLQQSDAELVKPGSSVKISCKASGYTFTDHAIHWVKQKPEQGLEWIGHFSPGNTDIKYNDKF
KGKATLTVDRSSSTAYMQLNSLTSEDSAVYFCKTSTFFFDYWGQGTTLTVSSGGGGSGGGGSGG
GGSELVMTQSPSSLTVTAGEKVTMICKSSQSLLNSGDQKNYLTWYQQKPGQPPKLLIFWASTRE
SGVPDRFTGSGSGTDFTLTISSVQAEDLAVYYCONDYSYPLTFGAGTKLELK
Anti-PSMA scFv nucleotide sequence
(SEQ ID NO: 54)
ATGGCCCTGCCTGTGACAGCCCTGCTGCTGCCTCTGGCTCTGCTGCTGCACGCCGCCAGACCTG
GATCTGACATTGTGATGACCCAGTCTCACAAATTCATGTCCACATCAGTAGGAGACAGGGTCAG
CATCATCTGTAAGGCCAGTCAAGATGTGGGTACTGCTGTAGACTGGTATCAACAGAAACCAGGA
CAATCTCCTAAACTACTGATTTATTGGGCATCCACTCGGCACACTGGAGTCCCTGATCGCTTCA
CAGGCAGTGGATCTGGGACAGACTTCACTCTCACCATTACTAACGTTCAGTCTGAAGACTTGGC
AGATTATTTCTGTCAGCAATATAACAGCTATCCTCTCACGTTCGGTGCTGGGACCATGCTGGAC
CTGAAAGGAGGCGGAGGATCTGGCGGCGGAGGAAGTTCTGGCGGAGGCAGCGAGGTGCAGCTGC
AGCAGAGCGGACCCGAGCTCGTGAAGCCTGGAACAAGCGTGCGGATCAGCTGCAAGACCAGCGG
CTACACCTTCACCGAGTACACCATCCACTGGGTCAAGCAGTCCCACGGCAAGAGCCTGGAGTGG
ATCGGCAATATCAACCCCAACAACGGCGGCACCACCTACAACCAGAAGTTCGAGGACAAGGCCA
CCCTGACCGTGGACAAGAGCAGCAGCACCGCCTACATGGAACTGCGGAGCCTGACCAGCGAGGA
CAGCGCCGTGTACTATTGTGCCGCCGGTTGGAACTTCGACTACTGGGGCCAGGGCACAACCCTG
ACAGTGTCTAGCGCTAGCTCCGGA
Anti-PSMA scFv amino acid sequence
(SEQ ID NO: 55)
MALPVTALLLPLALLLHAARPGSDIVMTQSHKFMSTSVGDRVSIICKASQDVGTAVDWYQQKPG
QSPKLLIYWASTRHTGVPDRFTGSGSGTDFTLTITNVQSEDLADYFCQQYNSYPLTFGAGTMLD
LKGGGGSGGGGSSGGGSEVQLQQSGPELVKPGTSVRISCKTSGYTFTEYTIHWVKQSHGKSLEW
IGNINPNNGGTTYNQKFEDKATLTVDKSSSTAYMELRSLTSEDSAVYYCAAGWNFDYWGQGTTL
TVSSASSG
Anti-EGFRvIII scFv nucleotide sequence
(SEQ ID NO: 56)
ATGGCCCTCCCTGTCACCGCCCTGCTGCTTCCGCTGGCTCTTCTGCTCCACGCCGCTCGGCCCG
AGATTCAGCTCGTGCAATCGGGAGCGGAAGTCAAGAAGCCAGGAGAGTCCTTGCGGATCTCATG
CAAGGGTAGCGGCTTTAACATCGAGGATTACTACATCCACTGGGTGAGGCAGATGCCGGGGAAG
GGACTCGAATGGATGGGACGGATCGACCCAGAAAACGACGAAACTAAGTACGGTCCGATCTTCC
AAGGCCATGTGACTATTAGCGCCGATACTTCAATCAATACCGTGTATCTGCAATGGTCCTCATT
GAAAGCCTCAGATACCGCGATGTACTACTGTGCTTTCAGAGGAGGGGTCTACTGGGGACAGGGA
ACTACCGTGACTGTCTCGTCCGGCGGAGGCGGGTCAGGAGGTGGCGGCAGCGGAGGAGGAGGGT
CCGGCGGAGGTGGGTCCGACGTCGTGATGACCCAGAGCCCTGACAGCCTGGCAGTGAGCCTGGG
CGAAAGAGCTACCATTAACTGCAAATCGTCGCAGAGCCTGCTGGACTCGGACGGAAAAACGTAC
CTCAATTGGCTGCAGCAAAAGCCTGGCCAGCCACCGAAGCGCCTTATCTCACTGGTGTCGAAGC
TGGATTCGGGAGTGCCCGATCGCTTCTCCGGCTCGGGATCGGGTACTGACTTCACCCTCACTAT
CTCCTCGCTTCAAGCAGAGGACGTGGCCGTCTACTACTGCTGGCAGGGAACCCACTTTCCGGGA
ACCTTCGGCGGAGGGACGAAAGTGGAGATCAAG
Anti-EGFRvIII scFv amino acid sequence
(SEQ ID NO: 57)
MALPVTALLLPLALLLHAARPEIQLVQSGAEVKKPGESLRISCKGSGFNIEDYYIHWVRQMPGK
GLEWMGRIDPENDETKYGPIFQGHVTISADTSINTVYLQWSSLKASDTAMYYCAFRGGVYWGQG
TTVTVSSGGGGSGGGGSGGGGSGGGGSDVVMTQSPDSLAVSLGERATINCKSSQSLLDSDGKTY
LNWLQQKPGQPPKRLISLVSKLDSGVPDRFSGSGSGTDFTLTISSLQAEDVAVYYCWQGTHFPG
TFGGGTKVEIK
Anti-FAP scFv nucleotide sequence
(SEQ ID NO: 58)
CAAATTGTTCTCACCCAGTCTCCAGCGCTCATGTCTGCTTCTCCAGGGGAGAAGGTCACCATGA
CCTGCACTGCCAGCTCAAGTGTTAGTTACATGTACTGGTACCAGCAGAAGCCACGATCCTCCCC
CAAACCCTGGATTTTTCTCACCTCCAACCTGGCTTCTGGAGTCCCTGCTCGCTTCAGTGGCCGT
GGGTCTGGGACCTCTTTCTCTCTCACAATCAGCAGCAT+NLGGAGGCTGAAGATGCTGCCACTT
ATTACTGCCAGCAGTGGAGTGGTTACCCACCCATCACATTCGGCTCGGGGACAAAGTTGGAAAT
AAAAGGTGGAGGTGGCAGCGGAGGAGGTGGGTCCGGCGGTGGAGGAAGCCAGGTCCAACTGCAG
CAGCCTGGGGCTGAACTGGTAAAGCCTGGGGCTTCAGTGAAGTTGTCCTGCAAGGCGTCTGGCT
ACACCATCACCAGCTACTCTCTGCACTGGGTGAAGCAGAGGCCTGGACAAGGCCTTGAGTGGAT
TGGAGAGATTAATCCTGCCAATGGTGATCATAACTTCAGTGAGAAGTTCGAGATCAAGGCCACA
CTGACTGTAGACAGCTCCTCCAACACAGCATTCATGCAACTCAGCAGGCTGACATCTGAGGACT
CTGCGGTCTATTACTGTACAAGATTGGACGATAGTAGGTTCCACTGGTACTTCGATGTCTGGGG
CGCAGGGACCACGGTCACCGTCTCCTCA
Anti-FAP scFv amino acid sequence
(SEQ ID NO: 59)
QIVLTQSPALMSASPGEKVTMTCTASSSVSYMYWYQQKPRSSPKPWIFLTSNLASGVPARFSGR
GSGTSFSLTISSMEAEDAATYYCQQWSGYPPITFGSGTKLEIKGGGGSGGGGSGGGGSQVQLQQ
PGAELVKPGASVKLSCKASGYTITSYSLHWVKQRPGQGLEWIGEINPANGDHNFSEKFEIKATL
TVDSSSNTAFMQLSRLTSEDSAVYYCTRLDDSRFHWYFDVWGAGTTVTVSS
Mouse CD8 Leader
(SEQ ID NO: 60)
MASPLTRFLSLNLLLLGESIILGSGEA
Mouse CD8 Leader
(SEQ ID NO: 61)
ATGGCCTCACCCCTGACCAGATTCCTGTCACTGAACCTGCTGCTGCTGGGCGAGTCAATCATCC
TGGGCTCAGGCGAGGCC
Mouse anti-mCD19 scFv
(SEQ ID NO: 62)
DIQMTQSPASLSTSLGETVTIQCQASEDIYSGLAWYQQKPGKSPQLLIYGASDLQDGVPSRFSG
SGSGTQYSLKITSMQTEDEGVYFCQQGLTYPRTFGGGTKLELKGGGGSGGGGSGGGGSEVQLQQ
SGAELVRPGTSVKLSCKVSGDTITFYYMHFVKQRPGQGLEWIGRIDPEDESTKYSEKFKNKATL
TADTSSNTAYLKLSSLTSEDTATYFCIYGGYYFDYWGQGVMVTVSS
Mouse anti-CD19 scFv
(SEQ ID NO: 63)
GACATCCAGATGACCCAGAGCCCTGCCAGCCTGTCTACCAGCCTGGGCGAGACAGTGACCATCC
AGTGTCAGGCCAGCGAGGACATCTACTCTGGCCTGGCTTGGTATCAGCAGAAGCCCGGCAAGAG
CCCTCAGCTGCTGATCTACGGCGCCAGCGACCTGCAGGACGGCGTGCCTAGCAGATTCAGCGGC
AGCGGCTCCGGAACCCAGTACAGCCTGAAGATCACCAGCATGCAGACCGAGGACGAGGGCGTGT
ACTTCTGCCAGCAAGGCCTGACCTACCCTAGAACCTTCGGAGGAGGCACCAAGCTGGAACTGAA
GGGCGGAGGCGGAAGTGGAGGCGGAGGATCTGGCGGCGGAGGCTCTGAAGTGCAGCTGCAGCAG
TCTGGCGCTGAACTGGTCCGGCCTGGCACTAGCGTGAAGCTGTCCTGCAAGGTGTCCGGCGACA
CCATCACCTTCTACTACATGCACTTCGTGAAGCAGAGGCCAGGACAGGGCCTGGAATGGATCGG
CAGAATCGACCCTGAGGACGAGAGCACCAAGTACAGCGAGAAGTTCAAGAACAAGGCCACCCTG
ACCGCCGACACCAGCAGCAACACCGCCTACCTGAAGCTGTCTAGCCTGACCTCCGAGGACACCG
CCACCTACTTTTGCATCTACGGCGGCTACTACTTCGACTACTGGGGCCAGGGCGTGATGGTCAC
CGTGTCCAGC
Mouse CD8 Hinge
(SEQ ID NO: 64)
LQKVNSTTTKPVLRTPSPVHPTGTSQPQRPEDCRPRGSVKGTGLDFACDIY
Mouse CD8 Hinge
(SEQ ID NO: 65)
CTGCAGAAGGTGAACTCAACCACCACCAAGCCCGTGCTGAGAACCCCCTCACCCGTGCACCCCA
CCGGCACCTCACAGCCCCAGAGACCCGAGGACTGCAGACCCAGAGGCTCAGTGAAGGGCACCGG
CCTGGACTTCGCCTGCGACATCTAC
Mouse CD28 TM
(SEQ ID NO: 66)
FWALVVVAGVLFCYGLLVTVALCVIWT
Mouse CD28 TM
(SEQ ID NO: 67)
TTTTGGGCACTGGTCGTGGTTGCTGGAGTCCTGTTTTGTTATGGCTTGCTAGTGACAGTGGCTC
TTTGTGTTATCTGGACA
Mouse CD28 ICD
(SEQ ID NO: 68)
NSRRNRLLQSDYMNMTPRRPGLTRKPYQPYAPARDFAAYRP
Mouse CD28 ICD
(SEQ ID NO: 69)
AATAGTAGAAGGAACAGACTCCTTCAAAGTGACTACATGAACATGACTCCCCGGAGGCCTGGGC
TCACTCGAAAGCCTTACCAGCCCTACGCCCCTGCCAGAGACTTTGCAGCGTACCGCCCC
Mouse CD3z
(SEQ ID NO: 70)
KFSRSAETAANLQDPNQLYNELNLGRREEYDVLEKKRARDPEMGGKQQRRRNPQEGVYNALQKD
KMAEAYSEIGTKGERRRGKGHDGLYQGLSTATKDTYDALHMQTLAPR
Mouse CD3z
(SEQ ID NO: 71)
AAGTTCTCAAGATCAGCCGAGACCGCCGCCAACCTGCAGGACCCCAACCAGCTGTACAACGAGC
TGAACCTGGGCAGAAGAGAGGAGTACGACGTGCTGGAGAAGAAGAGAGCCAGAGACCCCGAGAT
GGGCGGCAAGCAGCAGAGAAGAAGAAACCCCCAGGAGGGCGTGTACAACGCCCTGCAGAAGGAC
AAGATGGCCGAGGCCTACTCAGAGATCGGCACCAAGGGCGAGAGAAGAAGAGGCAAGGGCCACG
ACGGCCTGTACCAGGGCCTGTCAACCGCCACCAAGGACACCTACGACGCCCTGCACATGCAGAC
CCTGGCCCCCAGA
Human CD8 leader
(SEQ ID NO: 72)
MALPVTALLLPLALLLHAARP
Human CD8 leader
(SEQ ID NO: 73)
ATGGCCTTACCAGTGACCGCCTTGCTCCTGCCGCTGGCCTTGCTGCTCCACGCCGCCAGGCCG
Human CD19 scFv
(SEQ ID NO: 74)
EIVMTQSPATLSLSPGERATLSCRASQDISKYLNWYQQKPGQAPRLLIYHTSRLHSGIPARFSG
SGSGTDYTLTISSLQPEDFAVYFCQQGNTLPYTFGQGTKLEIKGGGGSGGGGSGGGGSQVQLQE
SGPGLVKPSETLSLTCTVSGVSLPDYGVSWIRQPPGKGLEWIGVIWGSETTYYQSSLKSRVTIS
KDNSKNQVSLKLSSVTAADTAVYYCAKHYYYGGSYAMDYWGQGTLVTVSS
Human CD19 scFv
(SEQ ID NO: 75)
GAAATTGTGATGACCCAGTCACCCGCCACTCTTAGCCTTTCACCCGGTGAGCGCGCAACCCTGT
CTTGCAGAGCCTCCCAAGACATCTCAAAATACCTTAATTGGTATCAACAGAAGCCCGGACAGGC
TCCTCGCCTTCTGATCTACCACACCAGCCGGCTCCATTCTGGAATCCCTGCCAGGTTCAGCGGT
AGCGGATCTGGGACCGACTACACCCTCACTATCAGCTCACTGCAGCCAGAGGACTTCGCTGTCT
ATTTCTGTCAGCAAGGGAACACCCTGCCCTACACCTTTGGACAGGGCACCAAGCTCGAGATTAA
AGGTGGAGGTGGCAGCGGAGGAGGTGGGTCCGGCGGTGGAGGAAGCCAGGTCCAACTCCAAGAA
AGCGGACCGGGTCTTGTGAAGCCATCAGAAACTCTTTCACTGACTTGTACTGTGAGCGGAGTGT
CTCTCCCCGATTACGGGGTGTCTTGGATCAGACAGCCACCGGGGAAGGGTCTGGAATGGATTGG
AGTGATTTGGGGCTCTGAGACTACTTACTACCAATCATCCCTCAAGTCACGCGTCACCATCTCA
AAGGACAACTCTAAGAATCAGGTGTCACTGAAACTGTCATCTGTGACCGCAGCCGACACCGCCG
TGTACTATTGCGCTAAGCATTACTATTATGGCGGGAGCTACGCAATGGATTACTGGGGACAGGG
TACTCTGGTCACCGTGTCCAGC
Human CD8 Hinge
(SEQ ID NO: 76)
TTTPAPRPPTPAPTIASQPLSLRPEACRPAAGGAVHTRGLDFACD
Human CD8 Hinge
(SEQ ID NO: 77)
ACCACGACGCCAGCGCCGCGACCACCAACACCGGCGCCCACCATCGCGTCGCAGCCCCTGTCCC
TGCGCCCAGAGGCGTGCCGGCCAGCGGCGGGGGGCGCAGTGCACACGAGGGGGCTGGACTTCGC
CTGTGAT
Human CD28 TM
(SEQ ID NO: 78)
FWVLVVVGGVLACYSLLVTVAFIIFWV
Human CD28 TM
(SEQ ID NO: 79)
TTTTGGGTGCTGGTGGTGGTTGGTGGAGTCCTGGCTTGCTATAGCTTGCTAGTAACAGTGGCCT
TTATTATTTTCTGGGTG
Human CD28 ICD
(SEQ ID NO: 80)
RSKRSRLLHSDYMNMTPRRPGPTRKHYQPYAPPRDFAAYRS
Human CD28 ICD
(SEQ ID NO: 81)
AGGAGTAAGAGGAGCAGGCTCCTGCACAGTGACTACATGAACATGACTCCCCGCCGCCCCGGGC
CCACCCGCAAGCATTACCAGCCCTATGCCCCACCACGCGACTTCGCAGCCTATCGCTCC
Human CD3z ICD
(SEQ ID NO: 82)
RVKFSRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
Human CD37 ICD
(SEQ ID NO: 83)
AGAGTGAAGTTCAGCAGGAGCGCAGACGCCCCCGCGTACCAGCAGGGCCAGAACCAGCTCTATA
ACGAGCTCAATCTAGGACGAAGAGAGGAGTACGATGTTTTGGACAAGAGACGTGGCCGGGACCC
TGAGATGGGGGGAAAGCCGAGAAGGAAGAACCCTCAGGAAGGCCTGTACAATGAACTGCAGAAA
GATAAGATGGCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTTCACATGCA
GGCCCTGCCCCCTCGC
Mouse CD8 Leader
(SEQ ID NO: 84)
MASPLTRFLSLNLLLLGESIILGSGEA
Mouse CD8 Leader
(SEQ ID NO: 85)
ATGGCCAGCCCCCTGACCAGATTCCTGAGCCTGAACCTGCTGCTGCTGGGCGAGAGCATCATCC
TGGGCAGCGGCGAGGCC
A03 mouse anti-Meso scFv
(SEQ ID NO: 86)
AQVQLQESGPGLVKPSQTLSLTCTVSGGSISSGGYYWSWIRQHPGKGLEWIGYIYYSGSTYYNP
SLKSRVTISVDTSKNQFSLKLSSVTAADTAVYYCARFDYGDFYDAFDIWGQGTMVTVSSGGGGS
GGGGSGGGGSEIVLTQSPSSLSASVGDRVTITCRASQGISSALAWYQQKPGKAPKLLIYDASSL
ESGVPSRFSGSGSGTDFTLTISSLQPEDFATYYCQQFNSYPITFGQGTRLEIKR
A03 mouse anti-Meso scFv
(SEQ ID NO: 87)
GCCCAGGTGCAGCTGCAGGAGTCGGGCCCAGGACTGGTGAAGCCTTCACAGACCCTGTCCCTCA
CCTGCACTGTCTCTGGTGGCTCCATCAGCAGTGGTGGTTACTACTGGAGCTGGATCCGCCAGCA
CCCAGGGAAGGGCCTGGAGTGGATTGGGTACATCTATTACAGTGGGAGCACCTACTACAACCCG
TCCCTCAAGAGTCGAGTTACCATATCAGTAGACACGTCCAAGAACCAGTTCTCCCTGAAGCTGA
GCTCTGTGACCGCTGCGGACACGGCCGTGTATTACTGTGCGAGATTTGACTACGGTGACTTCTA
TGATGCTTTTGATATCTGGGGCCAAGGGACAATGGTCACCGTCTCTTCAGGTGGTGGTGGTAGC
GGCGGCGGCGGCTCTGGTGGTGGTGGATCCGAAATTGTGTTGACGCAGTCTCCATCCTCCCTGT
CTGCATCTGTAGGAGACAGAGTCACCATCACTTGCCGGGCAAGTCAGGGCATTAGCAGTGCTTT
AGCCTGGTATCAGCAGAAACCAGGGAAAGCTCCTAAGCTCCTGATCTATGATGCCTCCAGTTTG
GAAAGTGGGGTCCCATCAAGGTTCAGCGGCAGTGGATCTGGGACAGATTTCACTCTCACCATCA
GCAGCCTGCAGCCTGAAGATTTTGCAACTTATTACTGTCAACAGTTTAATAGTTACCCGATCAC
CTTCGGCCAAGGGACACGACTGGAGATTAAACGT
Mouse CD8 Hinge
(SEQ ID NO: 88)
LQKVNSTTTKPVLRTPSPVHPTGTSQPQRPEDCRPRGSVKGTGLDFACDIY
Mouse CD8 Hinge
(SEQ ID NO: 89)
CTGCAGAAGGTGAACAGCACCACCACCAAGCCCGTGCTGAGAACCCCCAGCCCCGTGCACCCCA
CCGGCACCAGCCAGCCCCAGAGACCCGAGGACTGCAGACCCAGAGGCAGCGTGAAGGGCACCGG
CCTGGACTTCGCCTGCGACATCTAC
Mouse CD8 TM
(SEQ ID NO: 90)
IWAPLAGICVALLLSLIITLI
Mouse CD8 TM
(SEQ ID NO: 91)
ATCTGGGCCCCCCTGGCCGGCATCTGCGTGGCCCTGCTGCTGAGCCTGATCATCACCCTGATC
Mouse 4-1BB ICD
(SEQ ID NO: 92)
KWIRKKFPHIFKQPFKKTTGAAQEEDACSCRCPQEEEGGGGGYEL
Mouse 4-1BB ICD
(SEQ ID NO: 93)
AAGTGGATCAGAAAGAAGTTCCCCCACATCTTCAAGCAGCCCTTCAAGAAGACCACCGGCGCCG
CCCAGGAGGAGGACGCCTGCAGCTGCAGATGCCCCCAGGAGGAGGAGGGCGGCGGCGGCGGCTA
CGAGCTG
Mouse CD3z ICD
(SEQ ID NO: 94)
KFSRSAETAANLQDPNQLYNELNLGRREEYDVLEKKRARDPEMGGKQQRRRNPQEGVYNALQKD
KMAEAYSEIGTKGERRRGKGHDGLYQGLSTATKDTYDALHMQTLAPR
Mouse CD3z ICD
(SEQ ID NO: 95)
AAGTTCAGCAGAAGCGCCGAGACCGCCGCCAACCTGCAGGACCCCAACCAGCTGTACAACGAGC
TGAACCTGGGCAGAAGAGAGGAGTACGACGTGCTGGAGAAGAAGAGAGCCAGAGACCCCGAGAT
GGGCGGCAAGCAGCAGAGAAGAAGAAACCCCCAGGAGGGCGTGTACAACGCCCTGCAGAAGGAC
AAGATGGCCGAGGCCTACAGCGAGATCGGCACCAAGGGCGAGAGAAGAAGAGGCAAGGGCCACG
ACGGCCTGTACCAGGGCCTGAGCACCGCCACCAAGGACACCTACGACGCCCTGCACATGCAGAC
CCTGGCCCCCAGA
Human CD8 Leader
(SEQ ID NO: 96)
MALPVTALLLPLALLLHAARP
Human CD8 Leader
(SEQ ID NO: 97)
ATGGCCCTCCCTGTCACCGCCCTGCTGCTTCCGCTGGCTCTTCTGCTCCACGCCGCTCGGCCC
Human M5 scFv
(SEQ ID NO: 98)
QVQLVQSGAEVEKPGASVKVSCKASGYTFTDYYMHWVRQAPGQGLEWMGWINPNSGGTNYAQKF
QGRVTMTRDTSISTAYMELSRLRSDDTAVYYCASGWDFDYWGQGTLVTVSSGGGGSGGGGSGGG
GSGGGGSDIVMTQSPSSLSASVGDRVTITCRASQSIRYYLSWYQQKPGKAPKLLIYTASILQNG
VPSRFSGSGSGTDFTLTISSLQPEDFATYYCLQTYTTPDFGPGTKVEIK
Human M5 scFv
(SEQ ID NO: 99)
CAAGTCCAACTCGTTCAATCAGGCGCAGAAGTCGAAAAGCCCGGAGCATCAGTCAAAGTCTCTT
GCAAGGCTTCCGGCTACACCTTCACGGACTACTACATGCACTGGGTGCGCCAGGCTCCAGGCCA
GGGACTGGAGTGGATGGGATGGATCAACCCGAATTCCGGGGGAACTAACTACGCCCAGAAGTTT
CAGGGCCGGGTGACTATGACTCGCGATACCTCGATCTCGACTGCGTACATGGAGCTCAGCCGCC
TCCGGTCGGACGATACCGCCGTGTACTATTGTGCGTCGGGATGGGACTTCGACTACTGGGGGCA
GGGCACTCTGGTCACTGTGTCAAGCGGAGGAGGTGGATCAGGTGGAGGTGGAAGCGGGGGAGGA
GGTTCCGGCGGCGGAGGATCAGATATCGTGATGACGCAATCGCCTTCCTCGTTGTCCGCATCCG
TGGGAGACAGGGTGACCATTACTTGCAGAGCGTCCCAGTCCATTCGGTACTACCTGTCGTGGTA
CCAGCAGAAGCCGGGGAAAGCCCCAAAACTGCTTATCTATACTGCCTCGATCCTCCAAAACGGC
GTGCCATCAAGATTCAGCGGTTCGGGCAGCGGGACCGACTTTACCCTGACTATCAGCAGCCTGC
AGCCGGAAGATTTCGCCACGTACTACTGCCTGCAAACCTACACCACCCCGGACTTCGGACCTGG
AACCAAGGTGGAGATCAAG
Human CD8 Hinge
(SEQ ID NO: 100)
TTTPAPRPPTPAPTIASQPLSLRPEACRPAAGGAVHTRGLDFACD
Human CD8 Hinge
(SEQ ID NO: 101)
ACCACTACCCCAGCACCGAGGCCACCCACCCCGGCTCCTACCATCGCCTCCCAGCCTCTGTCCC
TGCGTCCGGAGGCATGTAGACCCGCAGCTGGTGGGGCCGTGCATACCCGGGGTCTTGACTTCGC
CTGCGAT
Human CD8 TM
(SEQ ID NO: 102)
IYIWAPLAGTCGVLLLSLVITLYC
Human CD8 TM
(SEQ ID NO: 103)
ATCTACATTTGGGCCCCTCTGGCTGGTACTTGCGGGGTCCTGCTGCTTTCACTCGTGATCACTC
TTTACTGT
Human 4-1BB ICD
(SEQ ID NO: 104)
KRGRKKLLYIFKQPFMRPVQTTQEEDGCSCRFPEEEEGGCEL
Human 4-1BB ICD
(SEQ ID NO: 105)
AAGCGCGGTCGGAAGAAGCTGCTGTACATCTTTAAGCAACCCTTCATGAGGCCTGTGCAGACTA
CTCAAGAGGAGGACGGCTGTTCATGCCGGTTCCCAGAGGAGGAGGAAGGCGGCTGCGAACTG
Human CD3z ICD
(SEQ ID NO: 106)
RVKFSRSADAPAYKQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
Human CD3z ICD
(SEQ ID NO: 107)
CGCGTGAAATTCAGCCGCAGCGCAGATGCTCCAGCCTACAAGCAGGGGCAGAACCAGCTCTACA
ACGAACTCAATCTTGGTCGGAGAGAGGAGTACGACGTGCTGGACAAGCGGAGAGGACGGGACCC
AGAAATGGGCGGGAAGCCGCGCAGAAAGAATCCCCAAGAGGGCCTGTACAACGAGCTCCAAAAG
GATAAGATGGCAGAAGCCTATAGCGAGATTGGTATGAAAGGGGAACGCAGAAGAGGCAAAGGCC
ACGACGGACTGTACCAGGGACTCAGCACCGCCACCAAGGACACCTATGACGCTCTTCACATGCA
GGCCCTGCCGCCTCGG

### E. T Cell Receptors

In some aspects, the invention provides a method of producing a modified immune cell (e.g., T cell) responsive to orthogonal cytokine signaling, in which the modified immune cell is an immune effector cell which has been modified to express a T cell receptor (TCR). In some aspects, the invention provides a modified immune cell (e.g., T cell) responsive to orthogonal cytokine signaling, in which the modified immune cell is an immune effector cell which has been modified to express a T cell receptor (TCR).

In some embodiments, the TCR targets (i.e., has antigenic specificity for) an antigen, for example, a tumor antigen. As used herein, the phrase “having antigenic specificity,” or like phrase, means that the TCR can specifically bind to and recognize the antigen, or an epitope thereof.

Natural TCRs are generally hetero dimers. In humans, in 95% of T cells, the TCR comprises an alpha (α) chain and a beta (β) chain (encoded by TRA and TRB, respectively), whereas in 5% of T cells, the TCR comprises gamma and delta (γ/δ) chains (encoded by TRG and TRD, respectively). Natural TCR complexes are an octameric assembly of type-I single-spanning membrane proteins arranged into four dimeric modules: the variable ligand-binding TCRαβ module (in most T cells) (or the TCR γ/δ module) and the three invariant signaling modules CD3δε, CD3γε, and CD3ζ dimer. The TCRαβ module binds to pMHC ligands on APC or target cell surfaces, but these proteins lack intrinsic signaling capability and, as such, rely on the signaling modules to transmit information through their cytoplasmic immunoreceptor tyrosine-based activation motifs (ITAMs). See, e.g., Chandler et al. Int J Mol Sci (2020) 21:7424, which is incorporated by reference herein. Natural TCRs can be cloned and modified using standard molecular biology and genetic engineering techniques known in the art.

Various engineered TCR forms are also known in the art, including TCR mimic antibodies (Chang et al., Expert Opin Biol Ther. (2016) 16(8):979-87), TCR-like CARs and TCR-CARs (Walseng et al., Sci Rep. (2017) 7:1-10; Akatsuka et al., Front Immunol. (2020) 11:257; Poorebrahim et al., Cancer Gene Ther. (2021) 28(6):581-589. Unlike CARs, TCRs are not restricted to the cell surface antigens, but can detect and bind to the peptides presented by MHC molecules (pMHCs). This feature provides a wide range of potential targets for TCRs such as tumor-specific neoepitopes. Of note, redirection of TCR-based CARs on the highly tumor-specific neoepitopes can prevent “off-tumor” toxicities that are commonly associated with CAR therapies

In some embodiments, the TCR is a natural TCR. In some embodiments, the TCR is a modified TCR. In some embodiments, the TCR is an engineered TCR, such as a TCR mimic or antibody-like structure, a CAR-like TCR, or a CAR-TCR. In some embodiments, the TCR is a murine TCR. In some embodiments, the TCR is a human TCR. In some embodiments, the TCR is a hybrid TCR having one or more portions of a human TCR (e.g., a constant portion or a variable portion) and one or more portions of a murine TCR (e.g., a constant portion or a variable portion). Alternatively, the portion can be a few amino acids of a human TCR, such that the TCR, which is mostly murine, is “humanized.” Methods of making such hybrid TCRs are known in the art (see, for example, Cohen et al., Cancer Res., (2006) 66:8878-8886).

In some embodiments, the TCR targets (i.e., has antigenic specificity for) a gp100 melanoma antigen, e.g., human gp100. In some embodiments, the tumor antigen is gp100. gp100, also known in the art as SILV, SI, SIL, ME20, PMEL17, or D12S53E. gp100, is a protein known to play an important role in regulating mammalian pigmentation (Hoashi et al., J. Biol. Chem. (2005) 280:14006-14016) and is known as a cancer antigen expressed by human tumors, including melanoma and colorectal tumors (Tartaglia et al., Vaccine (2001) 19(17-19):2571-5). The amino acid and nucleotide sequence es of human gp100 are published in the GenBank database of the National Center for Biotechnology Information (NCBI) as GenBank Accession No. NP_008859 (amino acid sequence) and GenBank Accession No. NM_006928.3 (nucleotide sequence).

TCRs having antigenic specificity for gp100 (i.e., anti-gp100 TCRs) are known in the art, such as the TCRs described in, e.g., US20140219978A1. In some embodiments, the TCR is a pmel-1 TCR. The pmel-1 mouse model was developed as a system to model treatment of malignant melanoma using adoptive cell therapy (ACT) (Overwijk, et al., J Exp Med. (2003), 198(4):569-80). The target antigen, pmel-17, is an ortholog of the melanocyte differentiation antigen gp100, which is often overexpressed in human melanomas.

In some embodiments, the TCR targets (i.e., has antigenic specificity for) an NY-ESO-1 antigen. NY-ESO-1 is a cancer-testis antigen overexpressed in synovial sarcoma, myxoid liposarcoma, melanoma and other tumors. In some embodiments, the TCR is an NYESO1-TCR clone 1G4 (Robbins, et al., J Immunol, 2008, 180:6116-6131). In some embodiments, the TCR targets an antigen selected from MAGE-A3/A6, MAGE-A10, AFP, PRAME, MART-1, and HPV E6.

### F. Nucleic Acids and Expression Vectors

The present disclosure provides nucleic acids encoding an orthogonal cytokine receptor, an orthogonal cytokine, a CAR, and/or a TCR. The nucleic acid of the present disclosure may comprises a nucleotide sequence (i.e., a polynucleotide sequence) encoding any one of the orthogonal cytokine receptors, orthogonal cytokines, CARs, and/or TCRs disclosed herein. In certain embodiments, the modified immune cell comprises an exogenous nucleic acid comprising a nucleotide sequence encoding the oIL2Rβ. In some embodiments, the modified immune cell comprises a vector expressing the oIL2Rβ. In some embodiments, the vector is a viral vector selected from an adenoviral vector, an adeno-associated virus (AAV) vector, a lentiviral vector, and a retroviral vector.

In certain embodiments, a nucleic acid of the present disclosure comprises a first polynucleotide sequence and a second polynucleotide sequence. The first and second polynucleotide sequence may be separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. In certain embodiments, the nucleic acid comprises from 5′ to 3′ the first polynucleotide sequence the linker, and the second polynucleotide sequence. In certain embodiments, the nucleic acid comprises from 5′ to 3′ the second polynucleotide sequence the linker, and the first polynucleotide sequence e.

In some embodiments, the linker comprises a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in the present invention.

In some embodiments, the linker comprises a nucleic acid sequence that encodes for a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide for use in the present invention.

In some embodiments, a linker further comprises a nucleic acid sequence that encodes a furin cleavage site. Furin is a ubiquitously expressed protease that resides in the trans-golgi and processes protein precursors before their secretion. Furin cleaves at the COOH— terminus of its consensus recognition sequence Various furin consensus recognition sequence es (or “furin cleavage sites”) are known to those of skill in the art, including, without limitation, Arg-X1-Lys-Arg or Arg-X1-Arg-Arg, X2-Arg-X1-X3-Arg, and Arg-X1-X1-Arg, such as an Arg-Gln-Lys-Arg, where X1 is any naturally occurring amino acid, X2 is Lys or Arg, and X3 is Lys or Arg. Those of skill in the art would be able to select the appropriate Furin cleavage site for use in the present invention.

In some embodiments, the linker comprises a nucleic acid sequence encoding a combination of a Furin cleavage site and a 2A peptide. Examples include, without limitation, a linker comprising a nucleic acid sequence encoding a Furin cleavage site and F2A, a linker comprising a nucleic acid sequence encoding a Furin cleavage site and E2A, a linker comprising a nucleic acid sequence encoding a Furin cleavage site and P2A, a linker comprising a nucleic acid sequence encoding a Furin cleavage site and T2A. Those of skill in the art would be able to select the appropriate combination for use in the present invention. In such embodiments, the linker may further comprise a spacer sequence between the Furin cleavage site and the 2A peptide. In some embodiments, the linker comprises a Furin cleavage site 5′ to a 2A peptide. In some embodiments, the linker comprises a 2A peptide 5′ to a Furin cleavage site. Various spacer sequence es are known in the art, including, without limitation, glycine serine (GS) spacers (also known as GS linkers) such as (GS)n, (SG)n, (GSGGS)n and (GGGS)n, where n represents an integer of at least 1. Exemplary spacer sequence es can comprise amino acid sequence es including, without limitation, GGSG, GGSGG, GSGSG, GSGGG, GGGSG, GSSSG, and the like. Those of skill in the art would be able to select the appropriate spacer sequence for use in the present invention.

In some embodiments, a nucleic acid of the present disclosure may be operably linked to a transcriptional control element, e.g., a promoter, and enhancer, etc. Suitable promoter and enhancer elements are known to those of skill in the art.

For expression in a bacterial cell, suitable promoters include, but are not limited to, lad, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Lad repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence e capable of driving high levels of expression of any polynucleotide sequence operatively linked thereto. Other constitutive promoter sequence es may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is for the production of a transgenic polypeptide product that is released upon CAR signaling. See, e.g., Chmielewski and Abken, Expert Opin. Biol. Ther. (2015) 15(8): 1145-1154; and Abken, Immunotherapy (2015) 7(5): 535-544. In some embodiments, a nucleic acid of the present disclosure further comprises a nucleic acid sequence e encoding a cytokine operably linked to a T-cell activation responsive promoter. In some embodiments, the cytokine operably linked to a T-cell activation responsive promoter is present on a separate nucleic acid sequence. In one embodiment, the cytokine is IL-12.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequence es encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the CAR or TCR into an immune cell or precursor thereof (e.g., a T cell). Accordingly, an expression vector (e.g., a lentiviral vector) of the present invention may comprise a nucleic acid encoding for a CAR or a TCR. In some embodiments, the expression vector (e.g., lentiviral vector) will comprise additional elements that will aid in the functional expression of the CAR or TCR encoded therein. In some embodiments, an expression vector comprising a nucleic acid encoding for a CAR or TCR further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR- or TCR-encoding nucleic acid sequence). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequence es are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence Various posttranscriptional regulator elements are known to those of skill in the art and may be incorporated into a vector (e.g., lentiviral vector) of the present invention. A vector of the present invention may further comprise additional elements such as a rev response element (RRE) for RNA transport, packaging sequence es, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence cPPT sequence RRE sequence 5′LTR, 3′ U3 deleted LTR′ in addition to a nucleic acid encoding for a CAR or a TCR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding an orthogonal cytokine receptor or its corresponding orthogonal cytokine, and/or a CAR or TCR of the present disclosure. Methods for introducing RNA into a host cell are known in the art. See, e.g., Zhao et al. Cancer Res. (2010) 15: 9053. Introducing RNA comprising a nucleotide sequence encoding a CAR or TCR of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence e encoding an orthogonal cytokine receptor or its corresponding orthogonal cytokine, and/or a CAR or TCR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequence es to enable expression in the host cells. Useful selectable markers include, without limitation, antibiotic-resistance genes.

Reporter genes are used for identifying potentially transfected cells and for evaluating the functionality of regulatory sequence es. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present disclosure is provided for the production of (i) an orthogonal cytokine receptor, (ii) an orthogonal cytokine, and/or (iii) a CAR or TCR as described herein, e.g., in a mammalian cell. In some embodiments, a nucleic acid of the present disclosure provides for amplification of the nucleic acid.

Oncolytic Adenoviral Vectors

Oncolytic viruses represent highly promising agents for the treatment of solid tumors, and an oncolytic herpes virus expressing GM-CSF was approved by the US FDA for the therapy of advanced melanoma based on therapeutic benefit demonstrated in a clinical study (Andtbacka R H, et al. Talimogene laherparepvec improves durable response rate in patients with advanced melanoma. J Clin Oncol. 2015; 33(25):2780-2788). Oncolytic adenoviruses (OAds) can be programmed to specifically target, replicate in, and kill cancer cells while sparing normal cells. The release of virus progeny results in an exponential increase of the virus inoculum, which can cause direct tumor debulking while providing danger signals necessary to awaken the immune system (Lichty B D, Breitbach C J, Stojdl D F, Bell J C. Going viral with cancer immunotherapy. Nat Rev Cancer. 2014; 14(8):559-567). Importantly, OAds can be genetically modified to express therapeutic transgenes selectively in the TME (Siurala M, et al. Adenoviral delivery of tumor necrosis factor-α and interleukin-2 enables successful adoptive cell therapy of immunosuppressive melanoma. Mol Ther. 2016; 24(8):1435-1443; Nishio N, et al. Armed oncolytic virus enhances immune functions of chimeric antigen receptor-modified T cells in solid tumors. Cancer Res. 2014; 74(18):5195-5205; Tanoue K, et al. Armed oncolytic adenovirus-expressing PD-L1 mini-body enhances antitumor effects of chimeric antigen receptor T cells in solid tumors. Cancer Res. 2017; 77(8):2040-2051; Rosewell Shaw A, et al. Adenovirotherapy delivering cytokine and checkpoint inhibitor augments CAR T cells against metastatic head and neck cancer. Mol Ther. 2017; 25(11):2440-2451). The feasibility and safety of OAds in human patients have been demonstrated in clinical trials (Kim K H, et al. A phase I clinical trial of Ad5/3-Δ24, a novel serotype-chimeric, infectivity-enhanced, conditionally-replicative adenovirus (CRAd), in patients with recurrent ovarian cancer. Gynecol Oncol. 2013; 130(3):518-524; Ranki T, et al. Phase I study with ONCOS-102 for the treatment of solid tumors—an evaluation of clinical response and exploratory analyses of immune markers. J Immunother Cancer. 2016; 4:17). Their ability to revert tumor immunosuppression while locally expressing therapeutic transgenes provides a rational strategy for combination with adoptive T cell transfer.

In some embodiments, an orthogonal cytokine of the present disclosure, e.g., orthogonal IL-2, is encoded by a nucleic acid sequence which is comprised within an oncolytic adenoviral vector such as a conditionally replicating oncolytic adenoviral vector. One example of a conditionally replicating oncolytic adenoviral vector includes a serotype 5 adenoviral vector (Ad5) with modifications to the early genes E1A and E3 to enable cancer cell—specific replication and transgene expression, respectively. E1A is modified by deleting 24 base pairs of DNA from the CR2 region (aka D24 variant) to yield a virus capable of selectively replicating in cancer cells harboring p16-Rb pathway mutations. The orthogonal cytokine (e.g., oIL2) transgene may be placed in the E3 region. Furthermore, the virus capsid is modified to include a chimeric 5/3 fiber which enables improved transduction efficiency of tumor cells. This construct, Ad5/3-D24-orthoIL2, and its isogenic controls are used in in vitro and in vivo studies to assess the ability of orthogonal cytokine pairs to selectively attract and stimulate lentivirally transduced orthoCAR T cells and/or orthoTCR T cells for improved antitumor efficacy.

### G. Methods of Treatment

The modified cells (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the invention includes a method for adoptive cell transfer therapy comprising administering to a subject in need thereof a modified immune cell of the present invention. In another aspect, the invention includes a method of treating a disease or condition in a subject comprising administering to a subject in need thereof a population of modified immune cells. In one aspect, the invention provides a method of treating cancer comprising administering an effective amount of a modified immune cell disclosed herein and an effective amount of an orthogonal ctyokine to a subject having cancer. In one aspect, the invention provides a method of treating cancer comprising administering an effective amount of a modified immune cell described herein and an effective amount of an orthogonal cytokine or a vector expressing the orthogonal cytokine.

In one aspect, the invention provides a method of treating cancer in a subject, the method comprising (a) administering to the subject an effective amount of the modified immune cell responsive to orthogonal cytokine signaling disclosed herein, and (b) administering to the subject an effective amount of an orthogonal interleukin-2 (oIL2) which binds to the oIL2Rβ, or a vector which expresses the oIL2. In certain embodiments, the vector which expresses oIL2 is a viral vector. In some embodiments, the viral vector is selected from an adenoviral vector, an adeno-associated virus (AAV) vector, a lentiviral vector, and a retroviral vector. In some embodiments, administering comprises intravenous administration and/or intratumoral injection. In some embodiments, the immune effector cell is a human cell and the subject is a human. In some embodiments, the immune effector cell is a human T cell and the subject is a human. In some embodiments, the method further comprises discontinuing administration of the oIL2 or the vector which expresses the oIL2.

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a solid tumor or a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

In certain exemplary embodiments, the modified immune cells of the invention are used to treat a myeloma, or a condition related to myeloma. Examples of myeloma or conditions related thereto include, without limitation, light chain myeloma, non-secretory myeloma, monoclonal gamopathy of undertermined significance (MGUS), plasmacytoma (e.g., solitary, multiple solitary, extramedullary plasmacytoma), amyloidosis, and multiple myeloma. In one embodiment, a method of the present disclosure is used to treat multiple myeloma. In one embodiment, a method of the present disclosure is used to treat refractory myeloma. In one embodiment, a method of the present disclosure is used to treat relapsed myeloma.

In certain exemplary embodiments, the modified immune cells of the invention are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In one embodiment, a method of the present disclosure is used to treat cutaneous melanoma. In one embodiment, a method of the present disclosure is used to treat refractory melanoma. In one embodiment, a method of the present disclosure is used to treat relapsed melanoma.

In yet other exemplary embodiments, the modified immune cells of the invention are used to treat a sarcoma, or a condition related to sarcoma. Examples of sarcoma or conditions related thereto include, without limitation, angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, and synovial sarcoma. In one embodiment, a method of the present disclosure is used to treat synovial sarcoma. In one embodiment, a method of the present disclosure is used to treat liposarcoma such as myxoid/round cell liposarcoma, differentiated/dedifferentiated liposarcoma, and pleomorphic liposarcoma. In one embodiment, a method of the present disclosure is used to treat myxoid/round cell liposarcoma. In one embodiment, a method of the present disclosure is used to treat a refractory sarcoma. In one embodiment, a method of the present disclosure is used to treat a relapsed sarcoma.

The cells of the invention to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, alymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1 ×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1 ×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8⁺ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4⁺ and CD8⁺ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells of the invention may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR or TCR. In certain embodiments, combination treatment comprising an immune checkpoint modulator may increase the therapeutic efficacy of a therapy comprising a modified cell of the present invention.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to adoptive cell therapy (e.g., CAR T cell therapy). In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to adoptive cell therapy (e.g., CAR T cell therapy) may increase the efficacy of the adoptive cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

Adoptive cell therapy providing the modified immune cells described herein has the potential to enhance on-target off-tumor toxicity. As such, it is contemplated herein that the adoptive cell therapy methods (i.e., methods of treating cancer), in certain embodiments, further comprise additional cell engineering strategies (e.g. on/off systems, synthetic circuits) to maximize patient safety (see, e.g., Caliendo, et al., Front Bioeng Biotechnol. (2019) 7:43. Importantly, it is contemplated herein that the modified immune cells of the invention comprise a safety strategy in that the modified immune cells are endogenous IL-2−/− and therefore dependent upon orthogonal IL-2 (oIL2) administration to the subject. As such, methods of treatment comprising administration of the modified immune cells described herein and oIL2 further comprise discontinuing administration of the oIL2 or the vector expressing oIL2. In certain embodiments, the oIL2 administration is discontinued due to a safety concern such as toxicity.

In one aspect, the invention includes a method of treating cancer in a subject in need thereof, comprising administering to the subject any one of the modified immune cells disclosed herein. Yet another aspect of the invention includes a method of treating cancer in a subject in need thereof, comprising administering to the subject a modified immune cell produced by any one of the methods disclosed herein.

### H. Sources of Immune Cells

The modified immune cells of the present invention are derived from immune effector cells that are responsive to interleukin-2 (IL-2) and interleukin-15 (IL-15). In certain embodiments, a source of immune cells (e.g. T cells) is obtained from a subject for ex vivo manipulation. Sources of target cells for ex vivo manipulation may also include, e.g., autologous or heterologous donor blood, cord blood, or bone marrow. For example the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. n yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### I. Expansion of Immune Cells

Whether prior to or after modification of cells to express a CAR or TCR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### J. Pharmaceutical Compositions and Formulations

Also provided are populations of immune cells of the invention, compositions containing such cells and/or enriched for such cells, such as in which the modified immune cells make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Orthogonal Cytokine Enhanced CAR T Cells Generated Through Gene Editing

Materials and Methods

Cell Culture Conditions and Expansion of Primary Human T Lymphocytes

SeAx, an IL-2—dependent cutaneous T-cell lymphoma (CTCL) cell line, was cultured in RPMI 1640 (Gibco) supplemented with 10% FBS (Sigma-Aldrich), 1% 1-glutamine (Gibco), 1% Hepes (Gibco), 1% pen/strep (Gibco), and recombinant human IL-2 (5 ng/ml; R&D Systems). Primary human T cells were ordered from Human Immunology Core of the University of Pennsylvania and cultured in X-Vivo15 medium (Lonza) with 10% Fetal Bovine Serum, 50 uM 2-mercaptoethanol, and 10 mM N-Acetyl L-Cystine. T cells were stimulated for 2 days with anti-human CD3/CD28 magnetic dynabeads (ThermoFisher) at a beads to cells concentration of 3:1, along with a cytokine cocktail of IL-2 at 200 U/mL (UPenn hospital pharmacy), IL-7 and IL15 at ng/mL (ThermoFisher). All cells were grown in 5% CO₂, 95% air-humidified incubator at 37° C.

Prime Editing

PE2 mRNA

The Cas9 nickase-reverse transcriptase plasmid (pCMV-PE2, Addgene #132775) was linearized with NruI (New England Biolabs #R0192S) for overnight at 37° C. and purified with QIAquick PCR cation kit (Qiagen #28104). Linearized and purified pCMV-PE2 was then in vitro transcribed using T7 mScript Standard mRNA Production System (Cell Script #C-MSC11610) and purified with RNeasy Mini Kit (Qiagen #474104) according to the manufacturer's instructions. PE2 mRNA was dissolved in RNAse-free water and kept in 80° C.

PegRNA

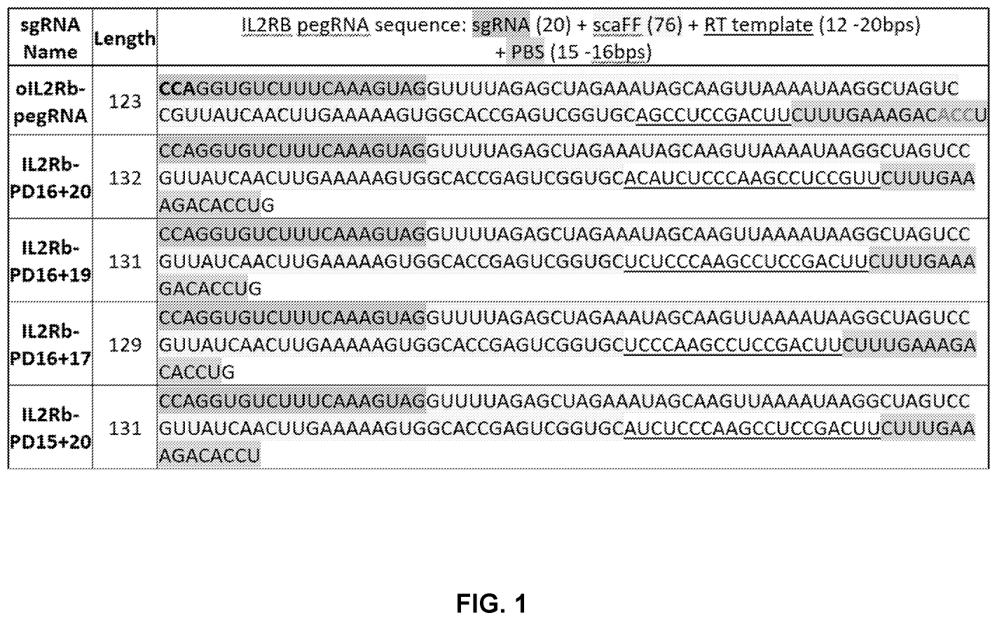

Five pegRNAs (SEQ ID NOs: 1, 6, 11, 16, and 21) were designed using PegFinder (http://pegfinder.sidichenlab.org/) and synthesized by Agilent (FIG. 1). Based on the original prime editing report, the reverse transcriptase (RT) templates of 12-20 nucleotides in length, inclusive of the C>G transversion, and the primer binding sites (PBS) of 15-16 nucleotides in length were selected as shown in FIG. 1.

Electroporation of PE-pegRNA

SeAx or T cells were electroporated using Amaxa P3 Primary Cell 4D-Nucleofector #V4XP-3032 or V4XP-3024 (Lonza) according to the manufacturer's protocol with 1×10⁶ or 5×10⁶ cells (program E0115), PE mRNA, pegRNA, and ngRNA (1:1:1). Following electroporation, cells were rescued with prewarmed growth media and incubated for at least 15 minutes. Cells were then transferred to fresh plates or flasks and diluted to 1.0×10⁶ cells/ml in growth medium as described above. Fresh oIL2 and media were added every 2-3 days.

Genomic DNA Extraction, PCR, and Sequencing

Genomic DNA was extracted using DNeasy Blood & Tissue Kit (Qiagen) following manufacturer's instructions. 100 ng gDNA was used for PCR with primers (Table 1) using Q5 Hot Start High-Fidelity 2× Master Mix (New England Biolabs). 5 μl of PCR product was run on an agarose gel to check for the correct amplification of DNA fragment and the rest of PCR product was purified for sequencing with primers (Table 1) (Genewiz).

TABLE 1
Primers

| Primer | Sequence |
|---|---|
| Fwd-596 (peg) primer for PCR | GCAGAGTGGTGAGTGGTCAG |
| (SEQ ID NO: 151) | |
| Rev-596 (peg) primer for PCR | TCTGAATCTTTCCCTGGTGT |
| (SEQ ID NO: 152) | C |
| Fwd-322 (peg) primer for | GTAGGGGAGGTGGTAGCATG |
| sequencing (SEQ ID NO: 153) | |
| Rev-322 (peg) primer for | AAAGGGACAGGACATGGACC |
| sequencing (SEQ ID NO: 154) | |
| LHA 5′-20 (long) (SEQ ID NO: | TTTGTATCCCCACCCCCTTA |
| 155) | |
| LHA 3′-24 (long) (SEQ ID NO: | ATGAGCTGCTATTAGTCCCA |
| 156) | TCTG |
| LHA 5′-20 (short) (SEQ ID | CAGTCAGTCTTTGGGGGTTT |
| NO: 157) | |
| LHA 3′-21 (short) (SEQ ID | CTGGTGAGTTTGGGATTCTT |
| NO: 158) | G |
| LHA-Fwd-66 (SEQ ID NO: 159) | TCTTGTTCAAGAGTTCCCTA |
| | TCAC |
| RHA-Rev-71 (SEQ ID NO: 160) | TGAAGTAGGTGCACTGTTTG |
| | T |
| LHA-Fwd-92 (SEQ ID NO: 161) | CCAGAATTAACAGTATAAAT |
| | TGCATC |
| RHA-Rev-97 (SEQ ID NO: 162) | TGTAGCTGTGTTTTCTTTGT |
| | AGA |
| LHA-Fwd-155 (SEQ ID NO: 163) | CAGGTAAAGTCTTTGAAAAT |
| | ATGTGT |
| RHA-Rev-140 (SEQ ID NO: 164) | TCCATTCAAAATCATCTGTA |
| | AATCCA |
| EF1-IL2-GFP (SEQ ID NO: 165) | GTTAATTGCATGAATTAGAG |
| | CTA |
| EF1-IL2-GFP (SEQ ID NO: 166) | AAAATATTGTACTTACCTTC |
| | TTGG |
| IL2-GFP 5′-547 before arm | CTGTTTACTCTTGCTCTTGT |
| (SEQ ID NO: 167) | CCA |
| IL2-GFP 3′-547 on CD19CAR | GGCAGACAGGGAGGATGTAG |
| (SEQ ID NO: 168) | |
| IL2-GFP 5′-600 after arm | CACTCCCACTGTCCTTTCCT |
| (SEQ ID NO: 169) | |
| IL2-GFP 3′-600 on CD19CAR | AGGCTTCATTATCAAACTTG |
| (SEQ ID NO: 170) | GGT |

CRISPR/Cas9-Mediated CAR Knock-In

Generation of dsDNA or ssDNA Donor Template

The CAR19 with homology arm(s) sequence (Table 2) was synthesized and subcloned into PUC-GW-Amp vector (Genewiz) as a PCR amplification plasmid.

TABLE 2
CAR19 with homology arm(s) sequence
Sequence of CAR19 donor template (SEQ ID NO: 171)
LHA MNDU3_kozak_CD19BBZ_polyA RHA (360 + 715 + 1458 + 225 + 385 = 3143 bp)
TTTGTATCCCCACCCCCTTAAAGAAAGGAGGAAAAACTGTTTCATACAGAAGGCGTTAATTGCATGAATTAGAGCTATCA
CCTAAGTGTGGGCTAATGTAACAAAGAGGGATTTCACCTACATCCATTCAGTCAGTCTTTGGGGGTTTAAAGAAATTCCA
AAGAGTCATCAGAAGAGGAAAAATGAAGGTAATGTTTTTTCAGACAGGTAAAGTCTTTGAAAATATGTGTAATATGTAAA
ACATTTTGACACCCCCATAATATTTTTCCAGAATTAACAGTATAAATTGCATCTCTTGTTCAAGAGTTCCCTATCACTCT
CTTTAATCACTACTCACAGTAACCTCAACTCCTGCAACAAttttaaaagaaaaggggggattggggggtacagtgcaggg
gaaagaatagtagacataatagcaacagacatacaaactaaagaattacaaaaacaaattacaaaaattcaaaattttcg
ggtttattacagggacagcagagatccagtttgggaattagcttgatcgattagtccaatttgttaaagacaggatatca
gtggtccaggctctagttttgactcaacaatatcaccagctgaagcctatagagtacgagccatagatagaataaaagat
tttatttagtctccagaaaaaggggggaatgaaagaccccacctg [symbol] aggtttggcaagctaggatcaaggttaggaacag
agagacagcagaatatgggccaaacaggatatctgtggtaagcagttcctgccccggctcagggccaagaacagttggaa
cagcagaatatgggccaaacaggatatctgtggtaagcagttcctgccccggctcagggccaagaacagatggtccccag
atgcggtcccgccctcagcagtttctagagaaccatcagatgtttccagggtgccccaaggacctgaaatgaccctgtgc
cttatttgaactaaccaatcagttcgcttctcgcttctgttcgcgcgcttctgctccccgagctcaataaaagagcccac
aacccctcactcgg [symbol] gcgatctagatctcGCCACCATGGCCTTACCAGTGGCCTTGCTCCTGCCGCTGGCCTTGCTGCTC
CACGCCGCCAGGCCGGACATCCAGATGACACAGACTACATCCTCCCTGTCTGCCTCTCTGGGAGACAGAGTCACCATCAG
TTGCAGGGCAAGTCAGGACATTAGTAAATATTTAAATTGGTATCAGCAGAAACCAGATGGAACTGTTAAACTCCTGATCT
ACCATACATCAAGATTACACTCAGGAGTCCCATCAAGGTTCAGTGGCAGTGGGTCTGGAACAGATTATTCTCTCACCATT
AGCAACCTGGAGCAAGAAGATATTGCCACTTACTTTTGCCAACAGGGTAATACGCTTCCGTACACGTTCGGAGGGGGGAC
CAAGCTGGAGATCACAGGTGGCGGTGGCTCGGGCGGTGGTGGGTCGGGTGGCGGCGGATCTGAGGTGAAACTGCAGGAGT
CAGGACCTGGCCTGGTGGCGCCCTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGGGGTAGTGAAACCACATACTATAA
TTCAGCTCTCAAATCCAGACTGACCATCATCAAGGACAACTCCAAGAGCCAAGTTTTCTTAAAAATGAACAGTCTGCAAA
CTGATGACACAGCCATTTACTACTGTGCCAAACATTATTACTACGGTGGTAGCTATGCTATGGACTACTGGGGCCAAGGA
ACCTCAGTCACCGTCTCCTCAACCACGACGCCAGCGCCGCGACCACCAACACCGGCGCCCACCATCGCGTCGCAGCCCCT
GTCCCTGCGCCCAGAGGCGTGCCGGCCAGCGGCGGGGGGCGCAGTGCACACGAGGGGGCTGGACTTCGCCTGTGATATCT
ACATCTGGGCGCCCTTGGCCGGGACTTGTGGGGTCCTTCTCCTGTCACTGGTTATCACCCTTTACTGCAAACGGGGCAGA
AAGAAACTCCTGTATATATTCAAACAACCATTTATGAGACCAGTACAAACTACTCAAGAGGAAGATGGCTGTAGCTGCCG
ATTTCCAGAAGAAGAAGAAGGAGGATGTGAACTGAGAGTGAAGTTCAGCAGGAGCGCAGACGCCCCCGCGTACAAGCAGG
GCCAGAACCAGCTCTATAACGAGCTCAATCTAGGACGAAGAGAGGAGTACGATGTTTTGGACAAGAGACGTGGCCGGGAC
CCTGAGATGGGGGGAAAGCCGAGAAGGAAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACGATGGCCTTTACCAGGGTCTCAGTACAG
CCACCAAGGACACCTACGACGCCCTTCACATGCAGGCCCTGCCCCCTCGCTAACTGTGCCTTCTAGTTGCCAGCCATCTG
TTGTTTGCCCCTCCCCCGTGCCTTCCTTGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGCAGGACAGCAAGGGGGAGGATTGG [symbol]
ATAGCAG [symbol] TGGGGATGCGGTGGGCTCTATGGTGTACATGATGCAACTCCTGTCTTGCATTGCACTAAG
TCTTGCACTTGTCACAAACAGTGCACCTACTTCAAGTTCTACAAAGAAAACACAGCTACAACTGGAGCATTTACTGCTGG
ATTTACAGATGATTTTGAATGGAATTAATGTAAGTATATTTCCTTTCTTACTAAAATTATTACATTTAGTAATCTAGCTG
GAGATCATTTCTTAATAACAATGCATTATACTTTCTTAGAATTACAAGAATCCCAAACTCACCAGGATGCTCACATTTAA
GTTTTACATGCCCAAGAAGGTAAGTACAATATTTTATGTTCAATTTCTGTTTTAATAAAATTCAAAGTAATATGAAAATT
TGCACAGATGGGACTAATAGCAGCTCAT
CA19BBZ amino acid sequence (SEQ ID NO: 172)
MALPVALLLPLALLLHAARPDIQMTOTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEITGGGGSGGGGSGGGGSEVKLQESGPGLVAPSQSL
SVTCTVSGVSLPDYGVSWIRQPPRKGLEWLGVIWGSETTYYNSALKSRLTIIKDNSKSQVFLKMNSLQTDDTAIYYCAKH
YYYGGSYAMDYWGQGTSVTVSSTTTPAPRPPTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTATKDTYDALHMQ
ALPPR

The plasmid was transformed and amplified in DH5a bacterial cells and grown overnight. DNA was extracted by Endo Free Plasmid Maxi Kit (Qiagen). Primers were designed using SnapGene™ for different homology arm lengths (100, 200, 300, and 400 bp) for insertion in the IL2 locus (Table 1). The dsDNA was generated by PCR amplification with primers (Table 1) using Q5 Hot Start High-Fidelity DNA Polymerase or Q5 Hot Start High-Fidelity 2X Master Mix (New England Biolabs), forward and reverse primer (1 plasmid DNA (15-20 ng), and nuclease-free water, in a final volume of 100 μL. PCR reactions were run on the VeritiPro PCR System (Applied Biosystems) according to the following program: Initial denaturation at 98° C. for 30 sec, 30-35 cycles of each 3 steps (denaturation at 98° C. for 10 sec, annealing at +3° C. of lower melting temperature of primer for 20 sec, and extension at 72° C. for variable time based on PCR product size—30 sec/kb), final extension at 72° C. for 2 min. PCR reactions were run on 0.8-1% agarose gel for band size confirmation. The 1 kb plus DNA Ladder (Invitrogen) was used in all experiments. PCR products were purified using QIAquick PCR Purification Kit (Qiagen) or the NucleoSpin® Gel and PCR clean up kit (Takara Bio). To generate highly concentrated dsDNA, 20 PCR reactions were combined. For ssDNA generation, PCR reaction was performed with one of primers (forward or reverse) containing a 5′ phosphorylation in order to generate the antisense or sense ssDNA, respectively. The long CAR19 ssDNA was produced using gene Guide-it Long ssDNA Production System v2 according to the manufacturer's protocol. To generate highly concentrated ssDNA, 1/10th the volume of 3 M sodium acetate, pH 5.2, and an equal volume of isopropanol were added into the purified ssDNA and incubated for 15 min on dry ice followed by washing with 80% ethanol.

Generation of Knock-In T Cells

Primary human T cells were ordered from Human Immunology Core of the University of Pennsylvania and cultured in X-Vivo15 medium (Lonza) with 10% Fetal Bovine Serum, 50 2-mercaptoethanol, and 10 mM N-Acetyl L-Cysteine. T cells were stimulated for 2 days with anti-human CD3/CD28 magnetic dynabeads (ThermoFisher) at a beads to cells concentration of 3:1, along with a cytokine cocktail of IL-2 at 200 U/mL (UPenn hospital pharmacy), IL-7 and IL15 at 5 ng/mL (ThermoFisher).

Electroporation of Cas9-gRNA-Donor DNA (dsDNA or ssDNA)

Two days after T-cell activation, cells were electroporated to enable site-specific knock-in using Cas9 protein. All electroporation experiments were performed on the 4D-Nucleofector™ System X Unit (Lonza, Basel, Switzerland) using the EH-115 program. Cas9 protein and gRNA (Table 3) were pre-complexed at a Cas9:sgRNA ratio of 2:1 and incubated for 10 min at room temperature (RT). T cells (1.0×10⁶) were re-suspended in 17 μL P3 buffer including supplement 1 (Lonza). Subsequently, 3 μL of Cas9-sgRNA complex was added together with 1 ul of dsDNA or ssDNA template donor (3 μg or 5 ug). The Cas9-sgRNA-dsDNA/ssDNA mix were added to the cell mixture and 24 μL was added to the transfection strip and electroporated. After electroporation, 80 μL of above media was added to the electroporation well. The cells were rested for 15 min at 37° C. and 5% CO₂ before being transferred into a 24-well, tissue culture plate with 1000 μL of recovery media. T cells were fed every 2 days and fresh IL-7 and IL-15 cytokines were added.

TABLE 3
sgRNAs for targeting IL-2 locus

| sgRNA name | Sequence |
|---|---|
| gRNA 1 (antisense) | GAGTTGCATCCTGTACATTG |
| (SEQ ID NO: 173) | |
| gRNA 2 (sense) | CAACTCCTGCCACAATGTAC |
| (SEQ ID NO: 174) | |

Genomic DNA Extraction and PCR

Genomic DNA was extracted, and PCR was performed with primers (Table 1) to confirm that CAR19 gene integrated into the endogenous gene locus of interleukin IL2.

In Vivo Experiments

The in vivo experiments used the Nalm6 leukemic model previously described to demonstrate the functional nature of the human orthogonal IL2 system (Zhang, et al., Sci Transl Med, 2021. 13(625): p. eabg6986). NSG mice were engrafted with 1e6 CBG-labeled CD19+ Nalm6 leukemic cells on day 0. Mice received 1e6 CAR T cells (transduced, edited, and transduced with edited) were injected on day 5 following BLI on day 4. Tumor burden was assessed via bioluminescent imaging twice per week and CAR T cell expansion was examined weekly for 3 to 4 weeks. Mice received PBS or 20K or 40K IU of oIL2. In a second set of in vivo experiments, mice received PBS or 20K IU of oIL2 and CART cell expansion was examined weekly for 3 weeks.

The Results of the Experiments are Now Described

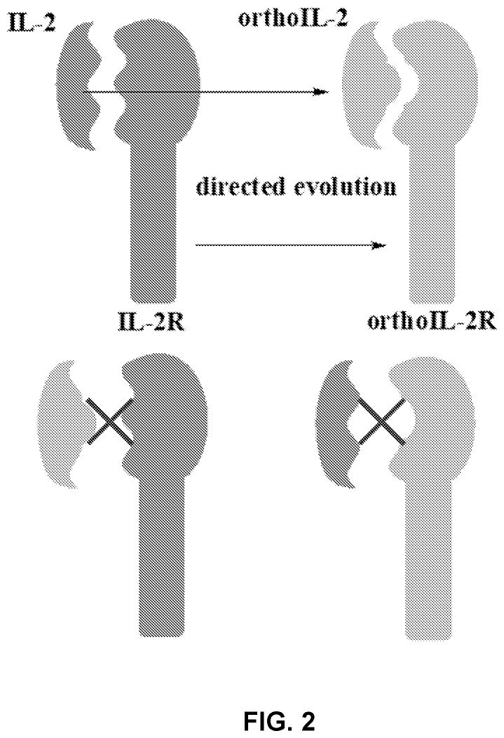

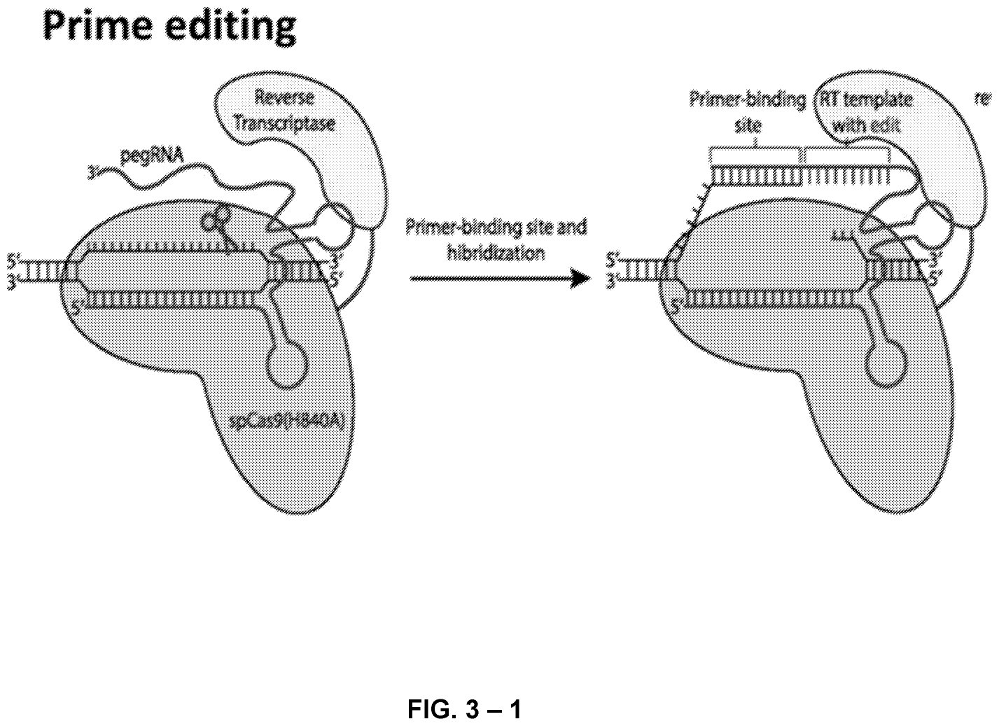

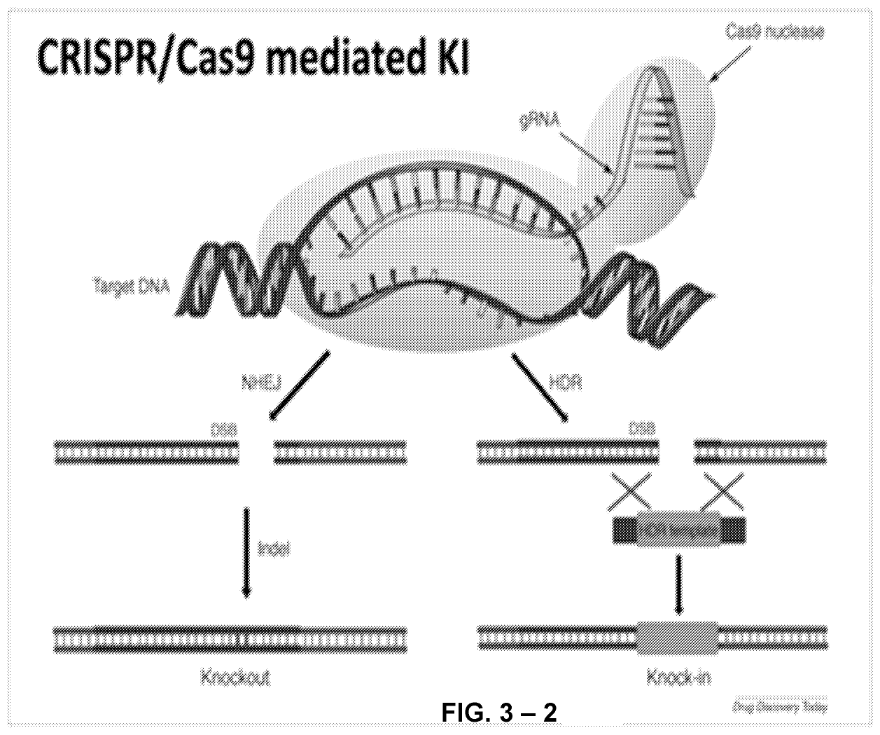

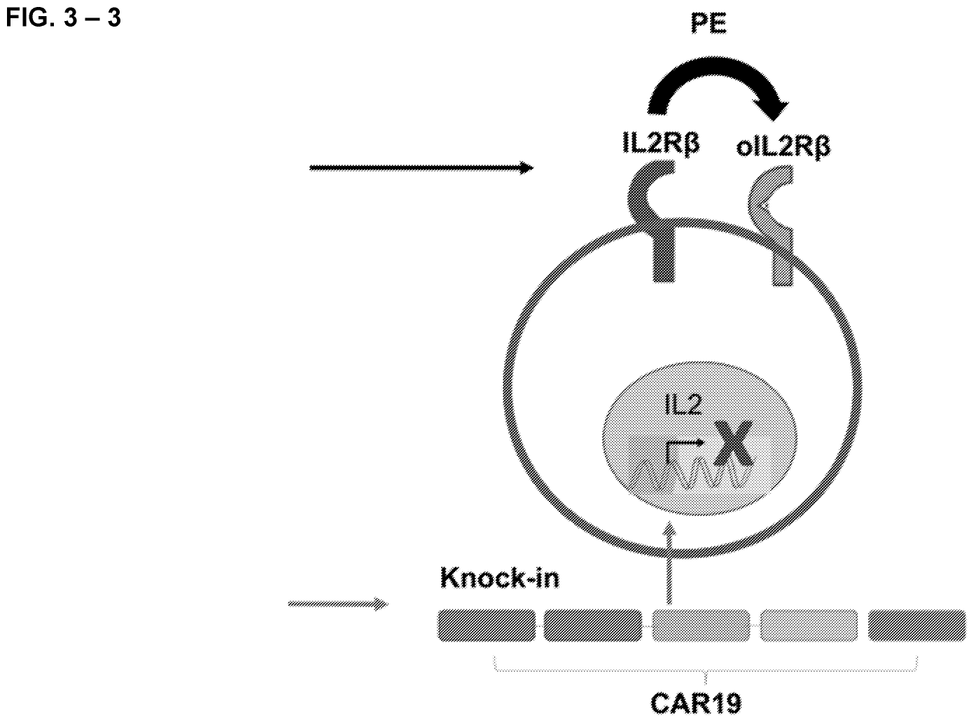

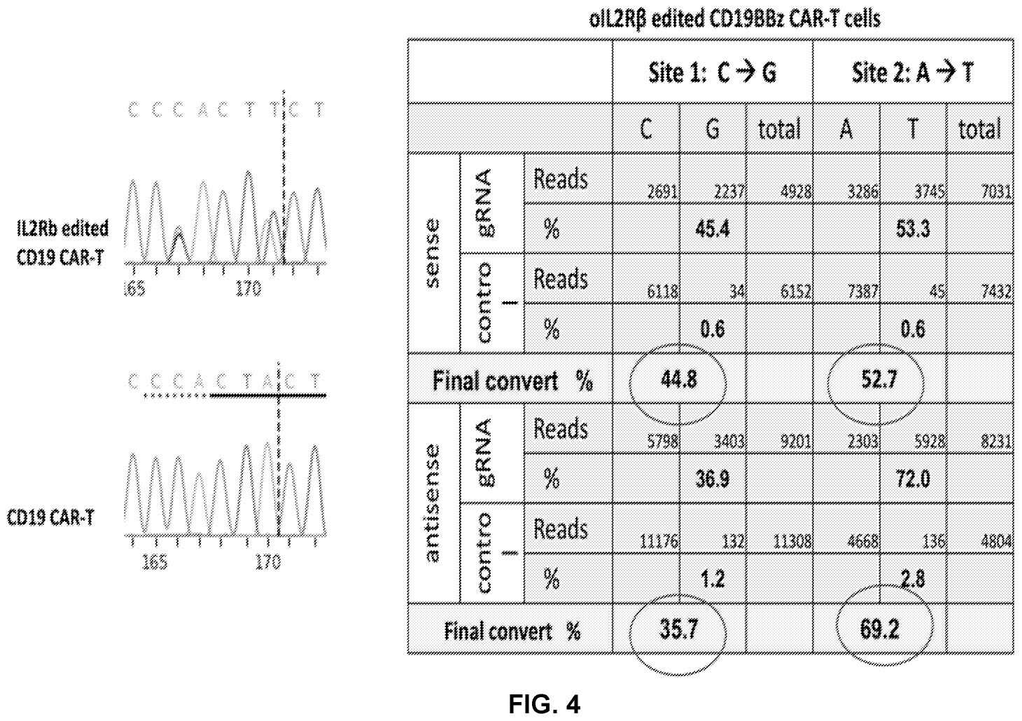

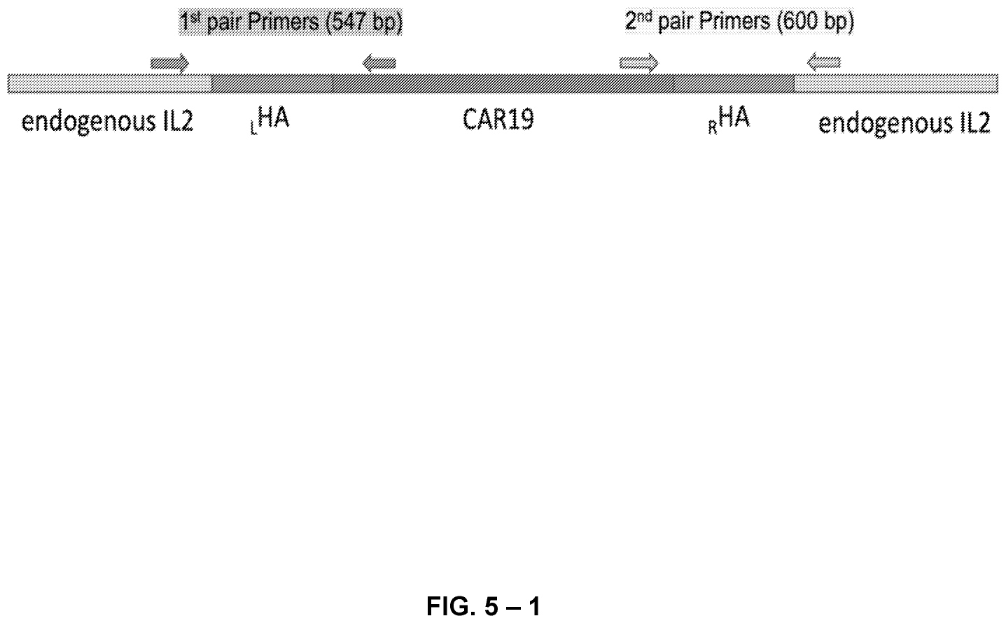

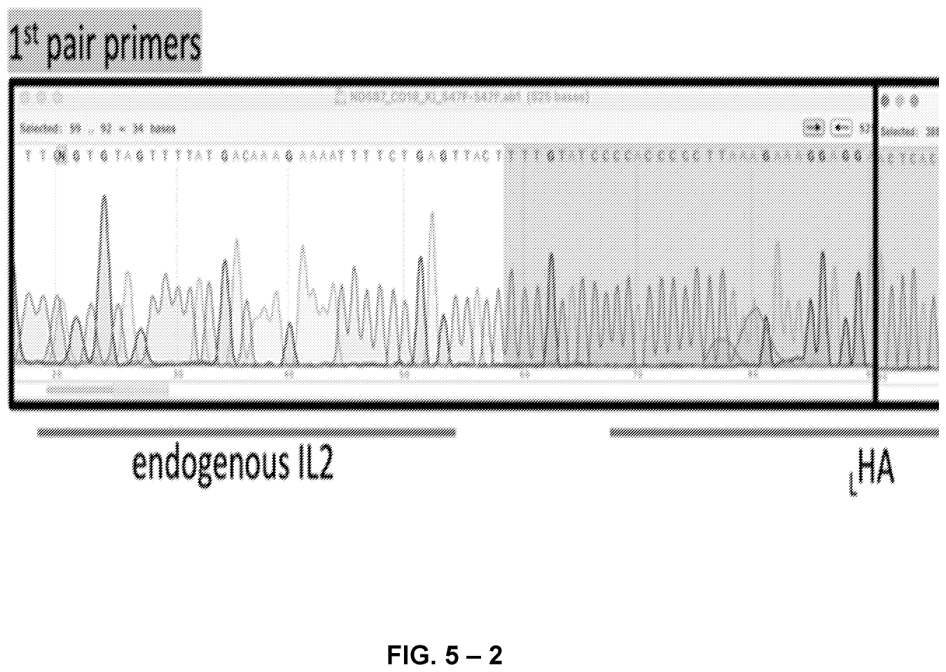

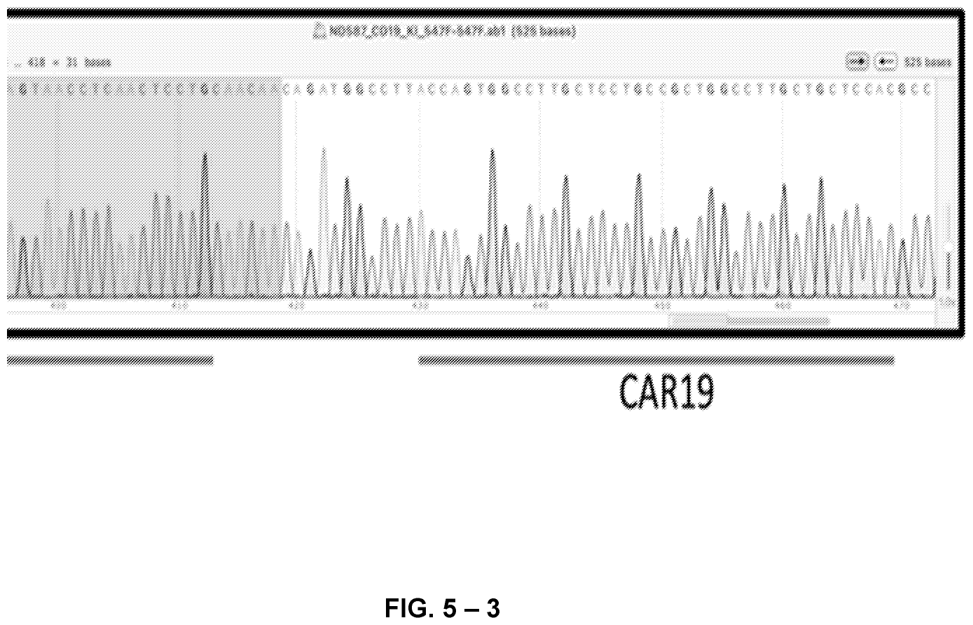

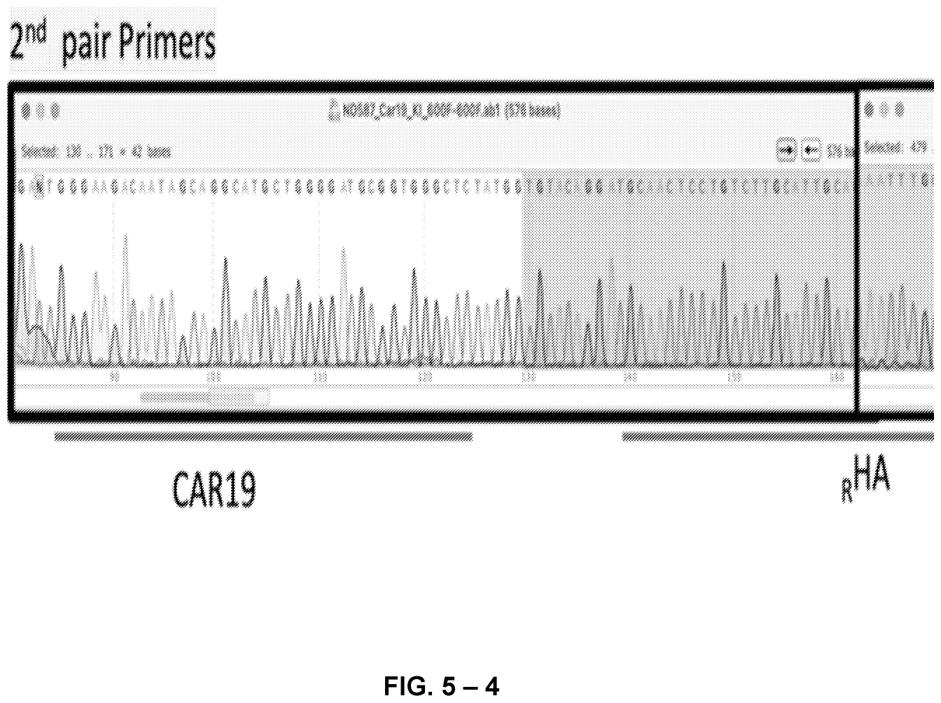

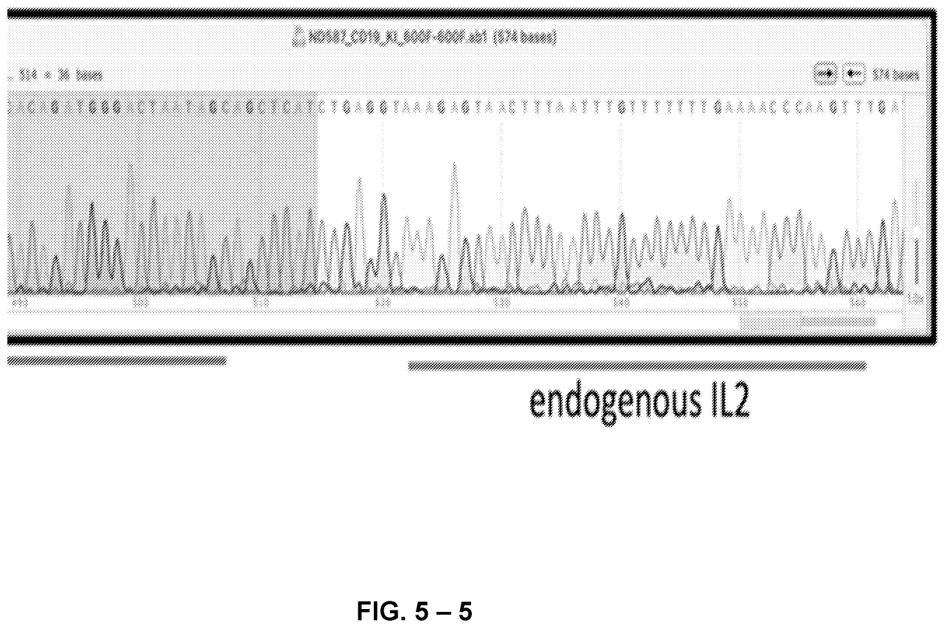

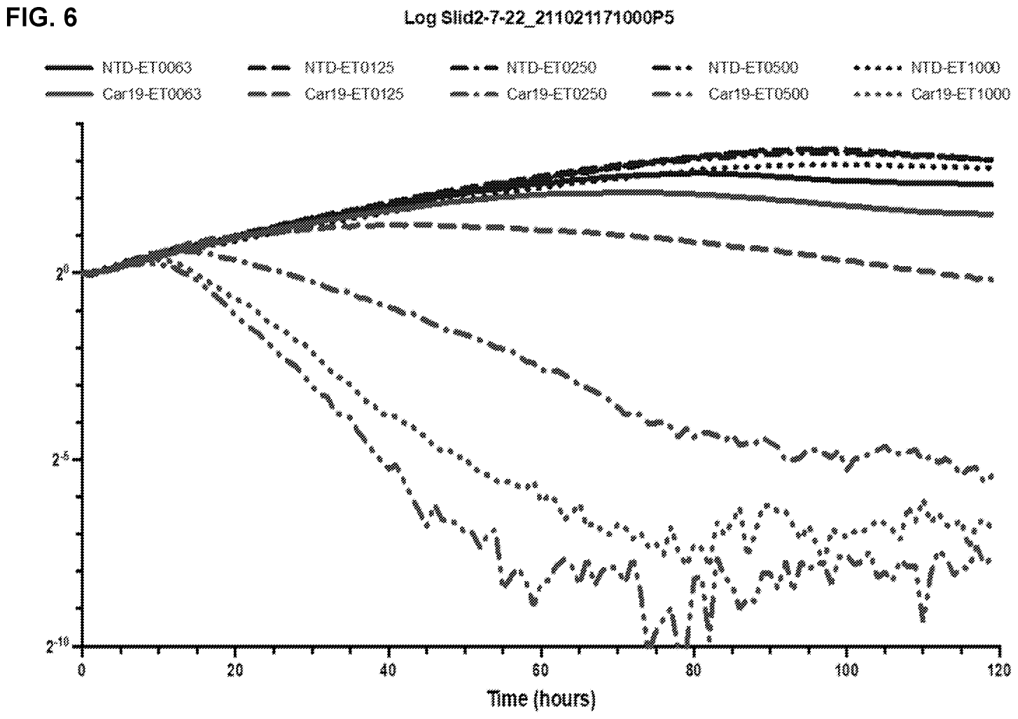

The invention disclosed here comprises IL-2/IL-15 responsive human immune effector cells, such as a T cell, that is engineered to express a CAR that is introduced into the IL-2 gene by CRISPR/Cas9-mediated homology directed repair (HDR), along with an orthoIL2Rb receptor (FIG. 2) introduced by mutation of the endogenous IL2Rb genes through prime editing. The HDR simultaneously disrupts the IL-2 gene while introducing the CAR to this genetic site. The engineered orthoIL2Rb+IL2−/− CAR T cell is shown schematically in FIG. 3. This orthoIL2Rb+IL2−/− CAR T cell was successfully generated. The presence of the oIL2Rb gene mutations (i.e., C397G and A401T) introduced via prime editing were confirmed by sequencing (FIG. 4). The functionality of these mutations (which result in H133D Y134F orthogonal IL2Rβ) was confirmed through response to orthoIL-2 in the IL-2 dependent SeAx T cell line (data not shown). Greater than 90% IL-2 gene disruption using CRISPR/Cas-9 mediated double strand breaks and introduction of the functional CAR19 was confirmed by sequencing (FIG. 5). Expression of CAR19 was further validated by demonstration of the functional activity of the CAR T cells generated using this viral vector-free gene editing process (FIG. 6).

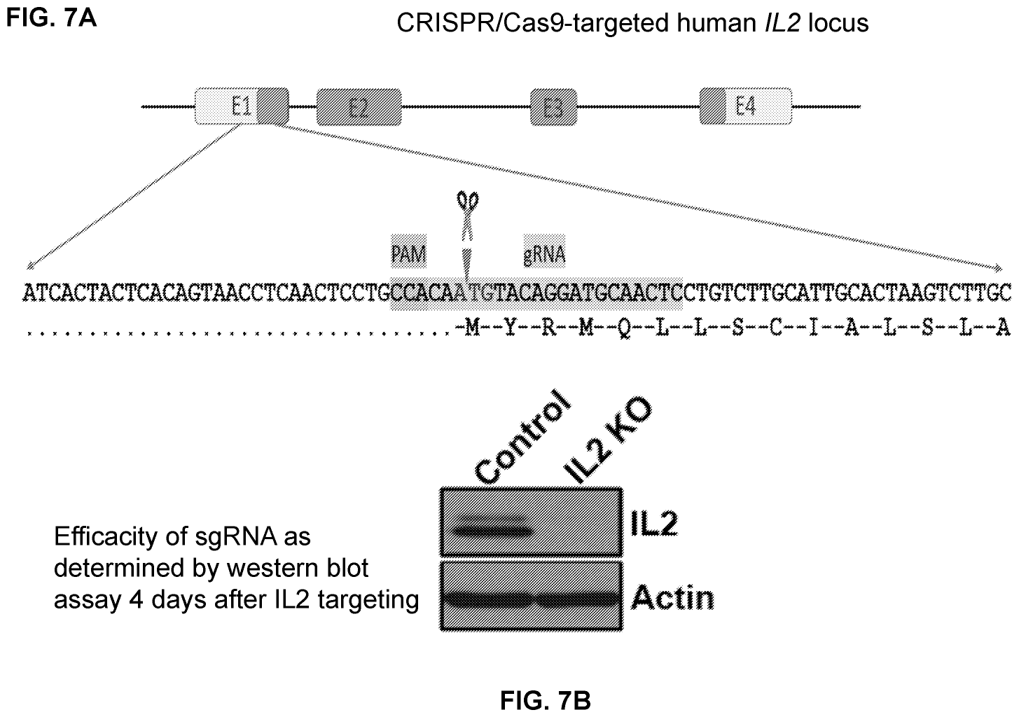

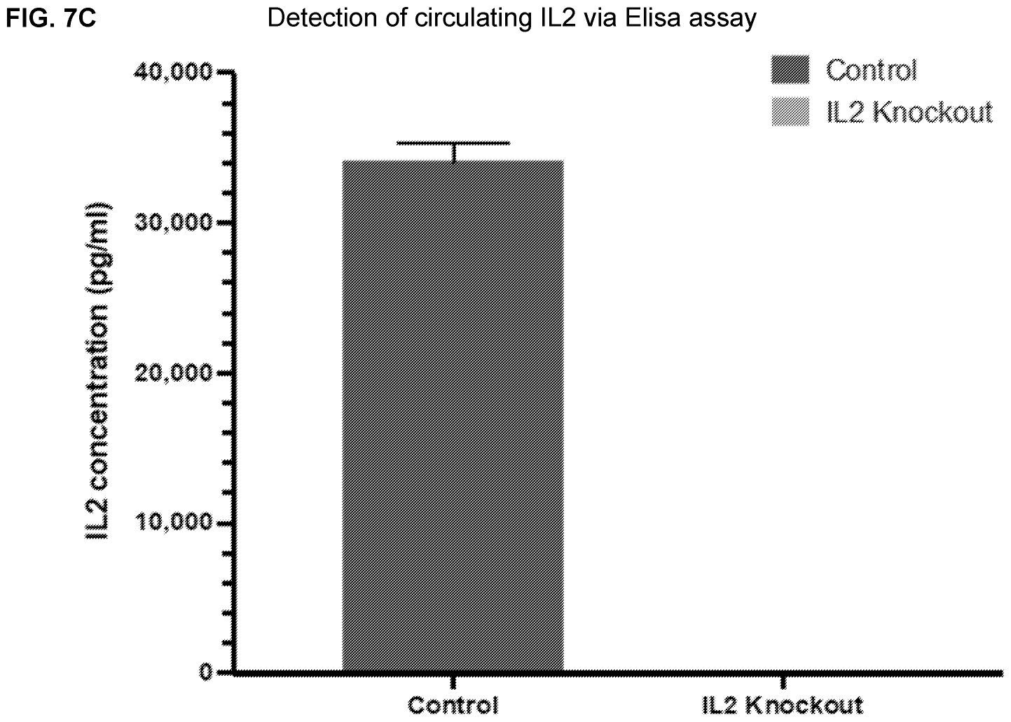

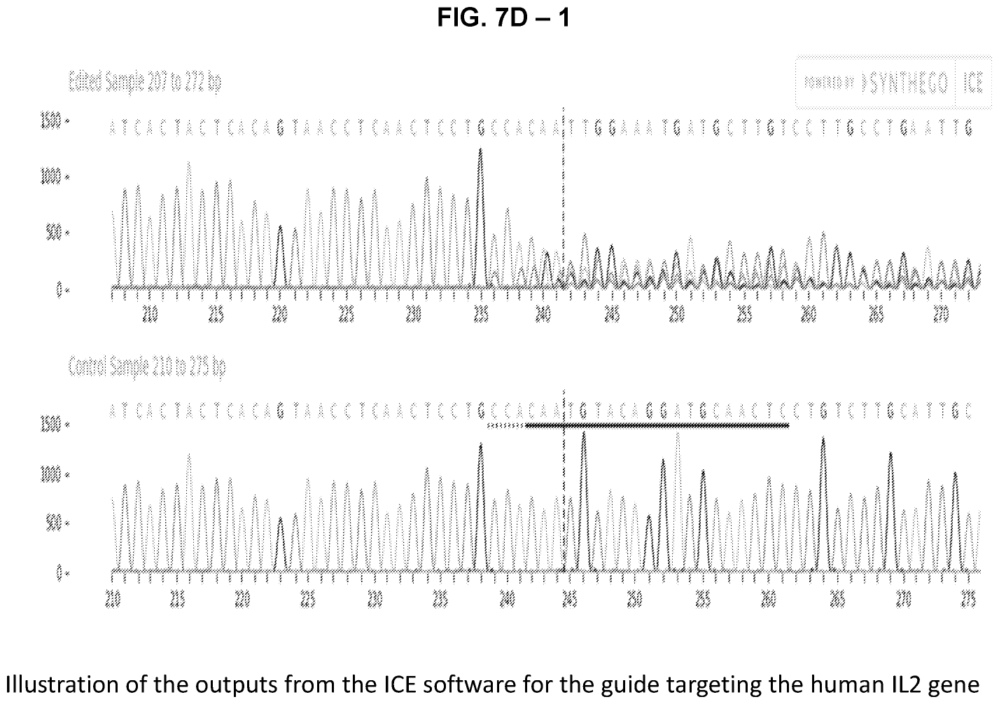

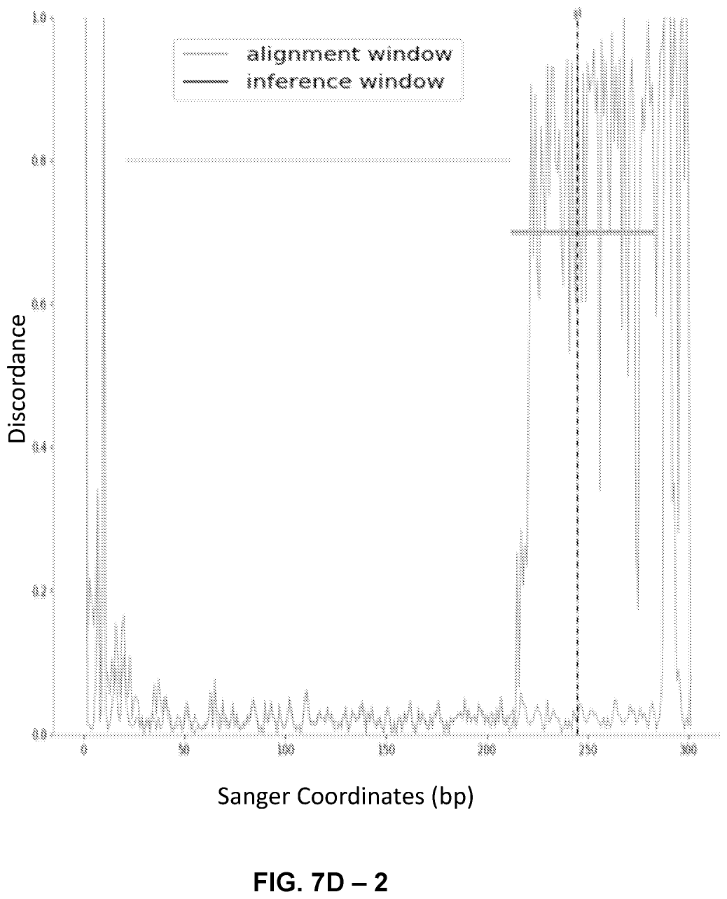

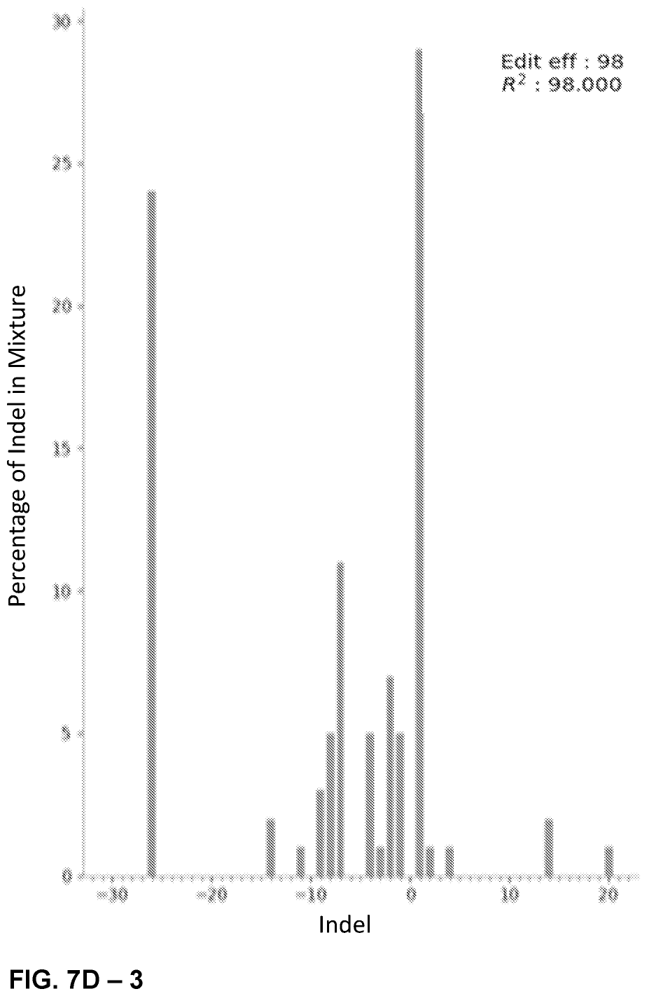

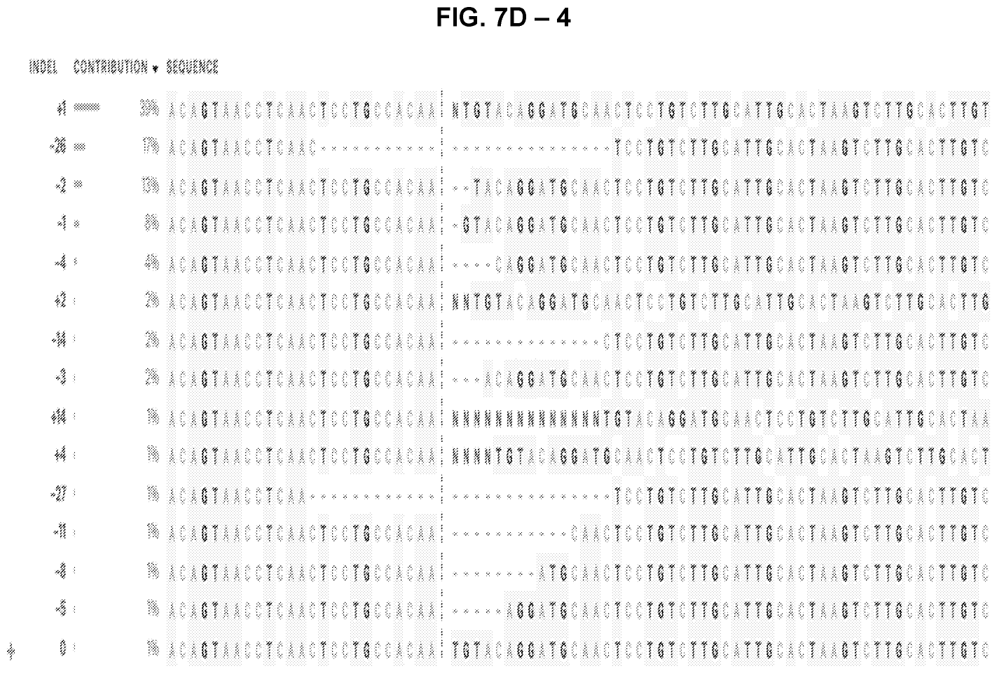

A schematic diagram of sgRNA targeting at the hIL2 Exon 1 locus is shown in FIG. 7A. SEQ ID NO: 112 and SEQ ID NO: 113 are shown. The sgRNA targeting site (SEQ ID NO: 114) on the antisense strand is highlighted, the protospacer adjacent motif (PAM) sequence (CCA) is labeled, and the expected cleavage site within the translation initiation codon (ATG) is indicated by the vertical arrowhead. Detection of sgRNA:Cas9-mediated cleavage of hIL2 from cells by western blot analysis is shown in FIG. 7B. Circulating IL2 from supernatant was detected via Elisa assay (FIG. 7C). ICE (Inference of CRISPR Edits) software was utilized for the guide targeting the human IL2 gene (FIG. 7D).

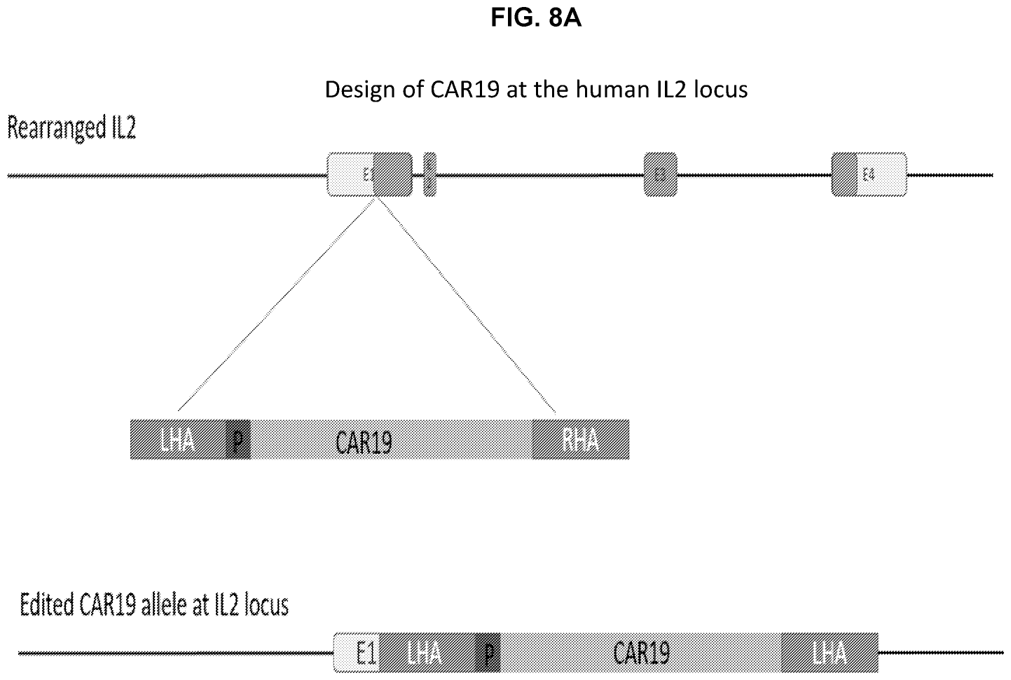

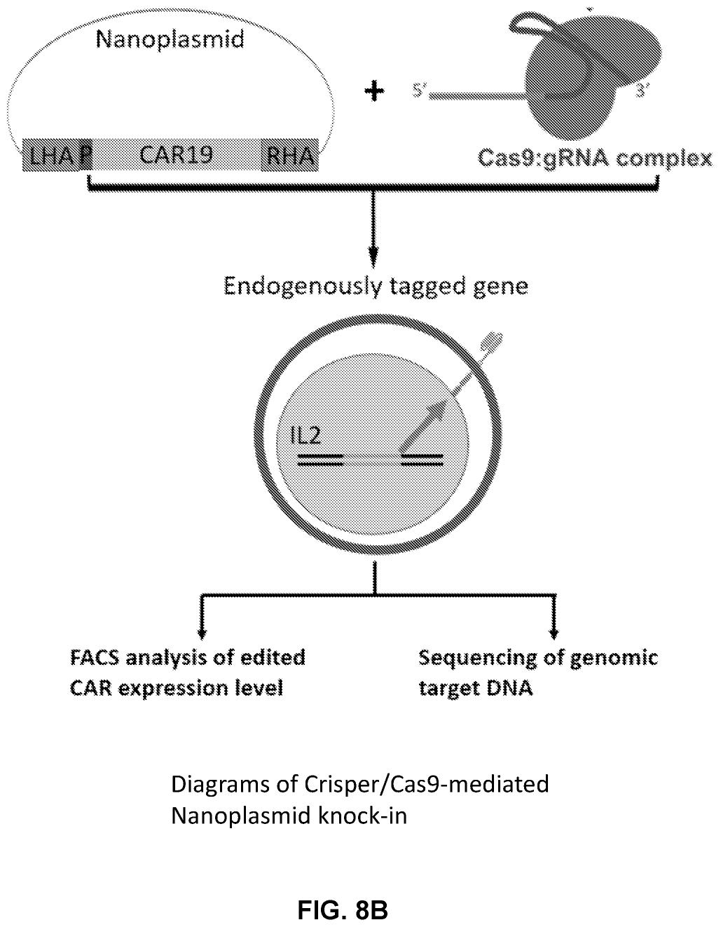

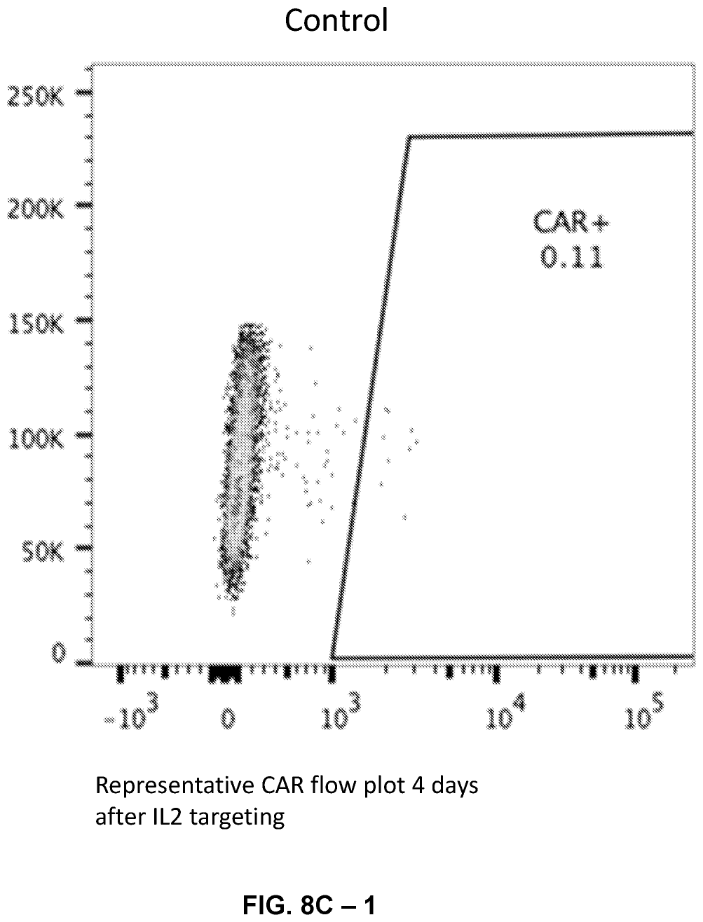

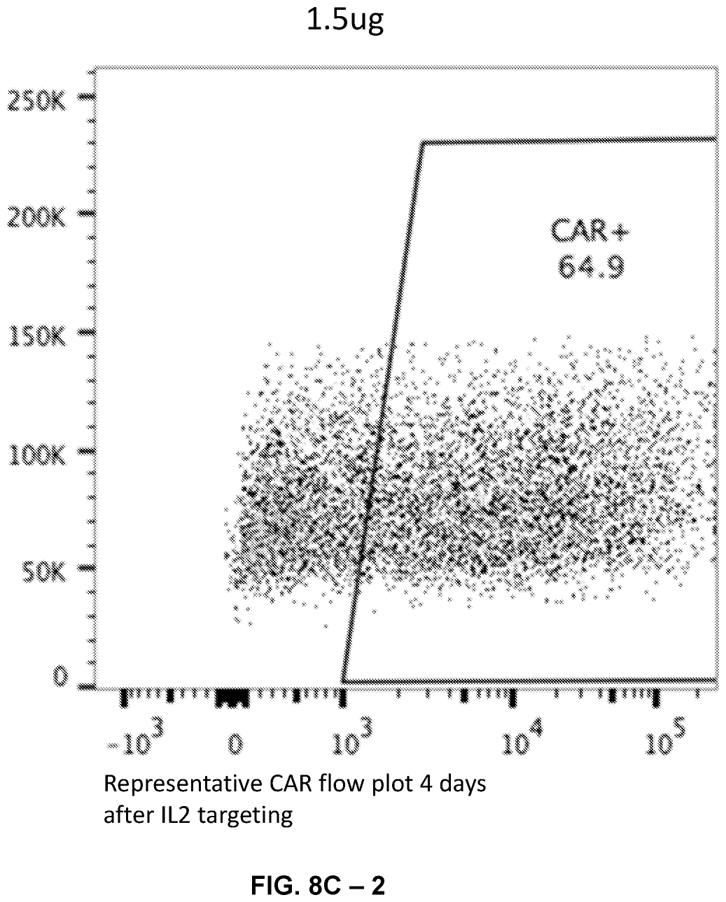

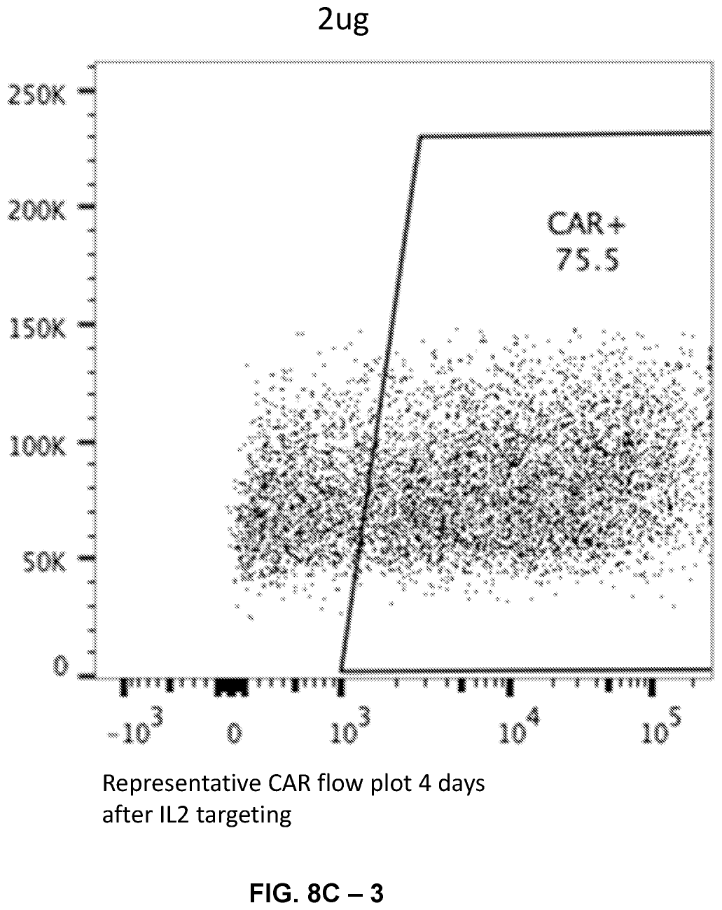

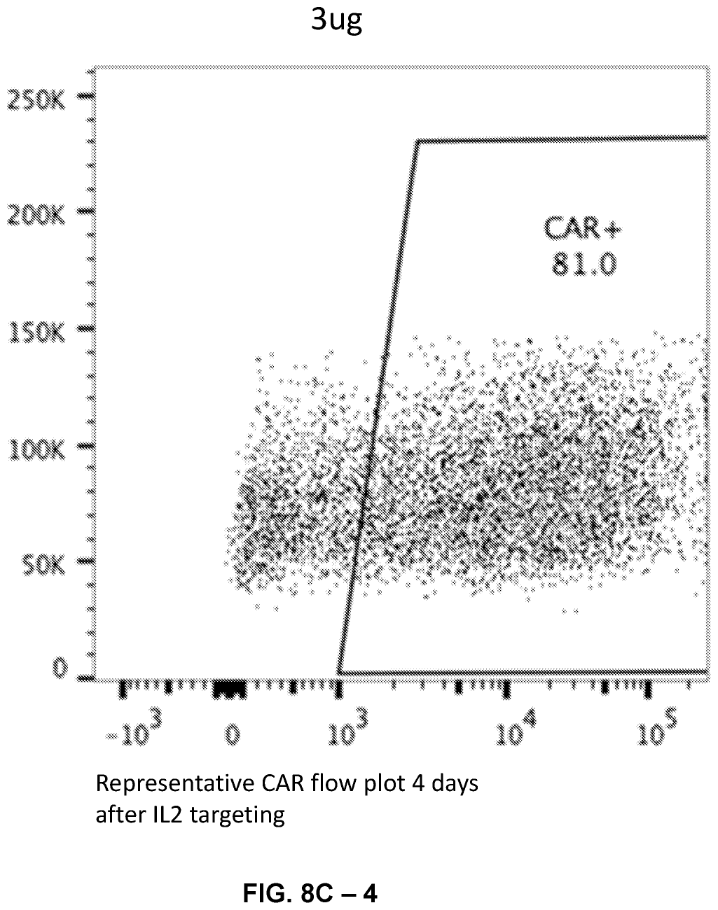

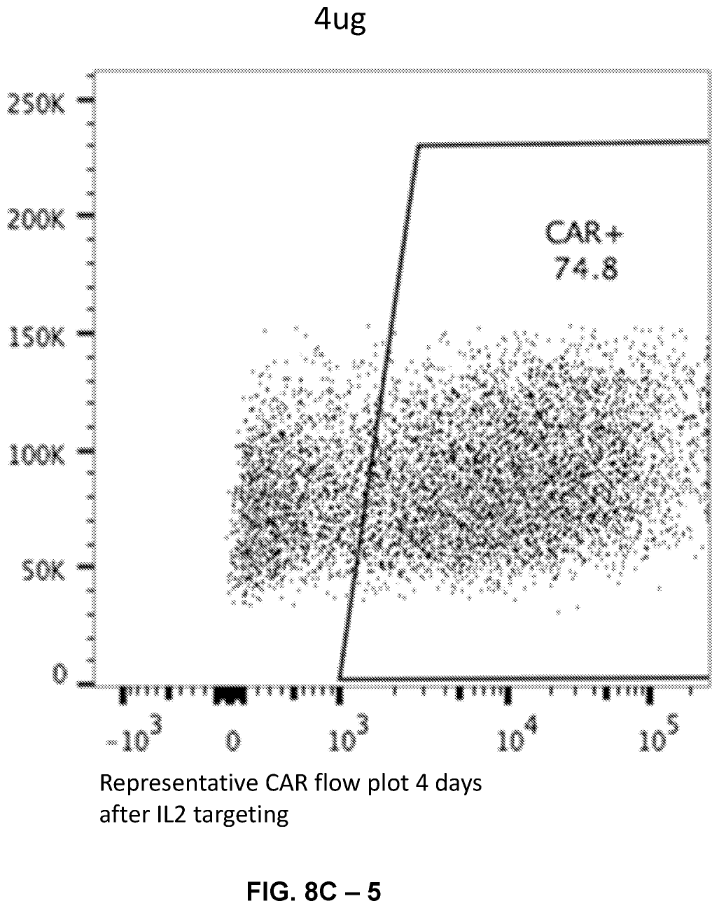

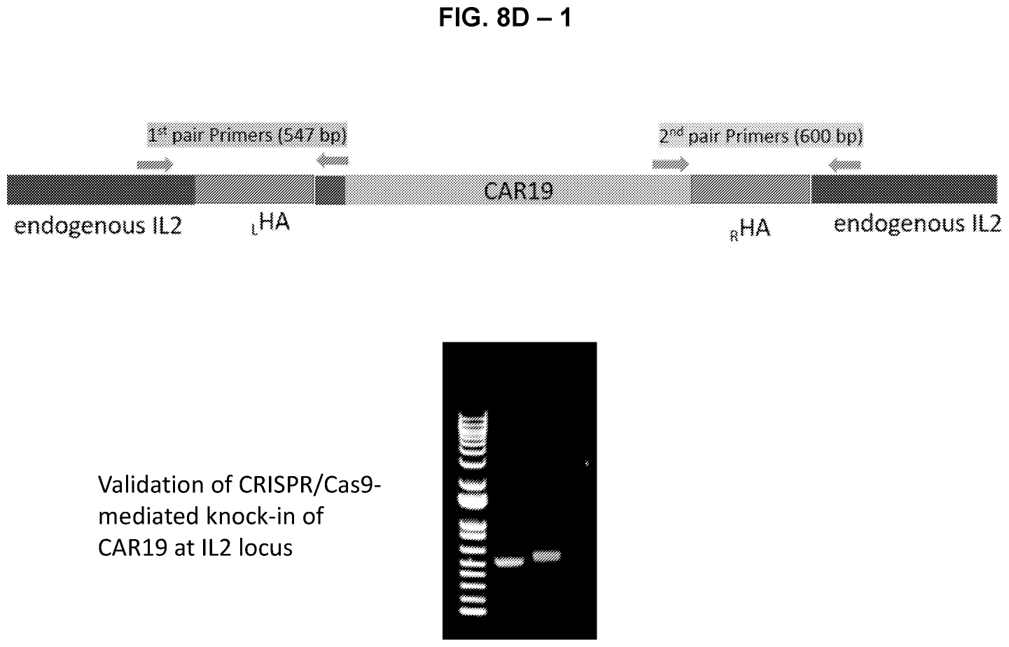

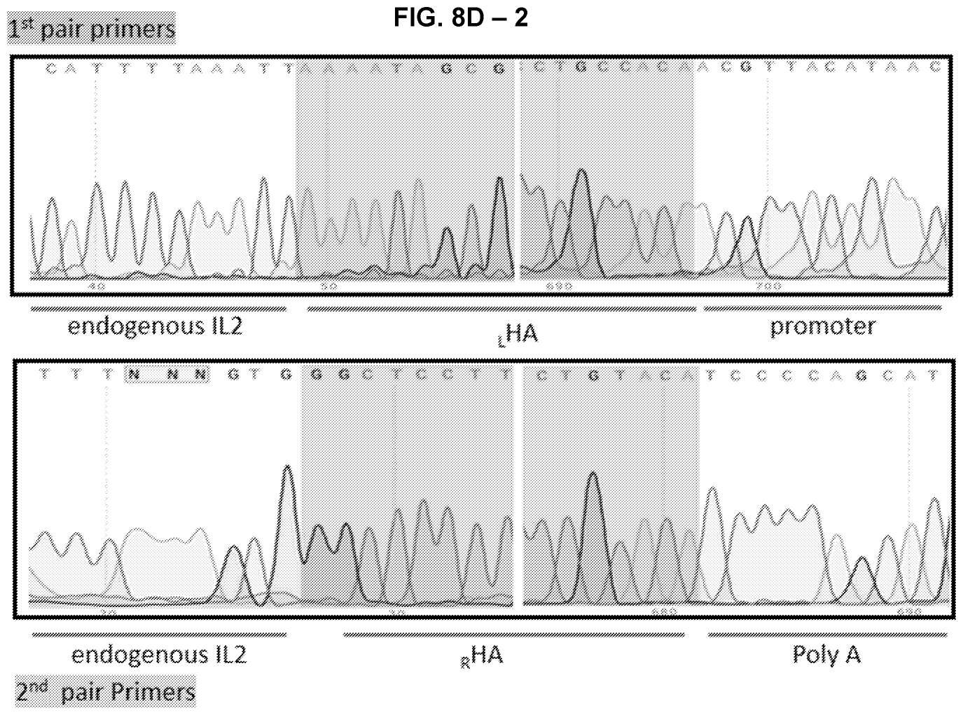

A schematic representation of the design to edit CAR into the human IL2 locus using CRISPR/Cas9-targeted CAR19 gene integration with promoter-containing donor plasmid DNAs is shown in FIG. 8A. A schematic representation of Cas9:single-guide RNA ribonucleoprotein (Cas9 RNP) delivery to primary human T cells for genome editing, followed by genetic and phenotypic characterization is shown in FIG. 8B. CAR FACS flow plots 4 days after IL2 targeting show increasing percentages of CAR19 with higher concentrations of donor DNA compared with control-treated cells (Cas9 without sgRNA) (FIG. 8C). Validation of CRISPR/Cas9-mediated knock-in of CAR19 at IL2 locus is shown in FIG. 8D. First panel shows a schematic indicating the position of two-pair primers flanking the knock-in sites and an agarose gel showing PCR amplification of knock-in region using the two-pairs primers. Second panel shows DNA sequencing analysis of the amplified DNA fragments, which revealed that the CAR donor DNA was correctly knocked-in at the IL2 gene locus.

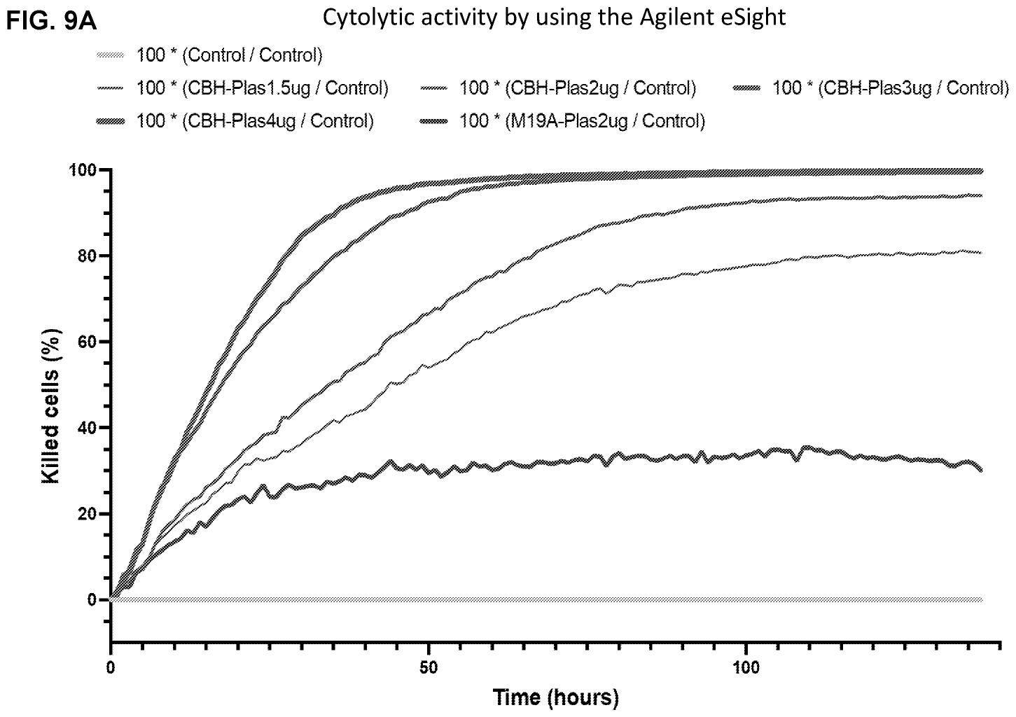

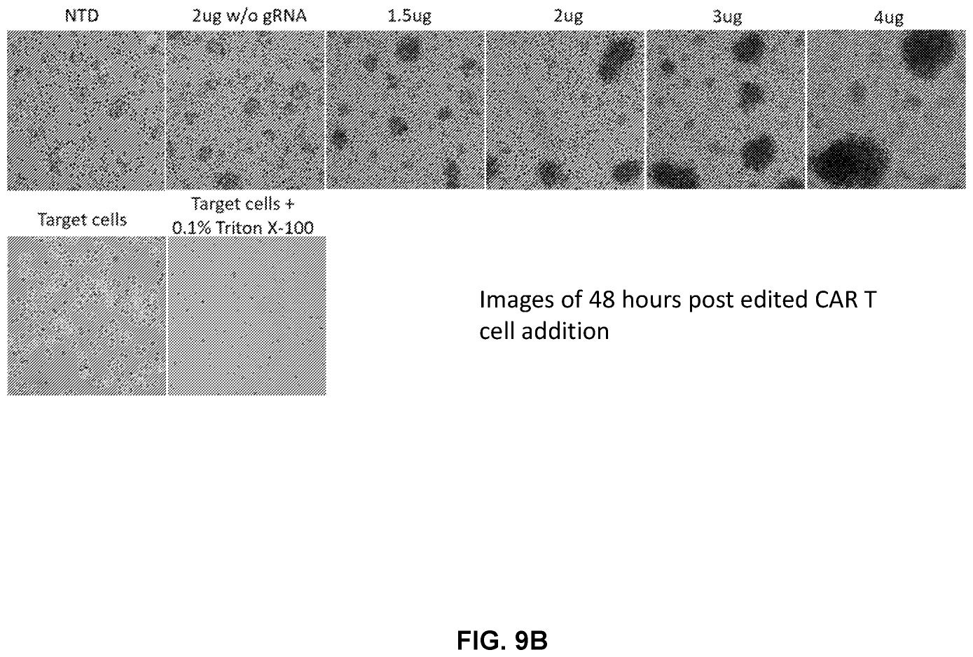

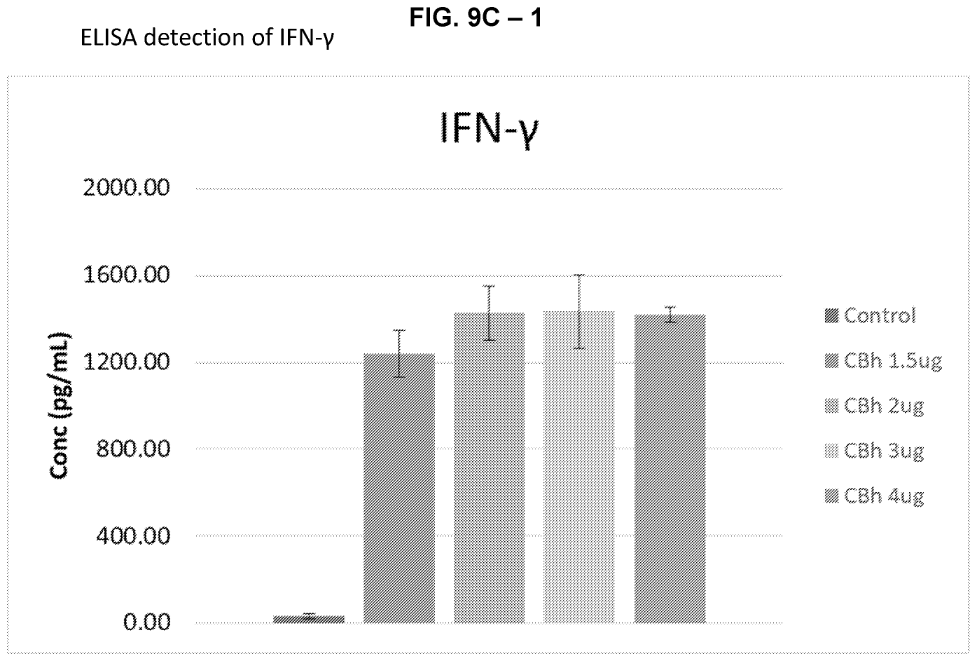

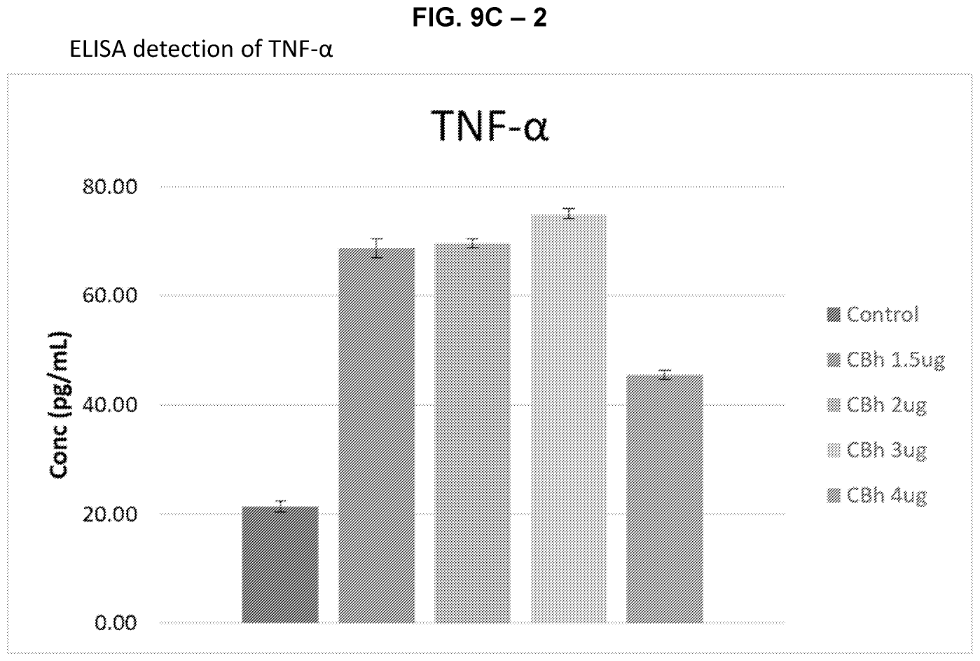

Next, cytotoxicity of the CAR19-engineered T cells was assessed using an image-based Agilent eSight assay (FIG. 9A). The data indicate that the engineered cells recognize and kill antigen-expressing target cells. Killing of GFP-expressing K562-CD19 cells by edited CD19 CAR-T cells with indicated doses of donor DNA concentration at a specific E:T ratio (5:1). Untreated target cells and target cells treated with 0.1% Triton X-100 (100% lysis control) were used as control. FIG. 9B shows time-dependent fluorescent images for GFP+K562-CD19 cells treated with edited CAR-T, as well as the unedited T cells. FIG. 9C shows two bar graphs showing IFN-γ and TNF-α production, respectively, by CAR19 knock-in T cells stimulated with indicated concentrations of donor DNA.

The optimal promoter and enhancer elements required for stable CAR expression and function when inserted into the IL2 gene will be determined using prolonged in vitro culture to assess stability. Promoters to be tested will include the long and short forms of EF1a, PGK and the MND U3 promoter.

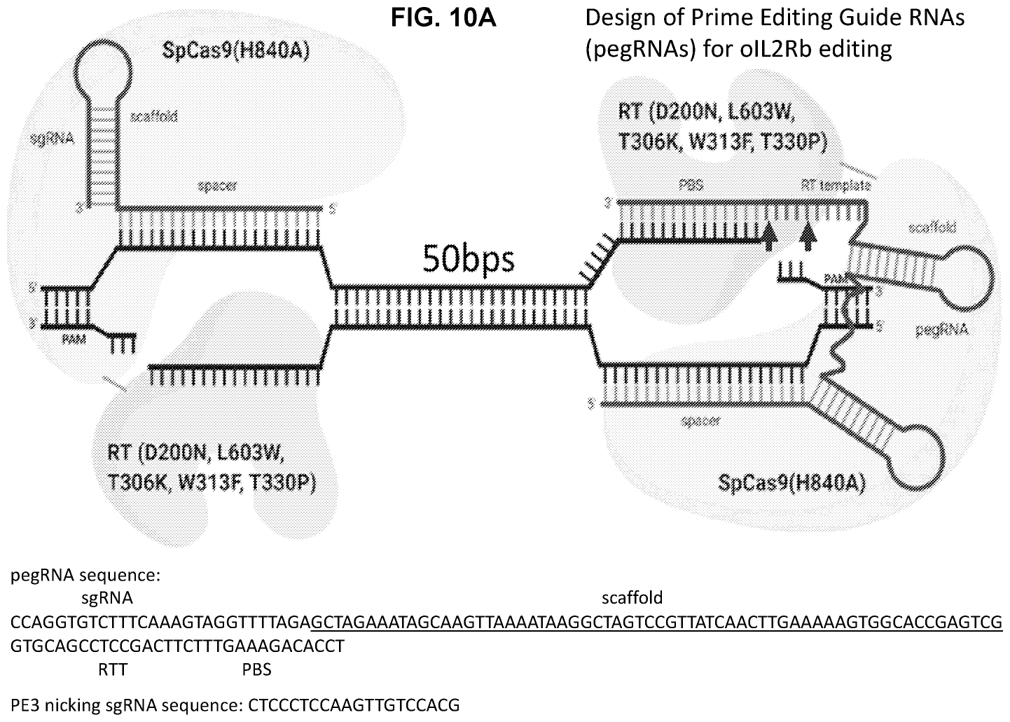

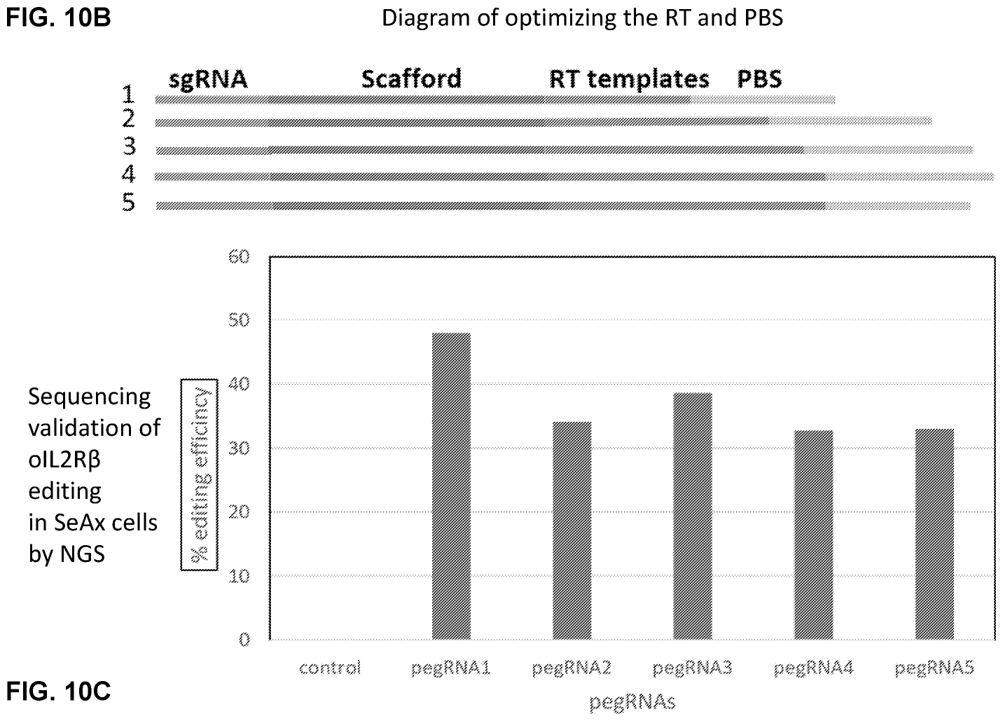

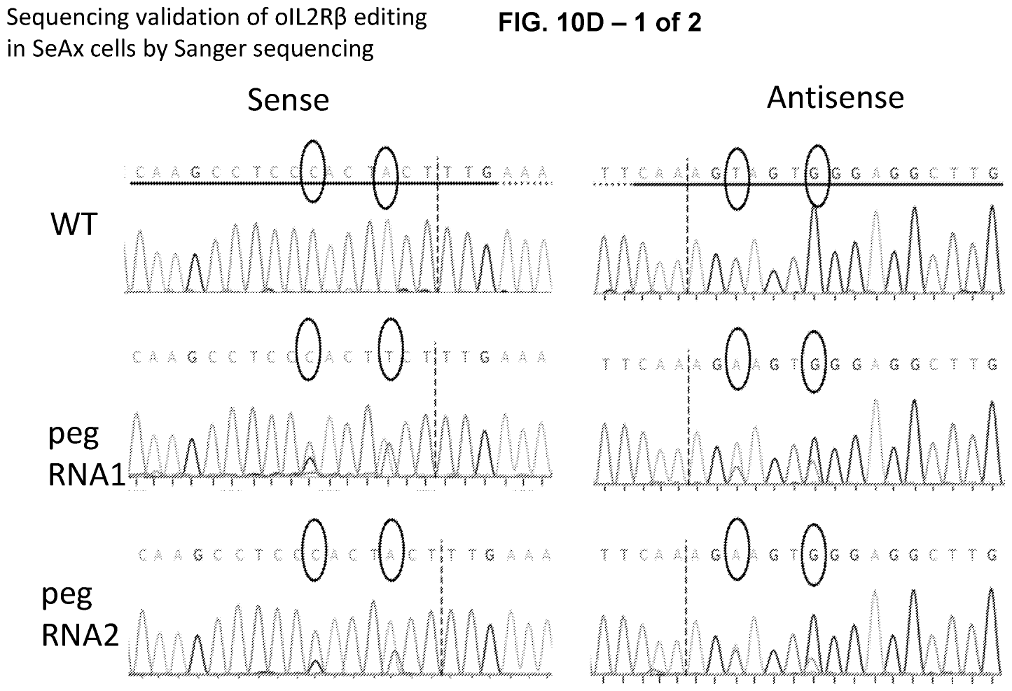

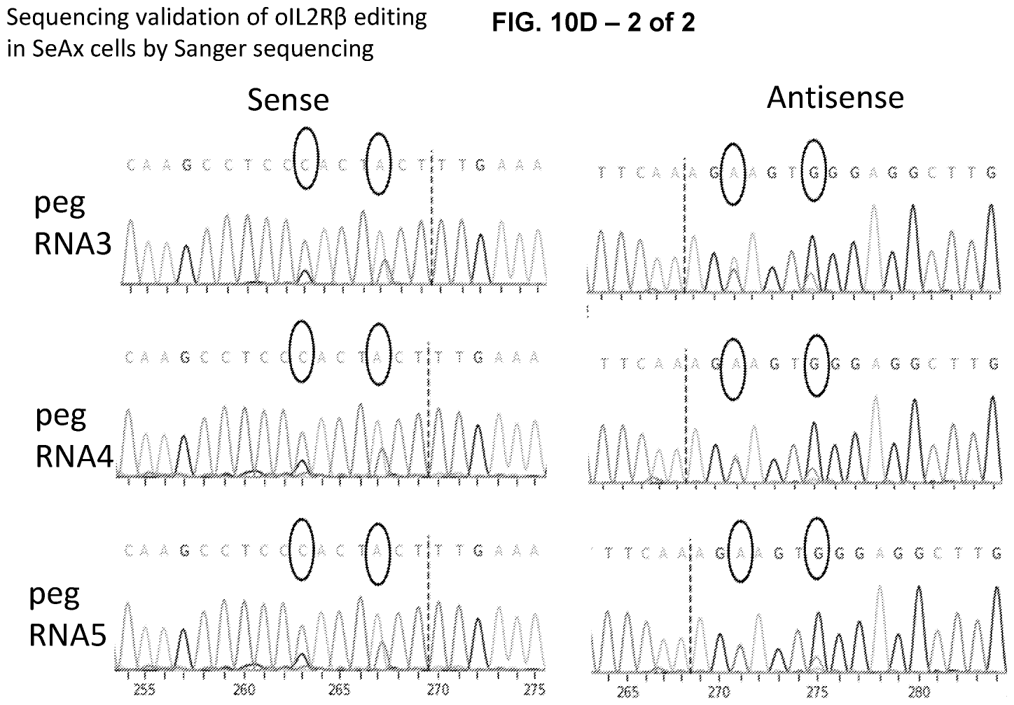

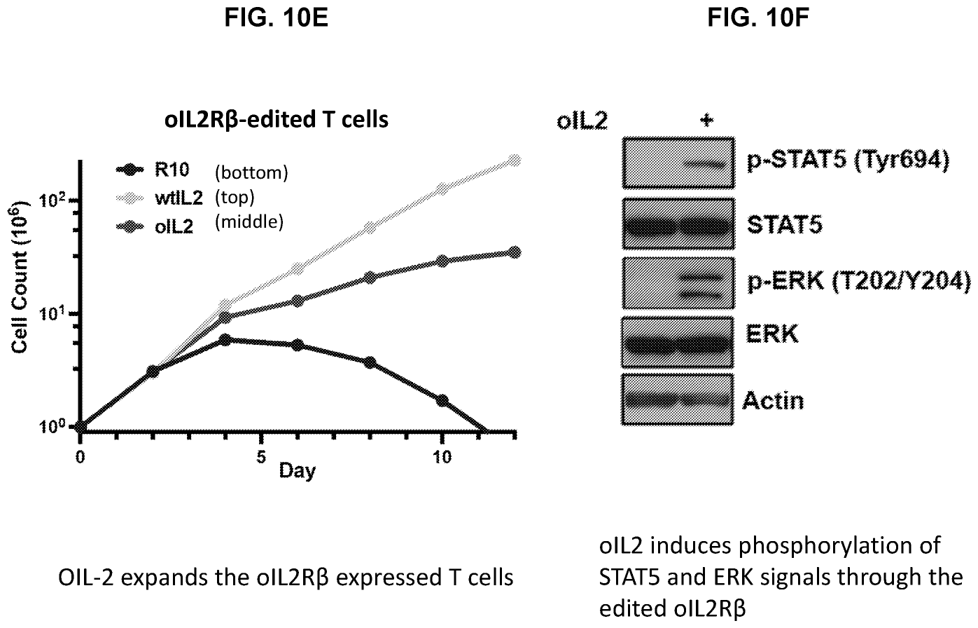

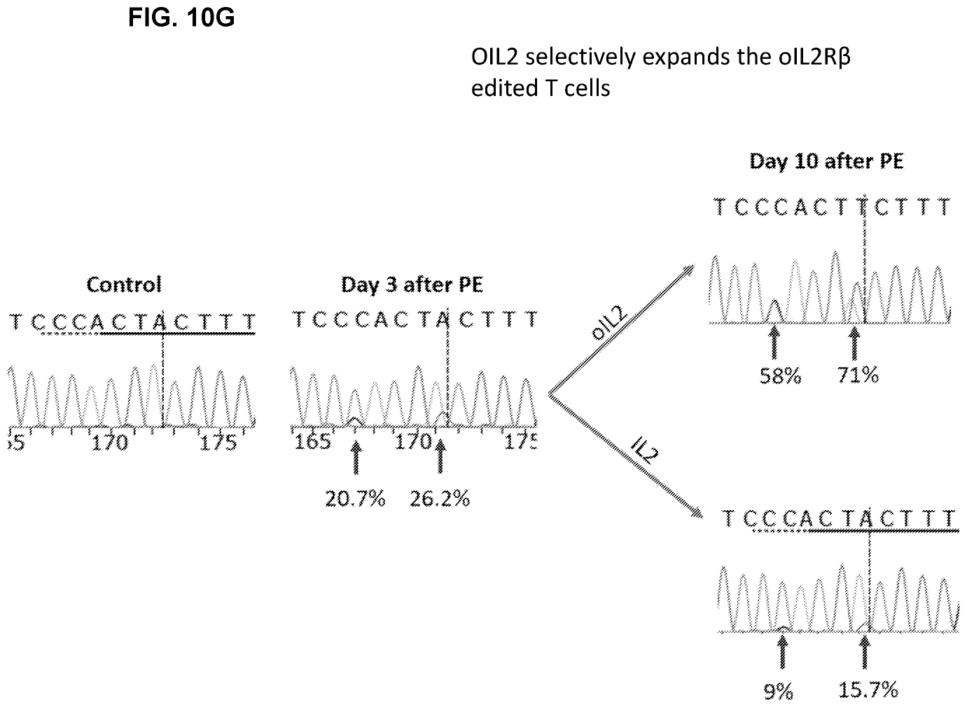

Next, prime editing of endogenous human IL2 receptor (IL2Rb) was used to generate the human orthogonal IL2Rb (oIL2Rb). A schematic diagram of prime editing using PE3 strategy which utilizes a pegRNA matching the target locus and a separate sgRNA that targets upstream of the edit site is shown in FIG. 10A. The full-length pegRNA sequence (SEQ ID NO: 26) is shown including sgRNA in blue (SEQ ID NO: 27), scaffold in underlined (SEQ ID NO: 28), PBS in yellow (SEQ ID NO: 30), and RT in green (SEQ ID NO: 29) with edit sites (red) and PE3 nicking sgRNA sequence (SEQ ID NO: 31). FIG. 10B is a diagram showing optimization strategy of five pegRNAs targeting wt-IL2Rb exon 1 with various RT and PBS. FIG. 10C is a chart showing prime editing efficiency by Next-Gen Sequencing (NGS). FIG. 10D shows sanger sequencing chromatograms of the PCR fragments from control and prime-edited cells with the five pegRNAs using IL2-dependant SeAx cells. FIG. 10E is a graph showing that oIL-2 expands oIL2Rβ edited human primary T cells (as well as oIL2Rβ edited SeAx cells (data not shown)). FIG. 10F shows that oIL2 induces the main signal pathways including phosphorylation of STAT5 and ERK through the edited oIL2Rβ with human primary T cells. FIG. 10G is sequencing data showing that oIL2 selectively expands the oIL2Rβ edited T cells. The oIL2Rb edited T cells increased in oIL2 culture and decreased in wt-IL2 culture.

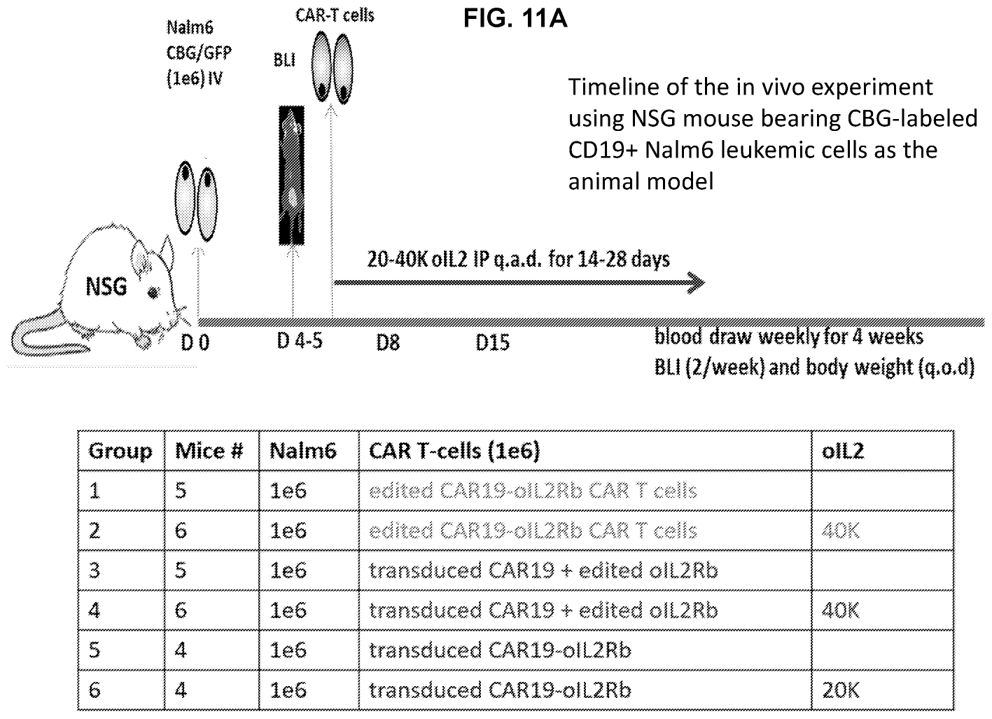

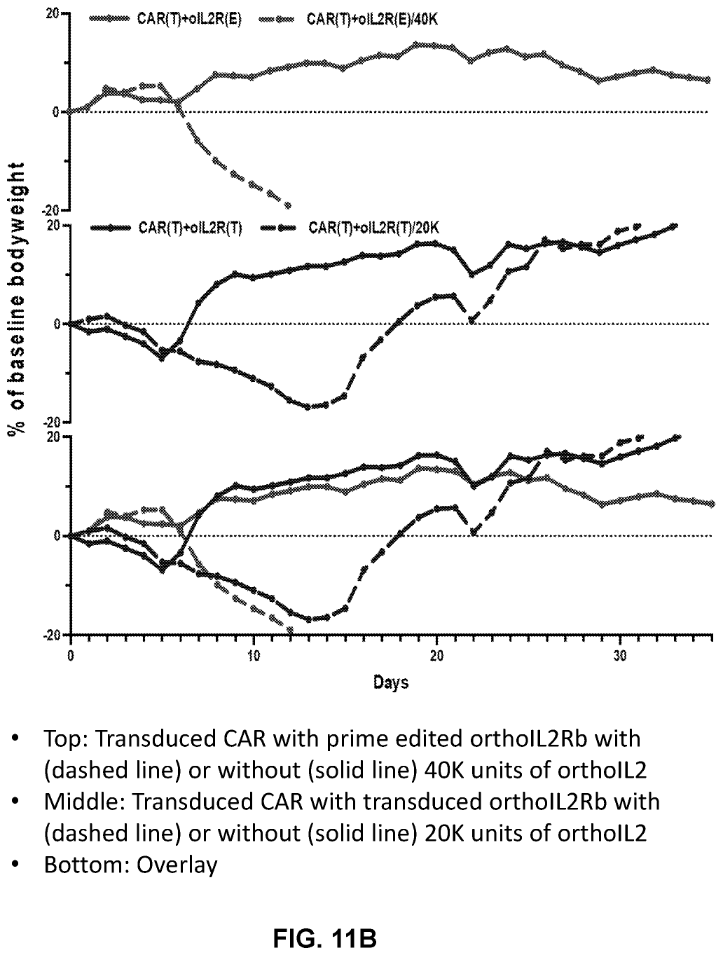

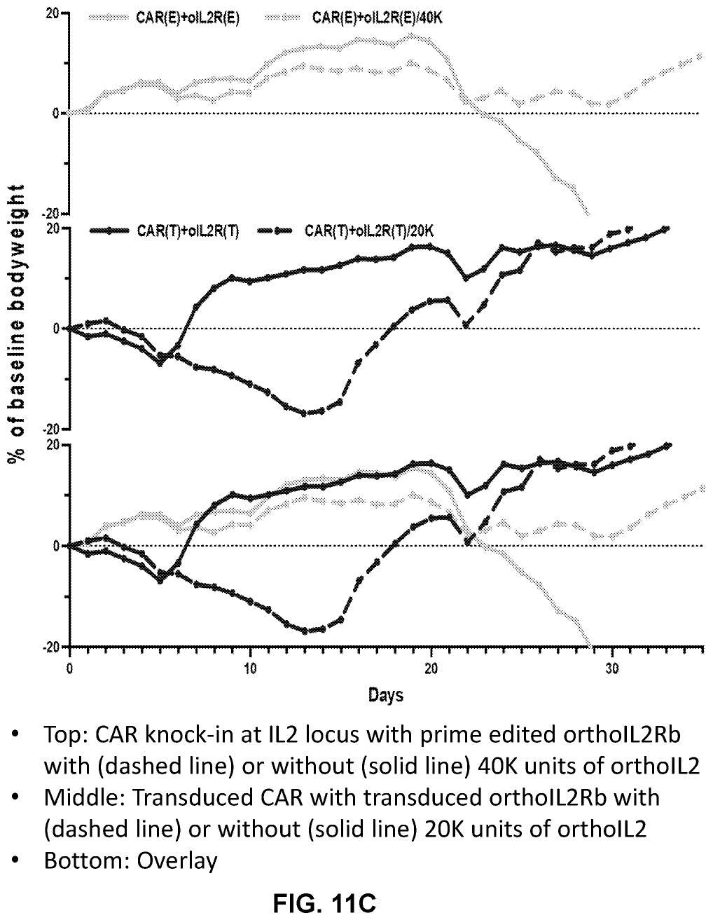

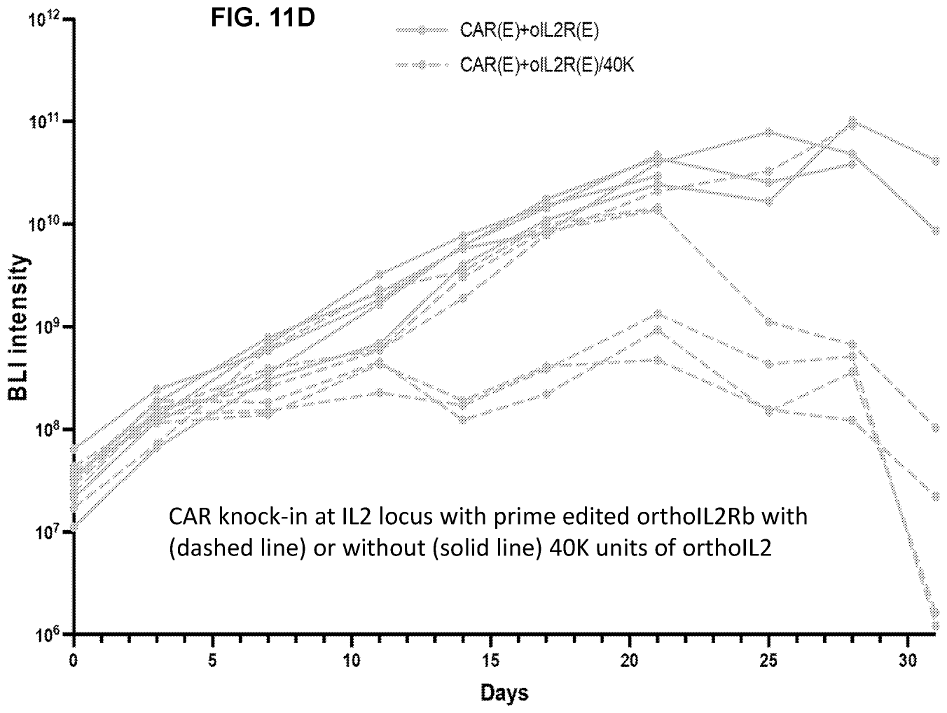

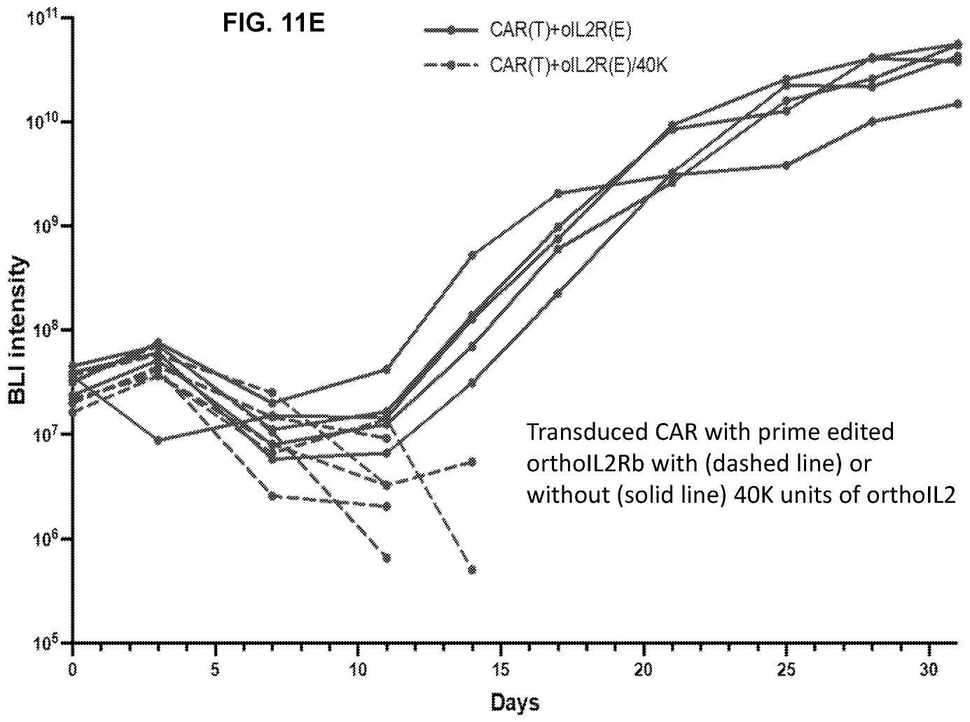

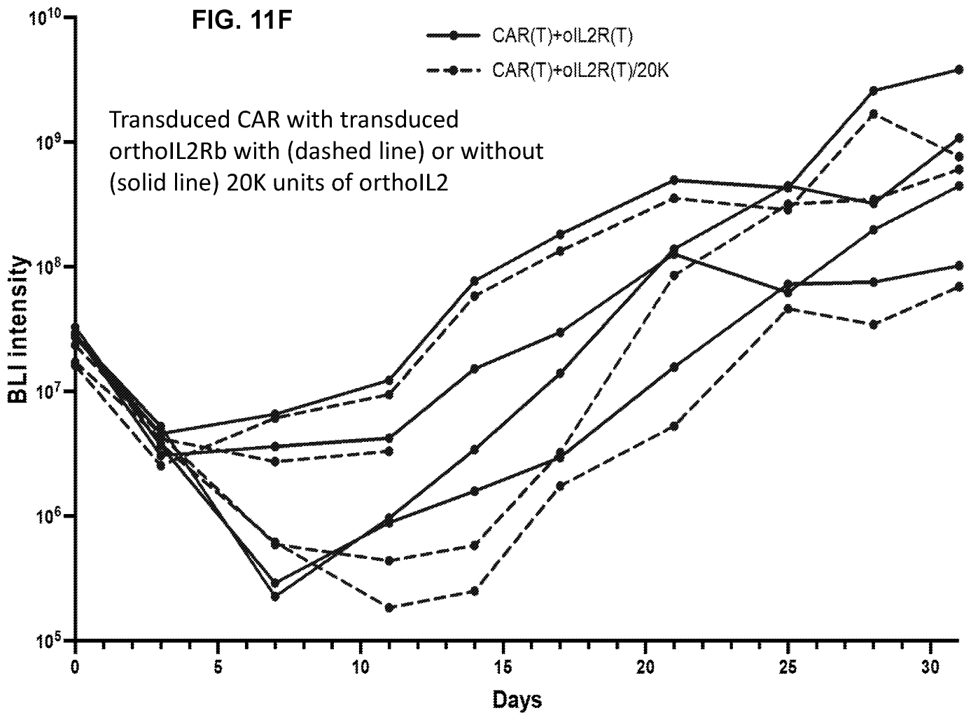

The engineered orthoIL2Rb+IL2−/− CAR T cell described herein (“edited” cells) were next compared in vivo with T cells transduced with a lentiviral vector to express both orthoIL2Rb and CAR19 (“transduced” cells), and with T cells edited to express oIL2Rb and transduced with a lentiviral vector to express CAR19 (“transduced with edited” cells) (FIG. 11A). Mouse body weight over time was normalized to the body weight on day 0 for each mouse receiving PBS or 20K or 40K IU of oIL2 (FIG. 11B and FIG. 11C). BLI intensity of Nalm6-LUC was determined for mice infused with the various T cells (edited, transduced, and transduced with edited) and receiving PBS or oIL2 (FIGS. 11D-11F). Together, the data indicate that the engineered orthoCAR19 T cells described herein show anti-leukemic activity.

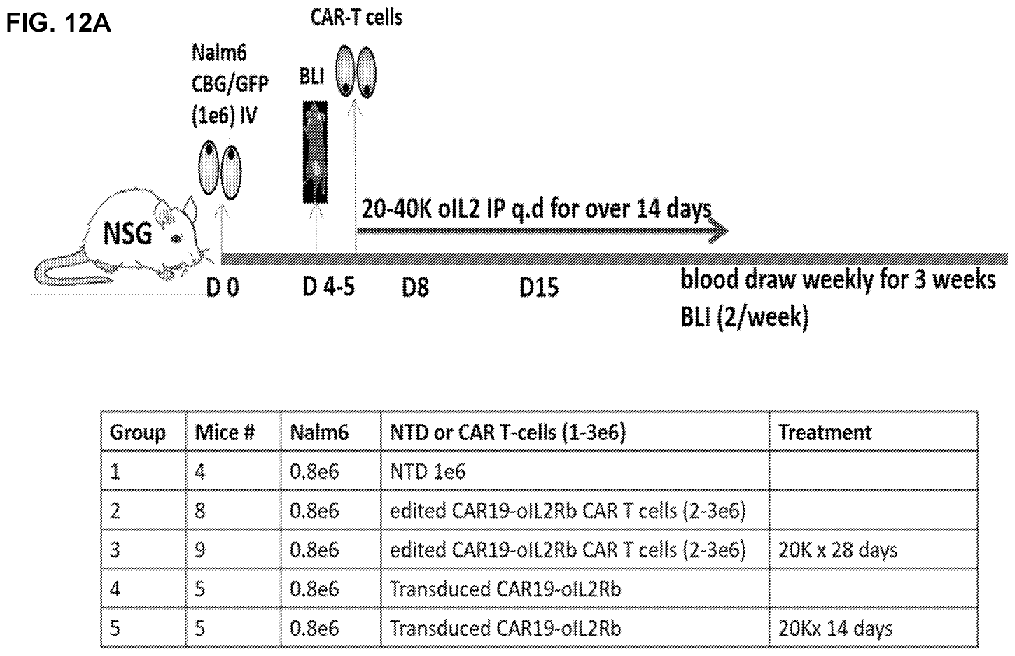

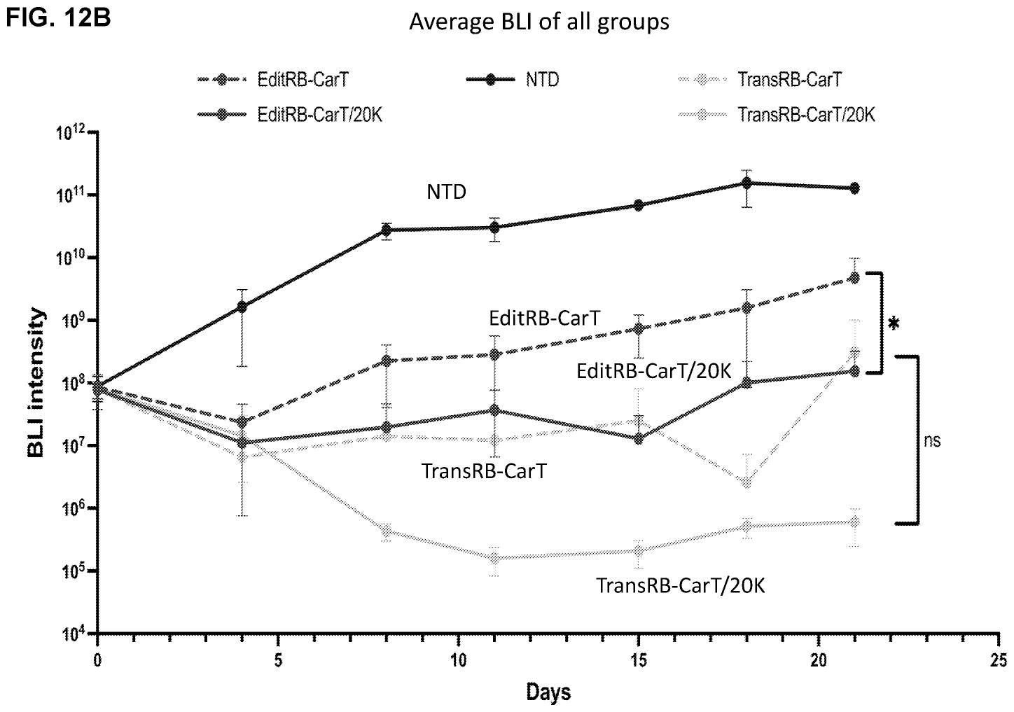

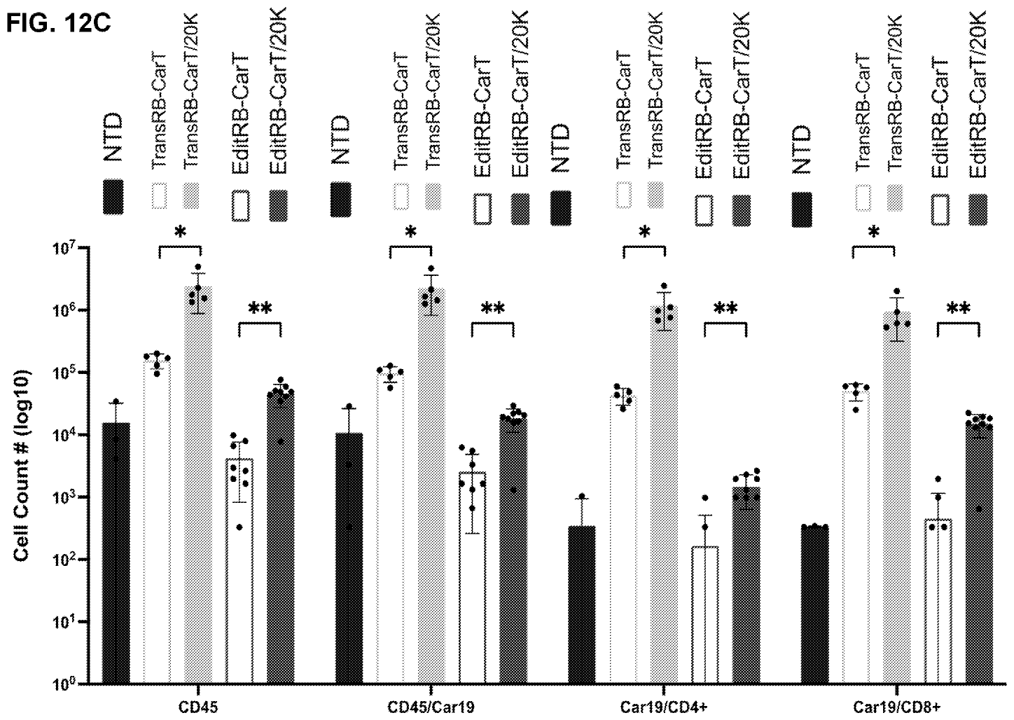

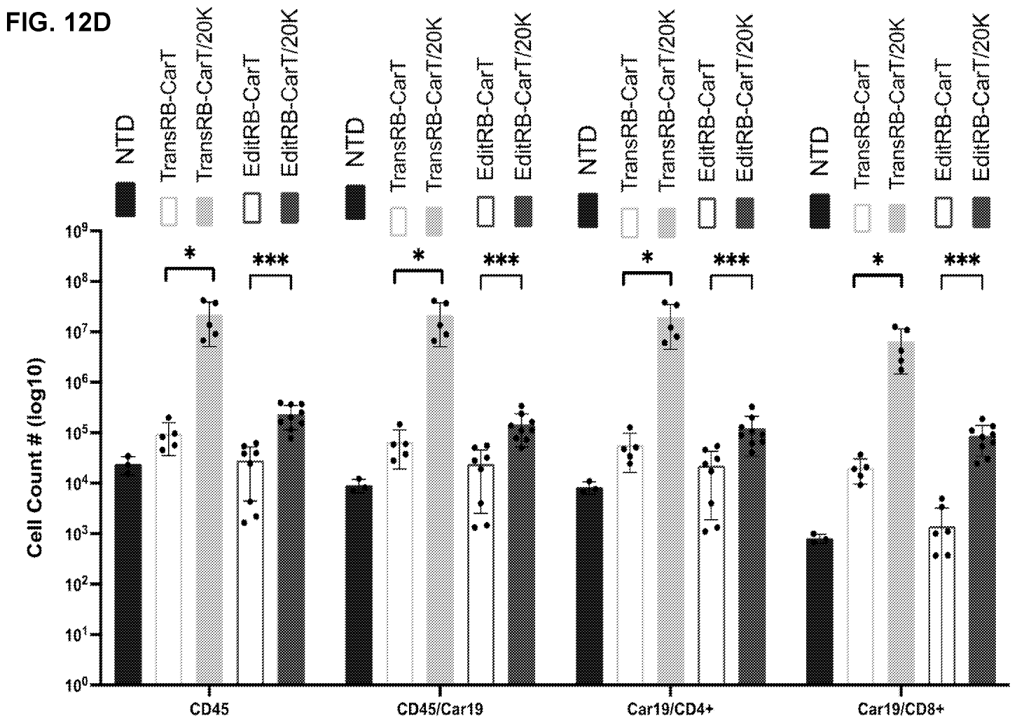

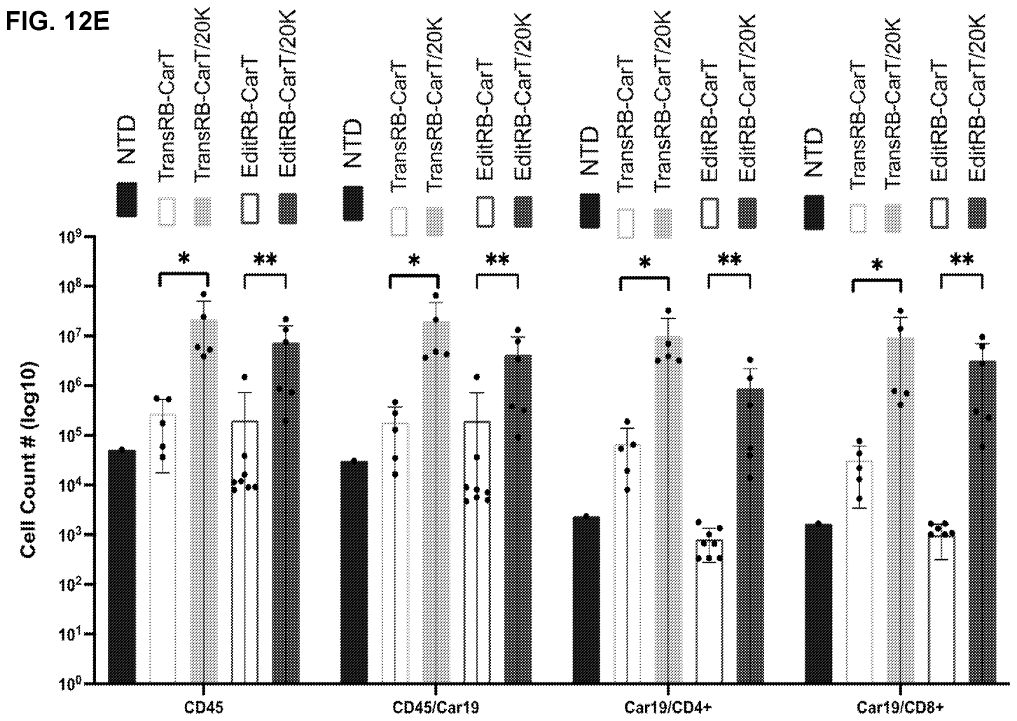

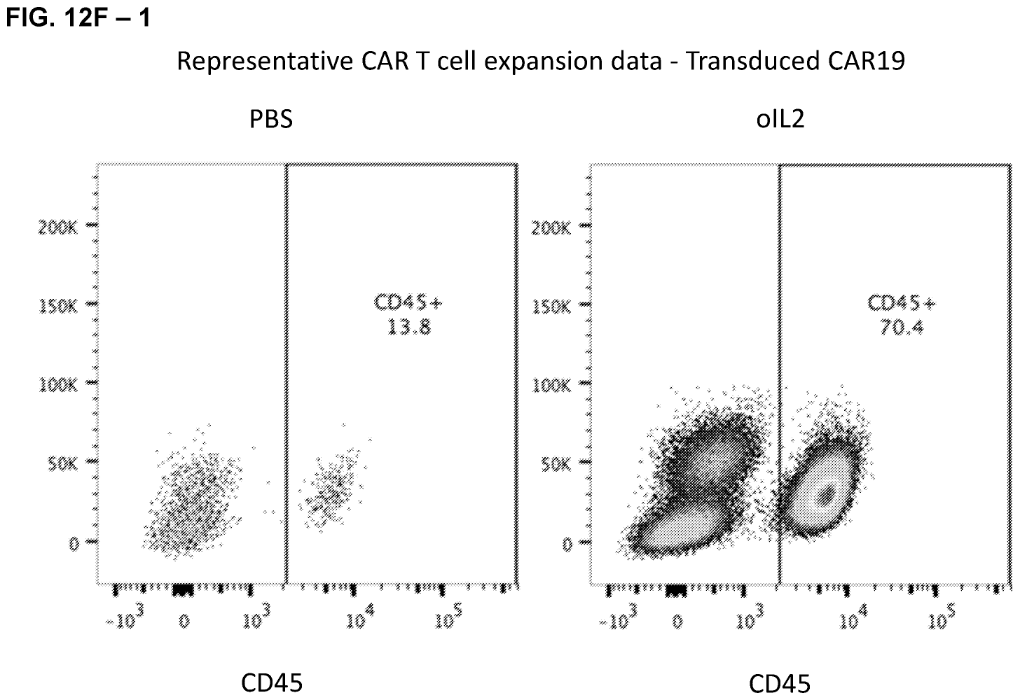

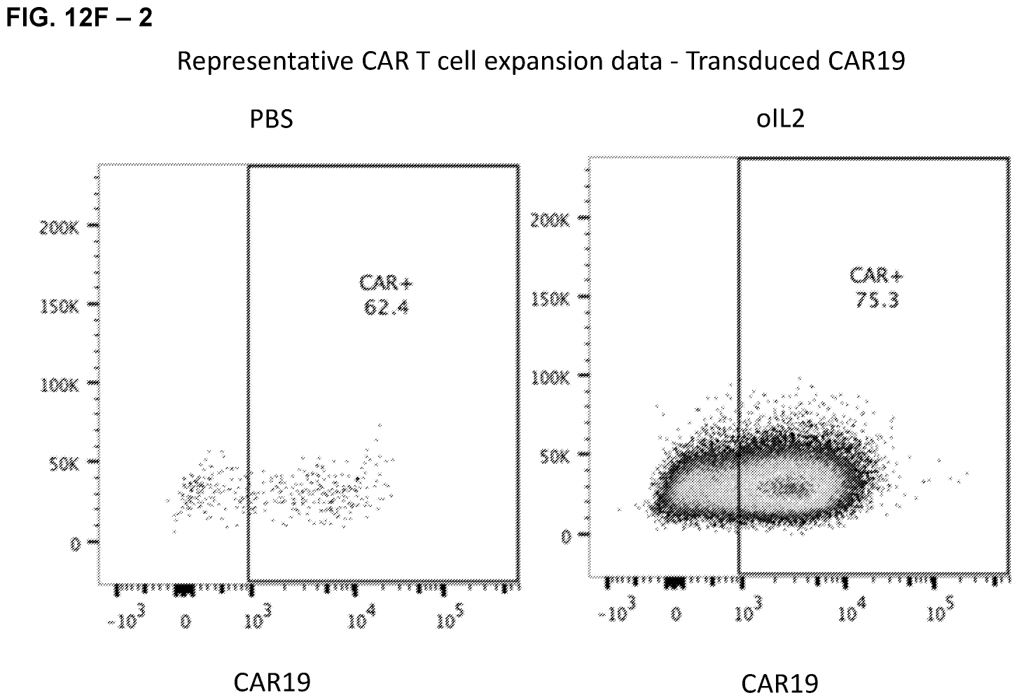

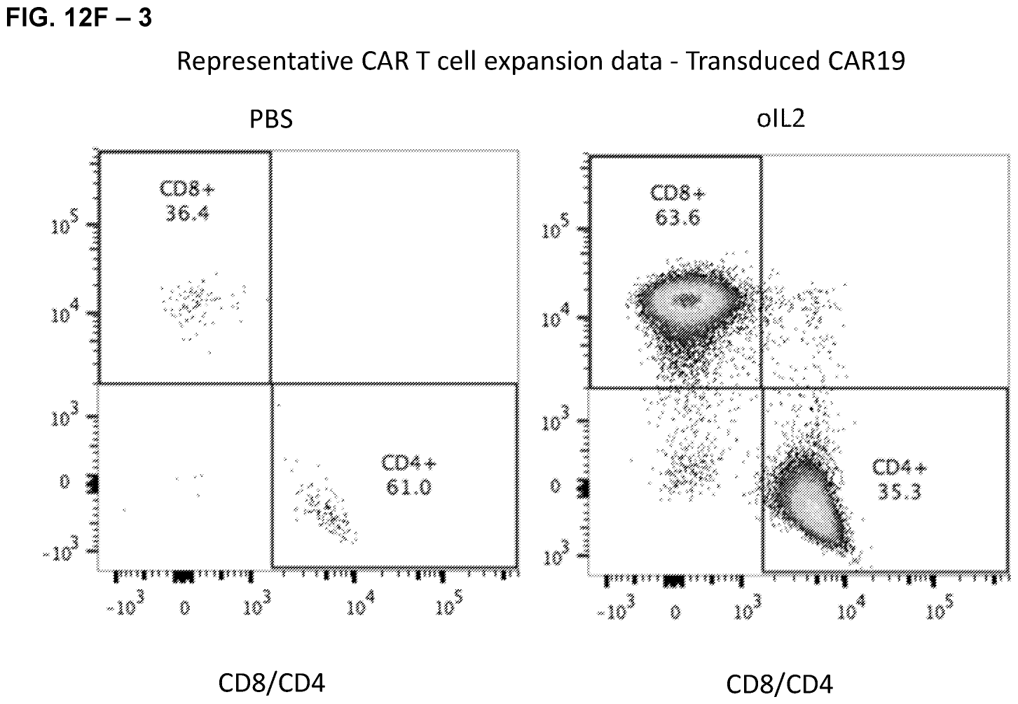

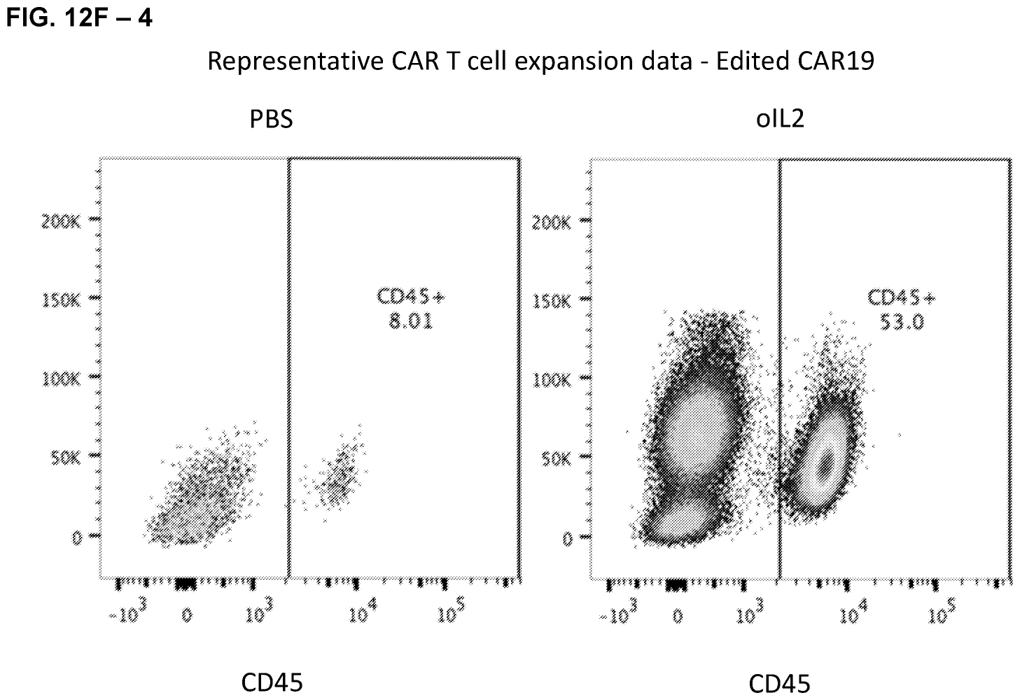

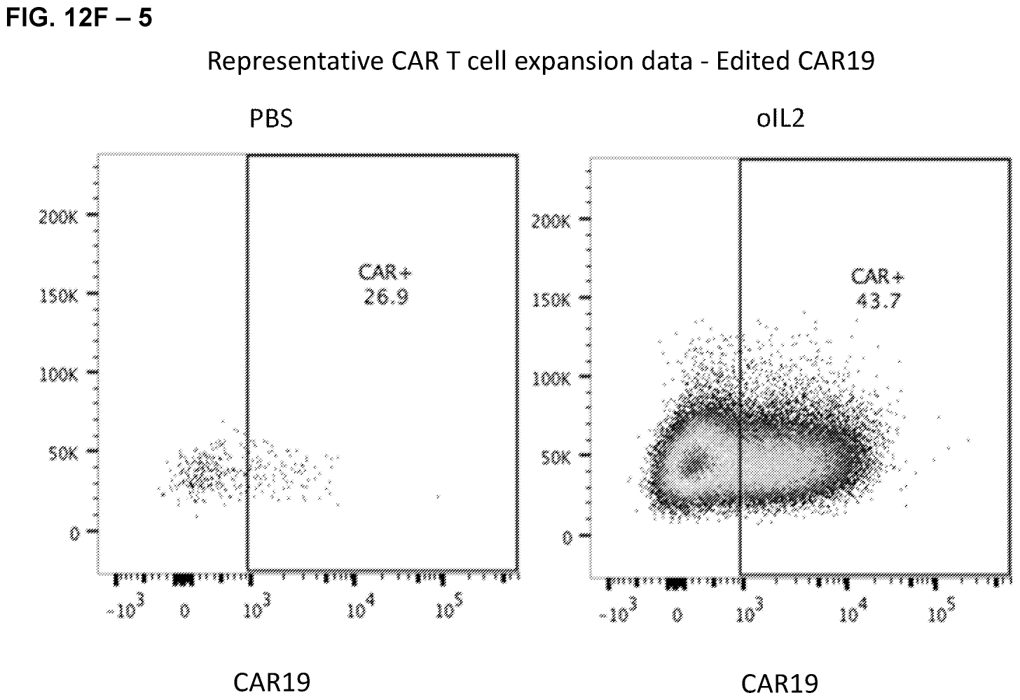

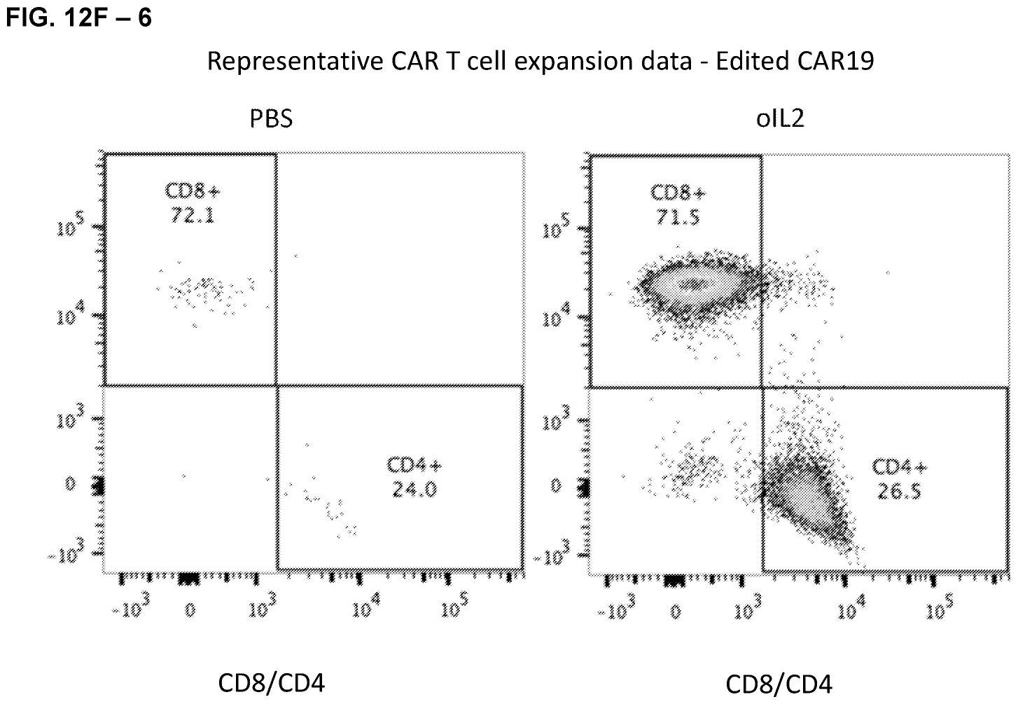

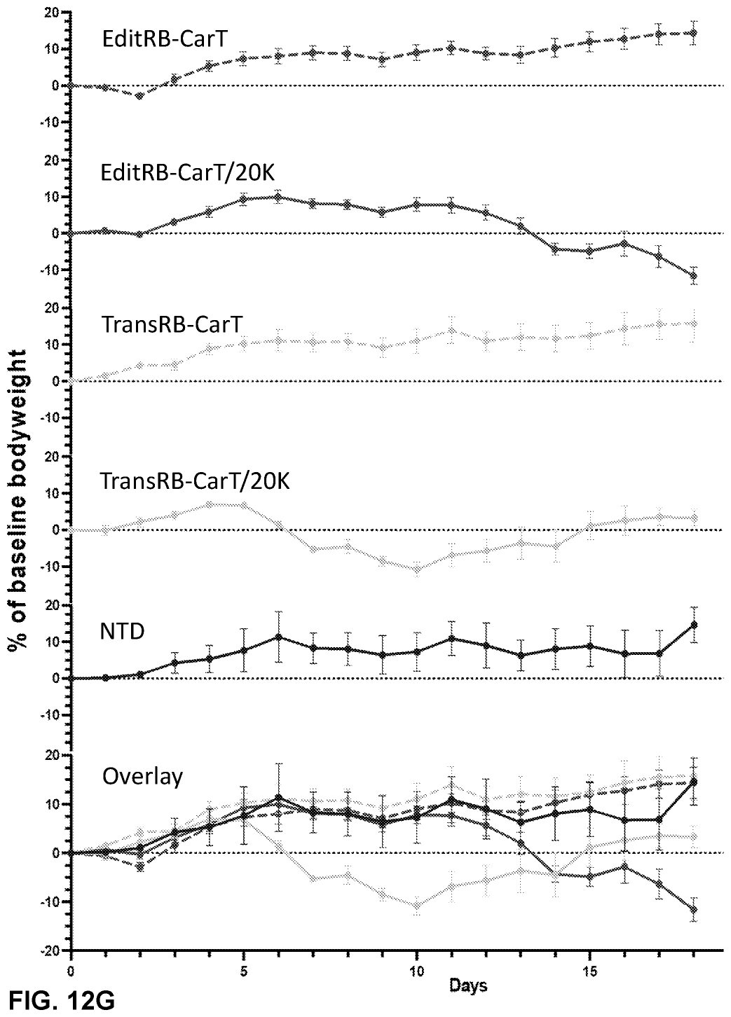

In a similar in vivo experiment, the engineered orthoIL2Rb+IL2−/− CAR T cell described herein (“edited” cells) were compared in vivo with T cells transduced with a lentiviral vector to express both orthoIL2Rb and CAR19 (“transduced” cells) (FIG. 12A). BLI intensity of Nalm6-LUC was determined for mice infused with the various T cells (edited or transduced) and receiving PBS or oIL2 (FIG. 12B). CAR T cell expansion was measured at weeks 1, 2, and 3 (FIGS. 12C-12E). Representative FACS flow plots of the CAR T cell expansion are shown in FIG. 12F. Mouse body weight over time was normalized to the body weight on day 0 for each mouse receiving PBS or 20K IU of oIL2 (FIG. 12G). Together, the data indicate that the engineered orthoCAR19 T cells described herein show anti-leukemic activity.

### Enumerated Embodiments

The following enumerated embodiments are provided, the numbering of which is not to be construed as designating levels of importance.

Embodiment 1: A method of producing a modified immune cell responsive to orthogonal cytokine signaling, the method comprising:
(a) genetically engineering an immune effector cell responsive to interleukin-2 (IL-2) and interleukin-15 (IL-15) to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell; and
(b) genetically engineering the immune effector cell to express an orthogonal IL-2 receptor beta (oIL2Rβ);
wherein step (a) and step (b) are performed in any order.
   Embodiment 2: The method of embodiment 1, wherein step (b) comprises genetically engineering endogenous IL-2 receptor beta (IL2Rβ) gene of the immune effector cell to express the oIL2Rβ such that the modified immune cell is an endogenous IL2Rβ−/− immune cell and an oIL2Rβ+/+ immune cell.
   Embodiment 3: The method of embodiment 1 or embodiment 2, wherein step (a) comprises a clustered regularly interspaced short palindromic repeats (CRISPR) associated nuclease (Cas nuclease) and a single-guide RNA (sgRNA) that targets the Cas nuclease to the locus within the endogenous IL-2 gene of the immune cell.
   Embodiment 4: The method of embodiment 3, wherein the Cas nuclease is a Cas9 nuclease.
   Embodiment 5: The method of embodiment 3 or embodiment 4, wherein step (a) comprises CRISPR/Cas-mediated homology directed repair (HDR).
   Embodiment 6: The method of any one of the preceding embodiments, wherein the genetic engineering of step (b) comprises prime editing.
   Embodiment 7: The method of embodiment 6, wherein the prime editing comprises a Cas9 nickase-reverse transcriptase and a prime editing guide RNA (pegRNA).
   Embodiment 8: The method of embodiment 6 or embodiment 7, wherein the immune cell is a human immune cell, further wherein the prime editing comprises introducing a first point mutation and a second point mutation into the endogenous IL2Rβ gene, wherein the first point mutation results in a H133D amino acid change and and the second point mutation results in a Y134F amino acid change.
   Embodiment 9: The method of embodiment 8, wherein the first point mutation is C397G and the second point mutation is A401T.
   Embodiment 10: The method of any one of the preceding embodiments, wherein the immune cell is a human immune cell, further wherein the oIL2Rβ comprises H133D and Y134F mutations relative to endogenous IL2Rβ.
   Embodiment 11: The method of any one of the preceding embodiments, wherein the modified immune cell is responsive to an orthogonal IL-2 (oIL2).
   Embodiment 12: The method of embodiment 11, wherein the oIL2 binds to the oIL2Rβ.
   Embodiment 13: The method of any one of the preceding embodiments, wherein the immune cell is a T cell.
   Embodiment 14: The method of any one of the preceding embodiments, wherein the immune cell is a human T cell.
   Embodiment 15: The method of any one of the preceding embodiments, wherein:
step (a) comprises genetically engineering the immune cell to express a TCR, and wherein the TCR targets a tumor antigen; or
step (a) comprises genetically engineering the immune cell to express a CAR, and wherein the CAR targets a tumor antigen.
   Embodiment 16: The method of embodiment 15, wherein the tumor antigen is selected from the group consisting of CD19, CD20, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, gp100, Glycolipid F77, FAP, EGFRvIII, MAGE A3, 5T4, WT1, KG2D ligand, folate receptor alpha (FRa), and a Wnt1 antigen.
   Embodiment 17: The method of any one of the preceding embodiments, wherein the CAR comprises an extracellular antigen binding domain, a transmembrane domain, and an intracellular domain.
   Embodiment 18: The method of embodiment 17, wherein the antigen binding domain is selected from the group consisting of a full-length antibody or antigen-binding fragment thereof, a Fab, a single-chain variable fragment (scFv), or a single-domain antibody.
   Embodiment 19: The method of embodiment 18, wherein the antigen binding domain is an scFv.
   Embodiment 20: The method of embodiment 19, wherein the antigen binding domain is an anti-CD19 scFv.
   Embodiment 21: The method of any one of embodiments 17-20, wherein the intracellular domain of the CAR comprises:
a costimulatory domain, or a variant thereof, of a protein selected from the group consisting of a protein in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, B7-H3 (CD276), and any combination thereof; or
an intracellular domain derived from a killer immunoglobulin-like receptor (KIR).
   Embodiment 22: The method of any one of embodiments 17-21, wherein the intracellular domain of the CAR comprises or further comprises an intracellular signaling domain, or a variant thereof, of a protein selected from the group consisting of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d.
   Embodiment 23: The method of any one of the preceding embodiments, wherein the CAR comprises an anti-CD19 scFv, a transmembrane domain, and an intracellular domain comprising a 4-1BB costimulatory domain and a CD3 zeta signaling domain.
   Embodiment 24: A modified immune cell responsive to orthogonal cytokine signaling, wherein the modified immune cell is derived from an immune effector cell responsive to interleukin-2 (IL-2) and interleukin-15 (IL-15); and wherein the modified immune cell:
(a) expresses a T cell receptor (TCR) or a chimeric antigen receptor (CAR) from an exogenous nucleic acid inserted at a locus within endogenous IL-2 gene of the immune cell, wherein the exogenous nucleic acid comprises a polynucleotide sequence encoding the TCR or the CAR, such that the modified immune cell is an IL2−/− immune cell; and
(b) expresses an orthogonal IL-2 receptor beta (oIL2Rβ).
   Embodiment 25: The modified immune cell of embodiment 24, wherein the modified immune cell is an endogenous IL2Rβ−/− immune cell.
   Embodiment 26: The modified immune cell of embodiment 24 or embodiment 25, wherein the endogenous IL2Rβ gene is edited such that it encodes the oIL2Rβ.
   Embodiment 27: The modified immune cell of embodiment 26, wherein the immune effector cell is a human immune cell, further wherein the edited endogenous IL2Rβ gene comprises a first point mutation and a second point mutation, wherein the first point mutation results in a H133D amino acid change and and the second point mutation results in a Y134F amino acid change relative to endogenous IL2Rβ.
   Embodiment 28: The modified immune cell of embodiment 27, wherein the first point mutation is C397G and the second point mutation is A401T.
   Embodiment 29: The modified immune cell of any one of the preceding embodiments, wherein the immune effector cell is a human immune cell, further wherein the oIL2Rβ comprises H133D and Y134F mutations relative to IL2Rβ.
   Embodiment 30: The modified immune cell of any one of the preceding embodiments, wherein the modified immune cell is responsive to an orthogonal IL-2 (oIL2).
   Embodiment 31: The modified immune cell of embodiment 30, wherein the oIL2 binds to the oIL2Rβ.
   Embodiment 32: The modified immune cell of any one of the preceding embodiments, wherein the immune effector cell is a T cell.
   Embodiment 33: The modified immune cell of any one of the preceding embodiments, wherein the immune effector cell is a human T cell.
   Embodiment 34: The modified immune cell of any one of the preceding embodiments, wherein the TCR targets a tumor antigen, or wherein the CAR targets a tumor antigen.
   Embodiment 35: The modified immune cell of embodiment 33, wherein the tumor antigen is selected from the group consisting of CD19, CD20, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, gp100, Glycolipid F77, FAP, EGFRvIII, MAGE A3, 5T4, WT1, KG2D ligand, folate receptor alpha (FRa), and a Wnt1 antigen.
   Embodiment 36: The modified immune cell of any one of the preceding embodiments, wherein the CAR comprises an extracellular antigen binding domain, a transmembrane domain, and an intracellular domain.
   Embodiment 37: The modified immune cell of embodiment 36, wherein the antigen binding domain is selected from the group consisting of a full-length antibody or antigen-binding fragment thereof, a Fab, a single-chain variable fragment (scFv), or a single-domain antibody.
   Embodiment 38: The modified immune cell embodiment 37, wherein the antigen binding domain is an scFv.
   Embodiment 39: The modified immune cell of embodiment 38, wherein the antigen binding domain is an anti-CD19 scFv.
   Embodiment 40: The modified immune cell of any one of embodiments 36-39, wherein the intracellular domain of the CAR comprises:
a costimulatory domain, or a functional variant thereof, of a protein selected from the group consisting of a protein in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, B7-H3 (CD276), and any combination thereof; or
an intracellular domain derived from a killer immunoglobulin-like receptor (KIR).
   Embodiment 41: The modified immune cell of any one of embodiments 36-40, wherein the intracellular domain of the CAR comprises or further comprises an intracellular signaling domain, or a functional variant thereof, of a protein selected from the group consisting of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d.
   Embodiment 42: The modified immune cell of any one of the preceding embodiments, wherein the CAR comprises an anti-CD19 scFv, a transmembrane domain, and an intracellular domain comprising a 4-1BB costimulatory domain and a CD3 zeta signaling domain.
   Embodiment 43: A modified immune cell responsive to orthogonal cytokine signaling, wherein the modified immune cell is produced by the method of any one of embodiments 1-23.
   Embodiment 44: A method of treating cancer in a subject, the method comprising:
(a) administering to the subject an effective amount of the modified immune cell responsive to orthogonal cytokine signaling of any one of embodiments 23-41; and
(b) administering to the subject an effective amount of an orthogonal interleukin-2 (oIL2) which binds to the oIL2Rβ, or a vector which expresses the oIL2.
   Embodiment 45: The method of embodiment 44, wherein the vector which expresses oIL2 is a viral vector.
   Embodiment 46: The method of embodiment 45, wherein the viral vector is selected from an adenoviral vector, an adeno-associated virus (AAV) vector, a lentiviral vector, and a retroviral vector.
   Embodiment 47: The method of any one of embodiments 44-46, wherein administering comprises intravenous administration and/or intratumoral injection.
   Embodiment 48: The method of any one of embodiments 44-47, wherein the immune effector cell is a human cell and wherein the subject is a human.
   Embodiment 49: The method of any one of embodiments 44-48, wherein the immune effector cell is a human T cell and wherein the subject is a human.
   Embodiment 50: The method of any one of embodiments 44-49, wherein the method further comprises discontinuing administration of the oIL2 or the vector which expresses the oIL2.
   Embodiment 51: A method of producing a modified immune cell responsive to orthogonal cytokine signaling, the method comprising:
(a) genetically engineering an immune effector cell responsive to interleukin-2 (IL-2) and interleukin-15 (IL-15) to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell; and
(b) genetically engineering endogenous IL-2 receptor beta (IL2Rβ) gene of the immune effector cell to express the oIL2Rβ such that the modified immune cell is an endogenous IL2Rβ−/− immune cell and an oIL2Rβ+/+ immune cell;
wherein step (a) and step (b) are performed in any order; and
further wherein step (a) comprises CRISPR/Cas-mediated homology directed repair (HDR) and step (b) comprises prime editing.
   Embodiment 52: A modified immune cell responsive to orthogonal cytokine signaling, wherein the modified immune cell is derived from an immune effector cell responsive to interleukin-2 (IL-2) and interleukin-15 (IL-15); and wherein the modified immune cell:
(a) expresses a T cell receptor (TCR) or a chimeric antigen receptor (CAR) from an exogenous nucleic acid inserted at a locus within endogenous IL-2 gene of the immune cell, wherein the exogenous nucleic acid comprises a polynucleotide sequence encoding the TCR or the CAR, such that the modified immune cell is an IL2−/− immune cell; and
(b) expresses an orthogonal IL-2 receptor beta (oIL2Rβ); wherein the endogenous IL2Rβ gene is edited such that it encodes the oIL2Rβ.
   Embodiment 53: A method of producing a modified immune cell responsive to orthogonal cytokine signaling, the method comprising genetically engineering at least one endogenous IL-2 receptor beta (IL2Rβ) gene of the immune effector cell to express an orthogonal IL-2 receptor beta (oIL2Rβ), wherein the modified immune cell is derived from an immune effector cell responsive to interleukin-2 (IL-2) and interleukin-15 (IL-15); further wherein the genetic engineering comprises prime editing, and wherein the prime editing comprises a prime editing guide RNA (pegRNA) comprising or consisting of SEQ ID NO: 1.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of producing a modified immune cell responsive to orthogonal cytokine signaling, the method comprising:
(a) genetically engineering an immune effector cell responsive to interleukin-2 (IL-2) and interleukin-15 (IL-15) to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell; and
(b) genetically engineering the immune effector cell to express an orthogonal IL-2 receptor beta (oIL2Rβ);
wherein step (a) and step (b) are performed in any order.

2. The method of claim 1, wherein step (b) comprises genetically engineering endogenous IL-2 receptor beta (IL2Rβ) gene of the immune effector cell to express the oIL2Rβ such that the modified immune cell is an endogenous IL2Rβ−/− immune cell and an oIL2Rβ+/+ immune cell.

3. The method of claim 1, wherein step (a) comprises a clustered regularly interspaced short palindromic repeats (CRISPR) associated nuclease (Cas nuclease) and a single-guide RNA (sgRNA) that targets the Cas nuclease to the locus within the endogenous IL-2 gene of the immune cell.

4. The method of claim 3, wherein the Cas nuclease is a Cas9 nuclease.

5. The method of claim 3, wherein step (a) comprises CRISPR/Cas-mediated homology directed repair (HDR).

6. The method of claim 1, wherein the genetic engineering of step (b) comprises prime editing.

7. The method of claim 6, wherein the prime editing comprises a Cas9 nickase-reverse transcriptase and a prime editing guide RNA (pegRNA).

8. The method of claim 6, wherein the immune cell is a human immune cell, further wherein the prime editing comprises introducing a first point mutation and a second point mutation into the endogenous IL2Rβ gene, wherein the first point mutation results in a H133D amino acid change and and the second point mutation results in a Y134F amino acid change.

9. The method of claim 8, wherein the first point mutation is C397G and the second point mutation is A401T.

10. The method of claim 1, wherein the immune cell is a human immune cell, further wherein the oIL2Rβ comprises H133D and Y134F mutations relative to endogenous IL2Rβ.

11. The method of claim 1, wherein the modified immune cell is responsive to an orthogonal IL-2 (oIL2).

12. The method of claim 11, wherein the oIL2 binds to the oIL2Rβ.

13. The method of claim 1, wherein the immune cell is a T cell.

14. The method of claim 1, wherein the immune cell is a human T cell.

15. The method of claim 1, wherein:
step (a) comprises genetically engineering the immune cell to express a TCR, and wherein the TCR targets a tumor antigen; or
step (a) comprises genetically engineering the immune cell to express a CAR, and wherein the CAR targets a tumor antigen.

16. The method of claim 15, wherein the tumor antigen is selected from the group consisting of CD19, CD20, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, gp100, Glycolipid F77, FAP, EGFRvIII, MAGE A3, 5T4, WT1, KG2D ligand, folate receptor alpha (FRa), and a Wnt1 antigen.

17. The method of claim 1, wherein the CAR comprises an extracellular antigen binding domain, a transmembrane domain, and an intracellular domain.

18. The method of claim 17, wherein the antigen binding domain is selected from the group consisting of a full-length antibody or antigen-binding fragment thereof, a Fab, a single-chain variable fragment (scFv), or a single-domain antibody.

19. The method of claim 18, wherein the antigen binding domain is an scFv.

20. The method of claim 19, wherein the antigen binding domain is an anti-CD19 scFv.

21. The method of claim 17, wherein the intracellular domain of the CAR comprises:
a costimulatory domain, or a variant thereof, of a protein selected from the group consisting of a protein in the TNFR superfamily, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS, NKG2C, B7-H3 (CD276), and any combination thereof; or
an intracellular domain derived from a killer immunoglobulin-like receptor (KIR).

22. The method of claim 17, wherein the intracellular domain of the CAR comprises or further comprises an intracellular signaling domain, or a variant thereof, of a protein selected from the group consisting of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d.

23. The method of claim 1, wherein the CAR comprises an anti-CD19 scFv, a transmembrane domain, and an intracellular domain comprising a 4-1BB costimulatory domain and a CD3 zeta signaling domain.

24. A modified immune cell responsive to orthogonal cytokine signaling, wherein the modified immune cell is derived from an immune effector cell responsive to interleukin-2 (IL-2) and interleukin-15 (IL-15); and wherein the modified immune cell:
(a) expresses a T cell receptor (TCR) or a chimeric antigen receptor (CAR) from an exogenous nucleic acid inserted at a locus within endogenous IL-2 gene of the immune cell, wherein the exogenous nucleic acid comprises a polynucleotide sequence encoding the TCR or the CAR, such that the modified immune cell is an IL2−/− immune cell; and
(b) expresses an orthogonal IL-2 receptor beta (oIL2Rβ).

25. The modified immune cell of claim 24, wherein the modified immune cell is an endogenous IL2Rβ−/− immune cell.

26. The modified immune cell of claim 24, wherein the endogenous IL2Rβ gene is edited such that it encodes the oIL2Rβ.

27. The modified immune cell of claim 26, wherein the immune effector cell is a human immune cell, further wherein the edited endogenous IL2Rβ gene comprises a first point mutation and a second point mutation, wherein the first point mutation results in a H133D amino acid change and and the second point mutation results in a Y134F amino acid change relative to endogenous IL2Rβ.

28. The modified immune cell of claim 27, wherein the first point mutation is C397G and the second point mutation is A401T.

29. The modified immune cell of claim 24, wherein the immune effector cell is a human immune cell, further wherein the oIL2Rβ comprises H133D and Y134F mutations relative to IL2Rβ.

30. The modified immune cell of claim 24, wherein the modified immune cell is responsive to an orthogonal IL-2 (oIL2).

31. The modified immune cell of claim 30, wherein the oIL2 binds to the oIL2Rβ.

32. The modified immune cell of claim 24, wherein the immune effector cell is a T cell.

33. The modified immune cell of claim 24, wherein the immune effector cell is a human T cell.

34. The modified immune cell of claim 24, wherein the TCR targets a tumor antigen, or wherein the CAR targets a tumor antigen.

35. The modified immune cell of claim 34, wherein the tumor antigen is selected from the group consisting of CD19, CD20, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, gp100, Glycolipid F77, FAP, EGFRvIII, MAGE A3, 5T4, WT1, KG2D ligand, folate receptor alpha (FRa), and a Wnt1 antigen.

36. The modified immune cell of claim 24, wherein the CAR comprises an extracellular antigen binding domain, a transmembrane domain, and an intracellular domain.

37. The modified immune cell of claim 36, wherein the antigen binding domain is selected from the group consisting of a full-length antibody or antigen-binding fragment thereof, a Fab, a single-chain variable fragment (scFv), or a single-domain antibody.

38. The modified immune cell of claim 37, wherein the antigen binding domain is an scFv.

39. The modified immune cell of claim 38, wherein the antigen binding domain is an anti-CD19 scFv.

40. The modified immune cell of claim 36, wherein the intracellular domain of the CAR comprises:
a costimulatory domain, or a functional variant thereof, of a protein selected from the group consisting of a protein in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, B7-H3 (CD276), and any combination thereof; or
an intracellular domain derived from a killer immunoglobulin-like receptor (KIR).

41. The modified immune cell of claim 36, wherein the intracellular domain of the CAR comprises or further comprises an intracellular signaling domain, or a functional variant thereof, of a protein selected from the group consisting of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d.

42. The modified immune cell of claim 24, wherein the CAR comprises an anti-CD19 scFv, a transmembrane domain, and an intracellular domain comprising a 4-1BB costimulatory domain and a CD3 zeta signaling domain.

43. A modified immune cell responsive to orthogonal cytokine signaling, wherein the modified immune cell is produced by the method of claim 1.

44. A method of treating cancer in a subject, the method comprising:
(a) administering to the subject an effective amount of the modified immune cell responsive to orthogonal cytokine signaling of claim 24; and
(b) administering to the subject an effective amount of an orthogonal interleukin-2 (oIL2) which binds to the oIL2Rβ, or a vector which expresses the oIL2.

45. The method of claim 44, wherein the vector which expresses oIL2 is a viral vector.

46. The method of claim 45, wherein the viral vector is selected from an adenoviral vector, an adeno-associated virus (AAV) vector, a lentiviral vector, and a retroviral vector.

47. The method of claim 44, wherein administering comprises intravenous administration and/or intratumoral injection.

48. The method of claim 44, wherein the immune effector cell is a human cell and wherein the subject is a human.

49. The method of claim 44, wherein the immune effector cell is a human T cell and wherein the subject is a human.

50. The method of claim 44, wherein the method further comprises discontinuing administration of the oIL2 or the vector which expresses the oIL2.

51. A method of producing a modified immune cell responsive to orthogonal cytokine signaling, the method comprising:
(a) genetically engineering an immune effector cell responsive to interleukin-2 (IL-2) and interleukin-15 (IL-15) to express a T cell receptor (TCR) or a chimeric antigen receptor (CAR) from an exogenous nucleic acid inserted at a locus within endogenous IL-2 gene of the immune cell such that the modified immune cell is an IL-2−/− immune cell; and
(b) genetically engineering endogenous IL-2 receptor beta (IL2Rβ) gene of the immune effector cell to express an orthogonal IL-2 receptor beta (oIL2Rβ) such that the modified immune cell is an endogenous IL2Rβ−/− immune cell and an oIL2Rβ+/+ immune cell;
wherein step (a) and step (b) are performed in any order; and
further wherein step (a) comprises CRISPR/Cas-mediated homology directed repair (HDR) and step (b) comprises prime editing.

52. A modified immune cell responsive to orthogonal cytokine signaling, wherein the modified immune cell is derived from an immune effector cell responsive to interleukin-2 (IL-2) and interleukin-15 (IL-15); and wherein the modified immune cell:
(a) expresses a T cell receptor (TCR) or a chimeric antigen receptor (CAR) from an exogenous nucleic acid inserted at a locus within endogenous IL-2 gene of the immune cell, wherein the exogenous nucleic acid comprises a polynucleotide sequence encoding the TCR or the CAR, such that the modified immune cell is an IL2−/− immune cell; and
(b) expresses an orthogonal IL-2 receptor beta (oIL2Rβ);
wherein the endogenous IL2Rβ gene is edited such that it encodes the oIL2Rβ.

53. A method of producing a modified immune cell responsive to orthogonal cytokine signaling, the method comprising genetically engineering at least one endogenous IL-2 receptor beta (IL2Rβ) gene of the immune effector cell to express an orthogonal IL-2 receptor beta (oIL2Rβ), wherein the modified immune cell is derived from an immune effector cell responsive to interleukin-2 (IL-2) and interleukin-15 (IL-15); further wherein the genetic engineering comprises prime editing, and wherein the prime editing comprises a prime editing guide RNA (pegRNA) comprising or consisting of SEQ ID NO: 1.
